SEQUENCE LISTING (104 sequences)

SEQ ID NO: 1 - protein, 209 aa, Homo sapiens
MDSDETGFEHSGLWVSVLAGLLLGACQAHPIPDSSPLLQPGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 2 - protein, 210 aa, Artificial Sequence
  FGF-21 mutein G + FGF-21 (including signal sequence)
GMDSDETGFEHSGLWVSVLAGLLLGACQAHPIPDSSPLLQPGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 3 - protein, 181 aa, Artificial Sequence
  FGF-21 mutein H29-S209
HPIPDSSPLLQPGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 4 - protein, 39 aa, Artificial Sequence
  Exenatide
  MOD_RES (39)..(39): AMIDATION
HGEGTFTSDLSKQMEEEAVRLFIEWLKNGGPSSGAPPPS

SEQ ID NO: 5 - protein, 31 aa, Artificial Sequence
  Human GLP-1(7-37)
HAEGTFTSDVSSYLEGQAAKEFIAWLVKGRG

SEQ ID NO: 6 - protein, 37 aa, Artificial Sequence
  Oxyntomodulin
HSQGTFTSDYSKYLDSRRAQDFVQWLMNTKRNRNNIA

SEQ ID NO: 7 - protein, 30 aa, Artificial Sequence
  Human GLP-1(7-36)NH2
  MOD_RES (30)..(30): AMIDATION
HAEGTFTSDVSSYLEGQAAKEFIAWLVKGR

SEQ ID NO: 8 - protein, 39 aa, Artificial Sequence; identical to SEQ ID NO: 4
  Exendin-4
  MOD_RES (39)..(39): AMIDATION

SEQ ID NO: 9 - protein, 44 aa, Artificial Sequence
  Lixisenatide
  MOD_RES (44)..(44): AMIDATION
HGEGTFTSDLSKQMEEEAVRLFIEWLKNGGPSSGAPPSKKKKKK

SEQ ID NO: 10 - protein, 44 aa, Artificial Sequence; identical to SEQ ID NO: 9
  Lixisenatide
  MOD_RES (44)..(44): AMIDATION

SEQ ID NO: 11 - protein, 4 aa, Artificial Sequence
  Factor Xa cleavage site
IEGR

SEQ ID NO: 12 - protein, 7 aa, Artificial Sequence
  Pasylation unit sequence
APASPAS

SEQ ID NO: 13 - protein, 8 aa, Artificial Sequence
  Pasylation sequence with site for covalent modification (C)
APASCPAS

SEQ ID NO: 14 - protein, 5 aa, Artificial Sequence
  Protease cleavage site
GGGRR

SEQ ID NO: 15 - protein, 224 aa, Artificial Sequence
  Exenatide-FactorXa-cleavage site-FGF21
HGEGTFTSDLSKQMEEEAVRLFIEWLKNGGPSSGAPPPSIEGRHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 16 - protein, 332 aa, Artificial Sequence
  His-SUMO-Exenatide-FactorXa-cleavage site-FGF21
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPPSIEGRHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 17 - protein, 220 aa, Artificial Sequence
  Exenatide-FGF21
HGEGTFTSDLSKQMEEEAVRLFIEWLKNGGPSSGAPPPSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 18 - protein, 328 aa, Artificial Sequence
  His-SUMO-Exenatide-FGF21
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPPSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 19 - protein, 333 aa, Artificial Sequence
  His-SUMO-Exenatide-GGGRR-FGF21
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPPSGGGRRHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 20 - protein, 225 aa, Artificial Sequence
  Exenatide-GGGRR-FGF21
HGEGTFTSDLSKQMEEEAVRLFIEWLKNGGPSSGAPPPSGGGRRHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 21 - protein, 333 aa, Artificial Sequence
  His-SUMO-Lixisenatide-FGF21
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPSKKKKKKHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 22 - protein, 225 aa, Artificial Sequence
  Lixisenatide-FGF21
HGEGTFTSDLSKQMEEEAVRLFIEWLKNGGPSSGAPPSKKKKKKHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 23 - protein, 337 aa, Artificial Sequence
  His-SUMO-Lixisenatide-FactorXa-cleavage site-FGF21
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPSKKKKKKIEGRHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 24 - protein, 229 aa, Artificial Sequence
  Lixisenatide-FactorXa-cleavage site-FGF21
HGEGTFTSDLSKQMEEEAVRLFIEWLKNGGPSSGAPPSKKKKKKIEGRHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 25 - protein, 338 aa, Artificial Sequence
  His-SUMO-Lixisenatide-GGGRR-FGF21
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPSKKKKKKGGGRRHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 26 - protein, 230 aa, Artificial Sequence
  Lixisenatide-GGGRR-FGF21
HGEGTFTSDLSKQMEEEAVRLFIEWLKNGGPSSGAPPSKKKKKKGGGRRHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 27 - DNA, 999 bp, Artificial Sequence
  CR8829
atgggccatcaccatcaccatcacggaagcctgcaggatagcgaagttaatcaggaagcaaaaccggaagttaaaccggaagttaaaccggaaacccatattaatctgaaagttagcgatggtagcagcgaaattttttttaaaattaaaaaaaccaccccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcacatcgtgaacagattggtggtcatggtgaaggtacattcacatctgatctatcaaaacaaatggaagaagaagctgttagactattcattgaatggttgaaaaatggtggtccatcttcaggtgctccacctccaagtatcgaaggtcgtcaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcctga

SEQ ID NO: 28 - DNA, 987 bp, Artificial Sequence
  CR8846
atgggccatcaccatcaccatcacggaagcctgcaggatagcgaagttaatcaggaagcaaaaccggaagttaaaccggaagttaaaccggaaacccatattaatctgaaagttagcgatggtagcagcgaaattttttttaaaattaaaaaaaccaccccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcacatcgtgaacagattggtggtcatggtgaaggtacattcacatctgatctatcaaaacaaatggaagaagaagctgttagactattcattgaatggttgaaaaatggtggtccatcttcaggtgctccacctccaagtcaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcctga

SEQ ID NO: 29 - DNA, 1002 bp, Artificial Sequence
  CR8847
atgggccatcaccatcaccatcacggaagcctgcaggatagcgaagttaatcaggaagcaaaaccggaagttaaaccggaagttaaaccggaaacccatattaatctgaaagttagcgatggtagcagcgaaattttttttaaaattaaaaaaaccaccccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcacatcgtgaacagattggtggtcatggtgaaggtacattcacatctgatctatcaaaacaaatggaagaagaagctgttagactattcattgaatggttgaaaaatggtggtccatcttcaggtgctccacctccaagtgggggcgggcgccgacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcctga

SEQ ID NO: 30 - DNA, 1002 bp, Artificial Sequence
  CR8848
atgggccatcaccatcaccatcacggaagcctgcaggatagcgaagttaatcaggaagcaaaaccggaagttaaaccggaagttaaaccggaaacccatattaatctgaaagttagcgatggtagcagcgaaattttttttaaaattaaaaaaaccaccccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcacatcgtgaacagattggtggtcacggtgaaggtaccttcacctccgacctgtccaaacagatggaagaagaagctgttcgtctgttcatcgaatggctgaaaaacggtggtccgtcctccggtgctccgccttcgaaaaagaagaaaaagaaacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcctga

SEQ ID NO: 31 - DNA, 1014 bp, Artificial Sequence
  CR8849
atgggccatcaccatcaccatcacggaagcctgcaggatagcgaagttaatcaggaagcaaaaccggaagttaaaccggaagttaaaccggaaacccatattaatctgaaagttagcgatggtagcagcgaaattttttttaaaattaaaaaaaccaccccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcacatcgtgaacagattggtggtcacggtgaaggtaccttcacctccgacctgtccaaacagatggaagaagaagctgttcgtctgttcatcgaatggctgaaaaacggtggtccgtcctccggtgctccgccttcgaaaaagaagaaaaagaaaatcgaaggtcgtcaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcctga

SEQ ID NO: 32 - DNA, 1017 bp, Artificial Sequence
  CR8850
atgggccatcaccatcaccatcacggaagcctgcaggatagcgaagttaatcaggaagcaaaaccggaagttaaaccggaagttaaaccggaaacccatattaatctgaaagttagcgatggtagcagcgaaattttttttaaaattaaaaaaaccaccccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcacatcgtgaacagattggtggtcacggtgaaggtaccttcacctccgacctgtccaaacagatggaagaagaagctgttcgtctgttcatcgaatggctgaaaaacggtggtccgtcctccggtgctccgccttcgaaaaagaagaaaaagaaagggggcgggagaaggcaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcctga

SEQ ID NO: 33 - DNA, 1011 bp, Artificial Sequence
  CR9443
atgggacaccaccatcatcatcatggtagcctgcaggatagcgaagttaatcaagaagcaaaaccggaagttaaaccggaagtgaaaccggaaacccatattaatctgaaagttagtgatggcagcagcgaaattttctttaaaatcaaaaaaaccacaccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcccatcgtgaacaaattggtggtcatccgattccggatagcagtccgctgctgcagtttggtggtcaggttcgtcagcgttatctgtataccgatgatgcacagcagaccgaagcacatctggaaattcgtgaagatggcaccgttggtggtgcagcagatcagagtccggaaagcctgctgcagctgaaagcactgaaaccgggtgttattcagattctgggtgttaaaaccagccgttttctgtgtcagcgtccggatggtgcactgtatggtagtctgcattttgatccggaagcatgtagctttcgtgaactgctgctggaagatggttataatgtttatcagagtgaagcacatggtctgccgctgcatctgcctggtaataaaagtccgcatcgtgatccggcaccgcgtggtccggcacgttttctgcctctgcctggtctgcctccggcacctccggaacctccgggtattctggcaccgcagcctccggatgttggtagcagcgatccgctgagcatggtgggtcctagccagggtcgtagcccgagctatgcaagcggtagcggtagcattgaaggtcgtcatggtgaaggcacctttaccagcgatctgagcaaacaaatggaagaagaagcagttcgtctgtttattgaatggctgaaaaatggtggtccgagcagtggtgcacctcctccgagctaa

SEQ ID NO: 34 - DNA, 1010 bp, Artificial Sequence
  CR9444
tgggacaccaccatcatcatcatggtagcctgcaggatagcgaagttaatcaagaagcaaaaccggaagttaaaccggaagtgaaaccggaaacccatattaatctgaaagttagtgatggcagcagcgaaattttctttaaaatcaaaaaaaccacaccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcccatcgtgaacaaattggtggtcatccgattccggatagcagtccgctgctgcagtttggtggtcaggttcgtcagcgttatctgtataccgatgatgcacagcagaccgaagcacatctggaaattcgtgaagatggcaccgttggtggtgcagcagatcagagtccggaaagcctgctgcagctgaaagcactgaaaccgggtgttattcagattctgggtgttaaaaccagccgttttctgtgtcagcgtccggatggtgcactgtatggtagtctgcattttgatccggaagcatgtagctttcgtgaactgctgctggaagatggttataatgtttatcagagtgaagcacatggtctgccgctgcatctgcctggtaataaaagtccgcatcgtgatccggcaccgcgtggtccggcacgttttctgcctctgcctggtctgcctccggcacctccggaacctccgggtattctggcaccgcagcctccggatgttggtagcagcgatccgctgagcatggtgggtcctagccagggtcgtagcccgagctatgcaagcggtagcggtagcattgaaggtcagcatggtgaaggcacctttaccagcgatctgagcaaacaaatggaagaagaagcagttcgtctgtttattgaatggctgaaaaatggtggtccgagcagtggtgcacctcctccgagctaa

SEQ ID NO: 35 - DNA, 999 bp, Artificial Sequence
  CR9445
atgggacaccaccatcatcatcatggtagcctgcaggatagcgaagttaatcaagaagcaaaaccggaagttaaaccggaagtgaaaccggaaacccatattaatctgaaagttagtgatggcagcagcgaaattttctttaaaatcaaaaaaaccacaccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcccatcgtgaacaaattggtggtcatggtgaaggcacctttaccagcgatctgagcaaacaaatggaagaagaagcagttcgtctgtttattgaatggctgaaaaatggtggtccgagcagtggtgcacctcctccgagcattgaaggtcagcatccgattccggatagcagtccgctgctgcagtttggtggtcaggttcgtcagcgttatctgtataccgatgatgcacagcagaccgaagcacatctggaaattcgtgaagatggcaccgttggtggtgcagcagatcagagtccggaaagcctgctgcagctgaaagcactgaaaccgggtgttattcagattctgggtgttaaaaccagccgttttctgtgtcagcgtccggatggtgcactgtatggtagtctgcattttgatccggaagcatgtagctttcgtgaactgctgctggaagatggttataatgtttatcagagcgaagcacatggtctgcctctgcatctgcctggtaataaaagtccgcatcgtgatccggcaccgcgtggtccggcacgttttctgccgctgcctggtctgcctccggcacctccggaacctccgggtattctggcaccgcagcctccggatgttggtagcagcgatccgctgagcatggttggtccgagccagggtcgtagcccgagctatgcaagctaa

SEQ ID NO: 36 - DNA, 1008 bp, Artificial Sequence
  CR9446
atgggacaccaccatcatcatcatggtagcctgcaggatagcgaagttaatcaagaagcaaaaccggaagttaaaccggaagtgaaaccggaaacccatattaatctgaaagttagtgatggcagcagcgaaattttctttaaaatcaaaaaaaccacaccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcccatcgtgaacaaattggtggtcatggtgaaggcacctttaccagcgatctgagcaaacaaatggaagaagaagcagttcgtctgtttattgaatggctgaaaaatggtggtccgagcagtggtgcacctcctccgagcgcacctgccagccctgcaagccatccgattccggatagcagtccgctgctgcagtttggtggtcaggttcgtcagcgttatctgtataccgatgatgcacagcagaccgaagcacatctggaaattcgtgaagatggcaccgttggtggtgcagcagatcagagtccggaaagcctgctgcagctgaaagcactgaaaccgggtgttattcagattctgggtgttaaaaccagccgttttctgtgtcagcgtccggatggtgcactgtatggtagtctgcattttgatccggaagcatgtagctttcgtgaactgctgctggaagatggttataatgtttatcagagcgaagcacatggtctgcctctgcatctgcctggtaataaaagtccgcatcgtgatccggcaccgcgtggtccggcacgttttctgccgctgcctggtctgcctccggcacctccggaacctccgggtattctggcaccgcagcctccggatgttggtagcagcgatccgctgagcatggttggtccgagccagggtcgtagcccgagctatgcaagctaa

SEQ ID NO: 37 - DNA, 1011 bp, Artificial Sequence
  CR9447
atgggacaccaccatcatcatcatggtagcctgcaggatagcgaagttaatcaagaagcaaaaccggaagttaaaccggaagtgaaaccggaaacccatattaatctgaaagttagtgatggcagcagcgaaattttctttaaaatcaaaaaaaccacaccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcccatcgtgaacaaattggtggtcatggtgaaggcacctttaccagcgatctgagcaaacaaatggaagaagaagcagttcgtctgtttattgaatggctgaaaaatggtggtccgagcagtggtgcacctcctccgagcgcaccggcaagctgtccggcaagccatccgattccggatagcagtccgctgctgcagtttggtggtcaggttcgtcagcgttatctgtataccgatgatgcacagcagaccgaagcacatctggaaattcgtgaagatggcaccgttggtggtgcagcagatcagagtccggaaagcctgctgcagctgaaagcactgaaaccgggtgttattcagattctgggtgttaaaaccagccgttttctgtgtcagcgtccggatggtgcactgtatggtagtctgcattttgatccggaagcatgtagctttcgtgaactgctgctggaagatggttataatgtttatcagagcgaagcacatggtctgcctctgcatctgcctggtaataaaagtccgcatcgtgatccggcaccgcgtggtccggcacgttttctgccgctgcctggtctgcctccggcacctccggaacctccgggtattctggcaccgcagcctccggatgttggtagcagcgatccgctgagcatggttggtccgagccagggtcgtagcccgagctatgcaagctaa

SEQ ID NO: 38 - DNA, 999 bp, Artificial Sequence
  CR9448
atgggacaccaccatcatcatcatggtagcctgcaggatagcgaagttaatcaagaagcaaaaccggaagttaaaccggaagtgaaaccggaaacccatattaatctgaaagttagtgatggcagcagcgaaattttctttaaaatcaaaaaaaccacaccgctgcgtcgtctgatggaagcatttgcaaaacgtcagggtaaagaaatggatagcctgcgttttctgtatgatggtattcgtattcaggcagatcaggcaccggaagatctggatatggaagataatgatattattgaagcccatcgtgaacaaattggtggtcatggtgaaggcacctttaccagcgatctgagcaaacaaatggaagaagaagcagttcgtctgtttattgaatggctgaaaaatggtggtccgagcagtggtgcacctcctccgagcggtagcggtagccatccgattccggatagcagtccgctgctgcagtttggtggtcaggttcgtcagcgttatctgtataccgatgatgcacagcagaccgaagcacatctggaaattcgtgaagatggcaccgttggtggtgcagcagatcagagtccggaaagcctgctgcagctgaaagcactgaaaccgggtgttattcagattctgggtgttaaaaccagccgttttctgtgtcagcgtccggatggtgcactgtatggtagtctgcattttgatccggaagcatgtagctttcgtgaactgctgctggaagatggttataatgtttatcagagcgaagcacatggtctgcctctgcatctgcctggtaataaaagtccgcatcgtgatccggcaccgcgtggtccggcacgttttctgccgctgcctggtctgcctccggcacctccggaacctccgggtattctggcaccgcagcctccggatgttggtagcagcgatccgctgagcatggttggtccgagccagggtcgtagcccgagctatgcaagctaa

SEQ ID NO: 39 - protein, 336 aa, Artificial Sequence
  His-SUMO-FGF21-GSGSIEGR-Exenatide
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYASGSGSIEGRHGEGTFTSDLSKQMEEEAVRLFIEWLKNGGPSSGAPPPS

SEQ ID NO: 40 - protein, 336 aa, Artificial Sequence
  His-SUMO-FGF21-GSGSIEGQ-Exenatide
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYASGSGSIEGQHGEGTFTSDLSKQMEEEAVRLFIEWLKNGGPSSGAPPPS

SEQ ID NO: 41 - protein, 333 aa, Artificial Sequence
  His-SUMO-Exenatide-IEGQ-FGF21
MMGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPPSIEGQHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 42 - protein, 336 aa, Artificial Sequence
  His-SUMO-Exenatide-APASPAS-FGF21
MMGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPPSAPASPASHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 43 - protein, 336 aa, Artificial Sequence
  His-SUMO-Exenatide-APASCPAS-FGF21
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPPSAPASCPASHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 44 - protein, 332 aa, Artificial Sequence
  His-SUMO-Exenatide-GSGS-FGF21
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGHGEGTFTSDLSKQMEEEAVRLFIEWLKNGGPSSGAPPPSGSGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 45 - protein, 228 aa, Artificial Sequence
  FGF21-GSGSIEGR-Exenatide
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYASGSGSIEGRHGEGTFTSDLSKQMEEEAVRLFIEWLKNGGPSSGAPPPS

SEQ ID NO: 46 - protein, 228 aa, Artificial Sequence
  FGF21-GSGSIEGQ-Exenatide
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYASGSGSIEGQHGEGTFTSDLSKQMEEEAVRLFIEWLKNGGPSSGAPPPS

SEQ ID NO: 47 - protein, 224 aa, Artificial Sequence
  Exenatide-IEGQ-FGF21
HGEGTFTSDLSKQMEEEAVRLFIEWLKNGGPSSGAPPPSIEGQHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 48 - protein, 227 aa, Artificial Sequence
  Exenatide-APASPAS-FGF21
HGEGTFTSDLSKQMEEEAVRLFIEWLKNGGPSSGAPPPSAPASPASHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 49 - protein, 228 aa, Artificial Sequence
  Exenatide-APASCPAS-FGF21
HGEGTFTSDLSKQMEEEAVRLFIEWLKNGGPSSGAPPPSAPASCPASHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 50 - protein, 224 aa, Artificial Sequence
  Exenatide-GSGS-FGF21
HGEGTFTSDLSKQMEEEAVRLFIEWLKNGGPSSGAPPPSGSGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 51 - protein, 270 aa, Artificial Sequence
  Exenatide-GG-ABD-GG-FGF21
HGEGTFTSDLSKQMEEEAVRLFIEWLKNGGPSSGAPPPSGGLAEAKVLANRELDKYGVSDYYKNLINNAKTVEGVKALIDEILAALPGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 52 - protein, 276 aa, Artificial Sequence
  Exenatide-GGGGS-ABD-GGGGS-FGF21
HGEGTFTSDLSKQMEEEAVRLFIEWLKNGGPSSGAPPPSGGGGSLAEAKVLANRELDKYGVSDYYKNLINNAKTVEGVKALIDEILAALPGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 53 - protein, 268 aa, Artificial Sequence
  Exenatide-FGF21-GG-ABD
HGEGTFTSDLSKQMEEEAVRLFIEWLKNGGPSSGAPPPSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYASGGLAEAKVLANRELDKYGVSDYYKNLINNAKTVEGVKALIDEILAALP

SEQ ID NO: 54 - protein, 271 aa, Artificial Sequence
  Exenatide-FGF21-GGGGS-ABD
HGEGTFTSDLSKQMEEEAVRLFIEWLKNGGPSSGAPPPSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYASGGGGSLAEAKVLANRELDKYGVSDYYKNLINNAKTVEGVKALIDEILAALP

SEQ ID NO: 55 - protein, 451 aa, Artificial Sequence
  Exenatide-FGF21-GG-ABD-GG-FGF21
HGEGTFTSDLSKQMEEEAVRLFIEWLKNGGPSSGAPPPSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYASGGLAEAKVLANRELDKYGVSDYYKNLINNAKTVEGVKALIDEILAALPGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 56 - protein, 457 aa, Artificial Sequence
  Exenatide-FGF21-GGGGS-ABD-GGGGS-FGF21
HGEGTFTSDLSKQMEEEAVRLFIEWLKNGGPSSGAPPPSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYASGGGGSLAEAKVLANRELDKYGVSDYYKNLINNAKTVEGVKALIDEILAALPGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 57 - protein, 239 aa, Artificial Sequence
  Exenatide-GGGGS-His-GGGGS-FGF21
HGEGTFTSDLSKQMEEEAVRLFIEWLKNGGPSSGAPPPSGGGGSHAHGHGHAHGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 58 - protein, 287 aa, Artificial Sequence
  Exenatide-GGGGS-His-GGGGS-ABD-GG-FGF21
HGEGTFTSDLSKQMEEEAVRLFIEWLKNGGPSSGAPPPSGGGGSHAHGHGHAHGGGGSLAEAKVLANRELDKYGVSDYYKNLINNAKTVEGVKALIDEILAALPGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 59 - protein, 221 aa, Artificial Sequence
  Exenatide-(B)0-1000-FGF21 mutein-Cys
HGEGTFTSDLSKQMEEEAVRLFIEWLKNGGPSSGAPPPSBHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAC

SEQ ID NO: 60 - protein, 221 aa, Artificial Sequence
  Exenatide-(B)0-1000-FGF21 mutein-Lys
HGEGTFTSDLSKQMEEEAVRLFIEWLKNGGPSSGAPPPSBHPIPDSSPLLQFGGQVRQRYLYTDDAQQTEAHLEIREDGTVGGAADQSPESLLQLRALRPGVIQILGVRTSRFLCQRPDGALYGSLHFDPEACSFRELLLEDGYNVYQSEAHGLPLHLPGNRSPHRDPKPRGPARFLPLPGLPPAPPEPPGILAPQPPDVGSSDPLSMVGPSQGRSPSYAS

SEQ ID NO: 61 - protein, 243 aa, Artificial Sequence
  Exenatide-GG-Cys-(G)21-FGF21
HGEGTFTSDLSKQMEEEAVRLFIEWLKNGGPSSGAPPPSGGCGGGGGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 62 - protein, 243 aa, Artificial Sequence
  Exenatide-GG-Lys-(G)21-FGF21
HGEGTFTSDLSKQMEEEAVRLFIEWLKNGGPSSGAPPPSGGKGGGGGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 63 - protein, 451 aa, Artificial Sequence
  Exenatide-IgG 1 Asp103-Lys329-FGF21
HGEGTFTSDLSKQMEEEAVRLFIEWLKNGGPSSGAPPPSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 64 - protein, 434 aa, Artificial Sequence
  Exenatide-IgG1 Pro120-Lys329-FGF21
HGEGTFTSDLSKQMEEEAVRLFIEWLKNGGPSSGAPPPSGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 65 - protein, 434 aa, Artificial Sequence
  Exenatide-IgG1 Pro120-Lys329 mutated-FGF21
HGEGTFTSDLSKQMEEEAVRLFIEWLKNGGPSSGAPPPSGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFELKSALTVDKSRWQQGNVFSCSVMHEALHNHYTQKSLSLSPGKGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 66 - protein, 327 aa, Artificial Sequence
  Exenatide-IgG1 Pro120-Lys222-FGF21
HGEGTFTSDLSKQMEEEAVRLFIEWLKNGGPSSGAPPPSGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 67 - DNA, 835 bp, Artificial Sequence
  Exenatide-GGGGS-ABD-GGGGS-FGF21
cacggcgagggcaccttcaccagcgacctgagcaagcagatggaagaggaagccgtgcggctgttcatcgagtggctgaagaatggcggccctagctctggcgcccctccaccttctggcggcggaggatctctggccgaagccaaggtgctggccaacagagagctggataagtacggcgtgtccgactactacaagaacctgatcaacaacgccaagaccgtggaaggcgtgaaggccctgatcgacgagattctggctgccctgcctggcggagggggctctcatcctatccctgatagcagccccctgctgcagtttggcggacaagtgcggcagagatacctgtacaccgacgacgcccagcagaccgaggcccacctggaaatcagagaagatggcaccgtgggcggagccgccgatcagtctcctgaatctctgctgcagctgaaagccctgaagcccggcgtgatccagatcctgggcgtgaaaaccagccggttcctgtgccagaggcctgacggcgccctgtatggcagcctgcactttgatcctgaggcctgcagctttagagagctgctgctggaggacggctacaacgtgtaccagtctgaggcccacggcctgcccctgcatctgcctggaaacaagagcccccacagagatcccgcccctagaggccctgccagattcctgcctctgcccggactgcctcctgcccctcctgaacctcctggaattctggccccccagcctcctgatgtgggcagctctgatcccctgagcatggtgggacctagccagggcagaagccctagctacgccagctaatgaa

SEQ ID NO: 68 - DNA, 819 bp, Artificial Sequence
  Exenatide-FGF21-GGGGS-ABD
cacggcgagggcaccttcaccagcgacctgagcaagcagatggaagaggaagccgtgcggctgttcatcgagtggctgaagaatggcggccctagctctggcgcccctcctccttcacaccccatccctgatagcagccccctgctgcagtttggcggacaagtgcggcagagatacctgtacaccgacgacgcccagcagaccgaggcccacctggaaatcagagaagatggcaccgtgggcggagccgccgatcagtctcctgaatctctgctgcagctgaaggccctgaagcccggcgtgatccagatcctgggcgtgaaaaccagccggttcctgtgccagaggcctgacggcgccctgtatggcagcctgcactttgatcctgaggcctgcagcttcagagagctgctgctggaggacggctacaacgtgtaccagtctgaggcccacggcctgcccctgcatctgcctggaaacaagagcccccacagagatcccgcccctagaggccctgccagattcctgccactgcctggactgcctccagcccctcctgagcctcctggaattctggctccccagcctcctgatgtgggcagcagcgatcctctgagcatggtgggacctagccagggcagaagccctagctacgcttctggcggcggaggatctctggccgaggctaaggtgctggccaatagagagctggataagtacggcgtgtccgactactacaagaacctgatcaacaacgccaagaccgtggaaggcgtgaaagccctgatcgacgagatcctggccgccctgccctaatga

SEQ ID NO: 69 - DNA, 1377 bp, Artificial Sequence
  Exenatide-FGF21-GGGGS-ABD-GGGGS-FGF21
cacggcgagggcaccttcaccagcgacctgagcaagcagatggaagaggaagccgtgcggctgttcatcgagtggctgaagaatggcggccctagctctggcgcccctcctccttcacaccccatccctgatagcagccccctgctgcagtttggcggacaagtgcggcagagatacctgtacaccgacgacgcccagcagaccgaggcccacctggaaatcagagaagatggcaccgtgggcggagccgccgatcagtctcctgaatctctgctgcagctgaaggccctgaagcccggcgtgatccagatcctgggcgtgaaaaccagccggttcctgtgccagaggcctgacggcgccctgtatggcagcctgcactttgatcctgaggcctgcagcttcagagagctgctgctggaggacggctacaacgtgtaccagtctgaggcccacggcctgcccctgcatctgcctggaaacaagagcccccacagagatcccgcccctagaggccctgccagattcctgccactgcctggactgcctccagcccctcctgagcctcctggaattctggctccccagcctcctgatgtgggcagcagcgatcctctgagcatggtgggacctagccagggcagaagccctagctacgcttctggcggcggaggatctctggccgaggctaaggtgctggccaatagagagctggataagtacggcgtgtccgactactacaagaacctgatcaacaacgccaagaccgtggaaggcgtgaaagccctgatcgacgagatcctggctgctctgccaggcggagggggatctcaccctatcccagattctagtcctctgctgcagttcggaggccaagtgcgccagcggtatctgtatactgatgatgctcagcagacagaagctcatctggaaattcgcgaggacggcacagtgggaggcgctgctgatcagagcccagaaagcctgctgcagctgaaagctctgaaacctggcgtgatccagattctgggagtgaaaacatcccgctttctgtgtcagcgccccgatggcgctctgtacggctctctgcacttcgaccccgaagcctgctccttccgggaactgctgctggaagatgggtataatgtgtatcagagcgaagcccatggactgcctctgcatctgcccggcaacaaatccccccatagggaccctgccccaaggggaccagctagatttctgcctctgcccggcctgccaccagctccaccagaacctccaggcattctggcacctcagcccccagacgtgggaagctctgaccctctgtctatggtgggcccctctcagggcagatctcccagctacgccagctaatga

SEQ ID NO: 70 - DNA, 810 bp, Artificial Sequence
  Exenatide-GG-ABD-GG-FGF21
cacggcgagggcaccttcaccagcgacctgagcaagcagatggaggaggaggccgtgagactgttcatcgagtggctgaagaacggcggccccagcagcggcgcccccccccccagcggcggcctggccgaggccaaggtgctggccaacagagagctggacaagtacggcgtgagcgactactacaagaacctgatcaacaacgccaagaccgtggagggcgtgaaggccctgatcgacgagatcctggccgccctgcccggcggccaccccatccccgacagcagccccctgctgcagttcggcggccaggtgagacagagatacctgtacaccgacgacgcccagcagaccgaggcccacctggagatcagagaggacggcaccgtgggcggcgccgccgaccagagccccgagagcctgctgcagctgaaggccctgaagcccggcgtgatccagatcctgggcgtgaagaccagcagattcctgtgccagagacccgacggcgccctgtacggcagcctgcacttcgaccccgaggcctgcagcttcagagagctgctgctggaggacggctacaacgtgtaccagagcgaggcccacggcctgcccctgcacctgcccggcaacaagagcccccacagagaccccgcccccagaggccccgccagattcctgcccctgcccggcctgccccccgccccccccgagccccccggcatcctggccccccagccccccgacgtgggcagcagcgaccccctgagcatggtgggccccagccagggcagaagccccagctacgccagc

SEQ ID NO: 71 - DNA, 804 bp, Artificial Sequence
  Exenatide-FGF21-GG-ABD
cacggcgagggcaccttcaccagcgacctgagcaagcagatggaggaggaggccgtgagactgttcatcgagtggctgaagaacggcggccccagcagcggcgcccccccccccagccaccccatccccgacagcagccccctgctgcagttcggcggccaggtgagacagagatacctgtacaccgacgacgcccagcagaccgaggcccacctggagatcagagaggacggcaccgtgggcggcgccgccgaccagagccccgagagcctgctgcagctgaaggccctgaagcccggcgtgatccagatcctgggcgtgaagaccagcagattcctgtgccagagacccgacggcgccctgtacggcagcctgcacttcgaccccgaggcctgcagcttcagagagctgctgctggaggacggctacaacgtgtaccagagcgaggcccacggcctgcccctgcacctgcccggcaacaagagcccccacagagaccccgcccccagaggccccgccagattcctgcccctgcccggcctgccccccgccccccccgagccccccggcatcctggccccccagccccccgacgtgggcagcagcgaccccctgagcatggtgggccccagccagggcagaagccccagctacgccagcggcggcctggccgaggccaaggtgctggccaacagagagctggacaagtacggcgtgagcgactactacaagaacctgatcaacaacgccaagaccgtggagggcgtgaaggccctgatcgacgagatcctggccgccctgccc

SEQ ID NO: 72 - DNA, 1353 bp, Artificial Sequence
  Exenatide-FGF21-GG-ABD-GG-FGF21
cacggcgagggcaccttcaccagcgacctgagcaagcagatggaggaggaggccgtgagactgttcatcgagtggctgaagaacggcggccccagcagcggcgcccccccccccagccaccccatccccgacagcagccccctgctgcagttcggcggccaggtgagacagagatacctgtacaccgacgacgcccagcagaccgaggcccacctggagatcagagaggacggcaccgtgggcggcgccgccgaccagagccccgagagcctgctgcagctgaaggccctgaagcccggcgtgatccagatcctgggcgtgaagaccagcagattcctgtgccagagacccgacggcgccctgtacggcagcctgcacttcgaccccgaggcctgcagcttcagagagctgctgctggaggacggctacaacgtgtaccagagcgaggcccacggcctgcccctgcacctgcccggcaacaagagcccccacagagaccccgcccccagaggccccgccagattcctgcccctgcccggcctgccccccgccccccccgagccccccggcatcctggccccccagccccccgacgtgggcagcagcgaccccctgagcatggtgggccccagccagggcagaagccccagctacgccagcggcggcctggccgaggccaaggtgctggccaacagagagctggacaagtacggcgtgagcgactactacaagaacctgatcaacaacgccaagaccgtggagggcgtgaaggccctgatcgacgagatcctggccgccctgcccggcggccaccccatccccgacagcagccccctgctgcagttcggcggccaggtgagacagagatacctgtacaccgacgacgcccagcagaccgaggcccacctggagatcagagaggacggcaccgtgggcggcgccgccgaccagagccccgagagcctgctgcagctgaaggccctgaagcccggcgtgatccagatcctgggcgtgaagaccagcagattcctgtgccagagacccgacggcgccctgtacggcagcctgcacttcgaccccgaggcctgcagcttcagagagctgctgctggaggacggctacaacgtgtaccagagcgaggcccacggcctgcccctgcacctgcccggcaacaagagcccccacagagaccccgcccccagaggccccgccagattcctgcccctgcccggcctgccccccgccccccccgagccccccggcatcctggccccccagccccccgacgtgggcagcagcgaccccctgagcatggtgggccccagccagggcagaagccccagctacgccagc

SEQ ID NO: 73 - DNA, 720 bp, Artificial Sequence
  Exenatide-GGGGS-His-GGGGS-FGF21
cacggcgagggcaccttcaccagcgacctgagcaagcagatggaagaggaagccgtgcggctgttcatcgagtggctgaagaatggcggccctagctctggcgcccctccaccttctggcggcggaggatctcatgcccacggacacggacatgctcatggcggaggcggctctcaccccatccctgatagtagccccctgctgcagtttggcggacaagtgcggcagagatacctgtacaccgacgacgcccagcagaccgaggcccacctggaaatcagagaagatggcaccgtgggcggagccgccgatcagtctcctgaatctctgctgcagctgaaggccctgaagcccggcgtgatccagatcctgggcgtgaaaaccagccggttcctgtgccagaggcctgacggcgccctgtatggcagcctgcactttgatcctgaggcctgcagcttcagagagctgctgctggaggacggctacaacgtgtaccagtctgaggcccacggcctgcccctgcatctgcctggaaacaagagcccccacagagatcccgcccctagaggccctgccagattcctgccactgcctggactgcctccagcccctcctgagcctcctggaattctggctccccagcctcctgatgtgggcagcagcgatcctctgagcatggtgggacctagccagggcagaagccctagctacgccagctaa

SEQ ID NO: 74 - DNA, 864 bp, Artificial Sequence
  Exenatide-GGGGS-His-GGGGS-ABD-GG-FGF21
cacggcgagggcaccttcaccagcgacctgagcaagcagatggaagaggaagccgtgcggctgttcatcgagtggctgaagaatggcggccctagctctggcgcccctccaccttctggcggcggaggatctcatgcccacggacacggacatgctcatggcggaggcggatctctggccgaggctaaggtgctggccaacagagagctggataagtacggcgtgtccgactactacaagaacctgatcaacaacgccaagaccgtggaaggcgtgaaggccctgatcgacgagattctggctgccctgcctggcggccaccctatccctgattcaagccccctgctgcagttcggcggacaagtgcggcagagatacctgtacaccgacgacgcccagcagaccgaggcccacctggaaatcagagaagatggcaccgtgggcggagccgccgatcagtctcctgaatctctgctgcagctgaaagccctgaagcccggcgtgatccagatcctgggcgtgaaaaccagccggttcctgtgccagaggcctgacggcgccctgtatggcagcctgcactttgatcctgaggcctgcagctttagagagctgctgctggaggacggctacaacgtgtaccagtctgaggcccacggcctgcccctgcatctgcctggaaacaagagcccccacagagatcccgcccctagaggccctgccagattcctgcctctgcccggactgcctcctgcccctcctgaacctcctggaattctggccccccagcctcctgatgtgggcagctctgatcccctgagcatggtgggacctagccagggcagaagccctagctacgccagctaa

SEQ ID NO: 75 - DNA, 729 bp, Artificial Sequence
  Exenatide-GG-Cys-(G)21-FGF21
cacggcgagggcaccttcaccagcgacctgagcaagcagatggaggaggaggccgtgagactgttcatcgagtggctgaagaacggcggccccagcagcggcgcccccccccccagcggcggctgcggcggcggcggcggcggcggcggcggcagcggcggcggcggcagcggcggcggcggcagccaccccatccccgacagcagccccctgctgcagttcggcggccaggtgagacagagatacctgtacaccgacgacgcccagcagaccgaggcccacctggagatcagagaggacggcaccgtgggcggcgccgccgaccagagccccgagagcctgctgcagctgaaggccctgaagcccggcgtgatccagatcctgggcgtgaagaccagcagattcctgtgccagagacccgacggcgccctgtacggcagcctgcacttcgaccccgaggcctgcagcttcagagagctgctgctggaggacggctacaacgtgtaccagagcgaggcccacggcctgcccctgcacctgcccggcaacaagagcccccacagagaccccgcccccagaggccccgccagattcctgcccctgcccggcctgccccccgccccccccgagccccccggcatcctggccccccagccccccgacgtgggcagcagcgaccccctgagcatggtgggccccagccagggcagaagccccagctacgccagc

SEQ ID NO: 76 - DNA, 729 bp, Artificial Sequence
  Exenatide-GG-Lys-(G)21-FGF21
cacggcgagggcaccttcaccagcgacctgagcaagcagatggaggaggaggccgtgagactgttcatcgagtggctgaagaacggcggccccagcagcggcgcccccccccccagcggcggcaagggcggcggcggcggcggcggcggcggcagcggcggcggcggcagcggcggcggcggcagccaccccatccccgacagcagccccctgctgcagttcggcggccaggtgagacagagatacctgtacaccgacgacgcccagcagaccgaggcccacctggagatcagagaggacggcaccgtgggcggcgccgccgaccagagccccgagagcctgctgcagctgaaggccctgaagcccggcgtgatccagatcctgggcgtgaagaccagcagattcctgtgccagagacccgacggcgccctgtacggcagcctgcacttcgaccccgaggcctgcagcttcagagagctgctgctggaggacggctacaacgtgtaccagagcgaggcccacggcctgcccctgcacctgcccggcaacaagagcccccacagagaccccgcccccagaggccccgccagattcctgcccctgcccggcctgccccccgccccccccgagccccccggcatcctggccccccagccccccgacgtgggcagcagcgaccccctgagcatggtgggccccagccagggcagaagccccagctacgccagc

SEQ ID NO: 77 - DNA, 1276 bp, Artificial Sequence
  Exenatide-GG-IgG 1 Asp103-Lys329-GG-FGF21
catggtgaaggcacctttaccagcgatctgagcaaacaaatggaagaagaagcagttcgcctgtttattgaatggctgaaaaatggtggtccgagcagtggtgcaccgcctccgagtggtggtgataaaacccatacctgtccgccttgtccggctccggaactgctgggtggtccgtcagtttttctgtttccgcctaaaccgaaagataccctgatgattagccgtacaccggaagtgacctgtgttgttgttgatgttagccatgaagatcctgaggtgaaatttaactggtatgttgatggtgtggaagtgcataatgcaaaaacaaaaccgcgtgaggaacagtataattcaacctatcgtgttgttagcgttctgaccgttctgcatcaggattggctgaatggtaaagaatacaaatgcaaagtgagcaacaaagcactgcctgcaccgattgaaaaaaccattagcaaagcaaaaggtcagcctcgtgaaccgcaggtttataccctgcctccgagccgtgatgaactgaccaaaaatcaggttagcctgacctgtctggtgaaaggtttttatccgagcgatattgcagttgaatgggaaagcaatggtcagccggaaaataactataaaaccacccctccggttctggatagtgatggtagctttttcctgtatagcaaactgaccgttgataaaagccgttggcagcagggtaatgtttttagctgtagcgttatgcatgaagccctgcataatcattatacccagaaaagcctgagcctgagtccgggtaaaggcggtcatccgattccggatagcagtccgctgctgcagtttggtggccaggttcgtcagcgttatctgtataccgatgatgcacagcagaccgaagcccatctggaaattcgtgaagatggcaccgttggtggtgcagcagatcagagtccggaaagcctgctgcagctgaaagcactgaaaccgggtgttattcagattctgggtgttaaaaccagccgctttctgtgtcagcgtccggatggtgcactgtatggtagtctgcattttgatccggaagcatgtagctttcgtgaactgctgctggaagatggttataatgtttatcagagcgaagcgcatggtctgccgctgcatctgcctggtaataaaagtccgcatcgtgatccggcaccgcgtggtccggcacgttttctgcctctgccaggtctgcctccggcacctcctgaaccgcctggtattctggcaccgc

SEQ ID NO: 78 - DNA, 1305 bp, Artificial Sequence
  Exenatide-GG-IgG1 Pro120-Lys329-GG-FGF21
catggtgaaggcacctttaccagcgatctgagcaaacaaatggaagaagaagcagttcgcctgtttattgaatggctgaaaaatggtggtccgagcagtggtgcaccgcctccgtcaggtggtccgtcagtttttctgtttccgcctaaaccgaaagataccctgatgattagccgtacaccggaagtgacctgtgttgttgttgatgttagccatgaagatcctgaggtgaaatttaactggtatgttgatggtgtggaagtgcataatgcaaaaacaaaaccgcgtgaggaacagtataattcaacctatcgtgttgttagcgttctgaccgttctgcatcaggattggctgaatggtaaagaatacaaatgcaaagtgagcaacaaagcactgcctgcaccgattgaaaaaaccattagcaaagcaaaaggtcagcctcgtgaaccgcaggtttataccctgcctccgagccgtgatgaactgaccaaaaatcaggttagcctgacctgtctggtgaaaggtttttatccgagcgatattgcagttgaatgggaaagcaatggtcagccggaaaataactataaaaccacccctccggttctggatagtgatggtagctttttcctgtatagcaaactgaccgttgataaaagccgttggcagcagggtaatgtttttagctgtagcgttatgcatgaagccctgcataatcattatacccagaaaagcctgagcctgagtccgggtaaaggcggtcatccgattccggatagcagtccgctgctgcagtttggtggccaggttcgtcagcgttatctgtataccgatgatgcacagcagaccgaagcccatctggaaattcgtgaagatggcaccgttggtggtgcagcagatcagagtccggaaagcctgctgcagctgaaagcactgaaaccgggtgttattcagattctgggtgttaaaaccagccgctttctgtgtcagcgtccggatggtgcactgtatggtagtctgcattttgatccggaagcatgtagctttcgtgaactgctgctggaagatggttataatgtttatcagagcgaagcgcatggtctgccgctgcatctgcctggtaataaaagtccgcatcgtgatccggcaccgcgtggtccggcacgttttctgcctctgccaggtctgcctccggcacctcctgaaccgcctggtattctggcaccgcagcctccggatgttggtagcagcgatccgctgagcatggtgggtccgtcacagggtcgtagcccgagctatgcaagctaa

SEQ ID NO: 79 - protein, 227 aa, Artificial Sequence
  Fc fragment 1: IgG 1 Asp103-Lys329
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 210 aa, Artificial Sequence
  Fc fragment 2: IgG1 Pro120-Lys329
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 210 aa, Artificial Sequence
  Fc fragment 3: IgG1 Pro120-Lys329 mutated
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFELKSALTVDKSRWQQGNVFSCSVMHEALHNHYTQKSLSLSPGK

SEQ ID NO: 82 - protein, 105 aa, Artificial Sequence
  Fc fragment 4: IgG1 Pro120-Lys222
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 83 - protein, 231 aa, Artificial Sequence
  GG-(IgG 1 Asp103-Lys329)-GG
GGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGG

SEQ ID NO: 84 - protein, 214 aa, Artificial Sequence
  GG-(IgG1 Pro120-Lys329)-GG
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGG

SEQ ID NO: 85 - protein, 214 aa, Artificial Sequence
  GG-(IgG1 Pro120-Lys329 mutated)-GG
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFELKSALTVDKSRWQQGNVFSCSVMHEALHNHYTQKSLSLSPGKGG

SEQ ID NO: 86 - protein, 107 aa, Artificial Sequence
  GG-(IgG1 Pro120-Lys222)-GG
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGG

SEQ ID NO: 87 - protein, 46 aa, Artificial Sequence
  Albumin-Binding Domain (ABD)
LAEAKVLANRELDKYGVSDYYKNLINNAKTVEGVKALIDEILAALP

SEQ ID NO: 88 - protein, 50 aa, Artificial Sequence
  GG-Albumin-Binding Domain-GG
GGLAEAKVLANRELDKYGVSDYYKNLINNAKTVEGVKALIDEILAALPGG

SEQ ID NO: 89 - protein, 56 aa, Artificial Sequence
  GGGGS-Albumin-Binding Domain-GGGGS
GGGGSLAEAKVLANRELDKYGVSDYYKNLINNAKTVEGVKALIDEILAALPGGGGS

SEQ ID NO: 90 - protein, 585 aa, Homo sapiens [sequence omitted]

SEQ ID NO: 91 - protein, 623 aa, Artificial Sequence
  Human Serum Albumine (HSA) with linker (GG[GGGGS]3)A-HSA-GG[GGGGS]3)A)
GGGGGGGSGGGGSGGGGSADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGGGGSGGGGSGGGGSA

SEQ ID NO: 92 - protein, 14 aa, Artificial Sequence
  Sequence with multiple His-residues 1
HAHGHGHAHGGGGS

SEQ ID NO: 93 - protein, 9 aa, Artificial Sequence
  Sequence with multiple His-residues 2
HAHGHGHAH

SEQ ID NO: 94 - protein, 181 aa, Artificial Sequence; identical to SEQ ID NO: 3
  FGF21 (without signal sequence) based linker

SEQ ID NO: 95 - protein, 20 aa, Artificial Sequence
  PASylation Sequence 1
ASPAAPAPASPAAPAPSAPA

SEQ ID NO: 96 - protein, 20 aa, Artificial Sequence
  PASylation Sequence 2
AAPASPAPAAPSAPAPAAPS

SEQ ID NO: 97 - protein, 20 aa, Artificial Sequence
  PASylation Sequence 3
APSSPSPSAPSSPSPASPSS

SEQ ID NO: 98 - protein, 20 aa, Artificial Sequence
  PASylation Sequence 4
SAPSSPSPSAPSSPSPASPS

SEQ ID NO: 99 - protein, 20 aa, Artificial Sequence
  PASylation Sequence 5
SSPSAPSPSSPASPSPSSPA

SEQ ID NO: 100 - protein, 24 aa, Artificial Sequence
  PASylation Sequence 6
AASPAAPSAPPAAASPAAPSAPPA

SEQ ID NO: 101 - protein, 20 aa, Artificial Sequence
  PASylation Sequence 7
ASAAAPAAASAAASAPSAAA

SEQ ID NO: 102 - protein, 182 aa, Artificial Sequence
  FGF-21 mutein G + FGF-21 (without signal sequence)
GHPIPDSSPLLQPGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 103 - DNA, 1305 bp, Artificial Sequence
  Exenatide-GG-IgG1 Pro120-Lys329 mutated-GG-FGF21
catggtgaaggcacctttaccagcgatctgagcaaacaaatggaagaagaagcagttcgcctgtttattgaatggctgaaaaatggtggtccgagcagtggtgcaccgcctccgtcaggtggtccgtcagtttttctgtttccgcctaaaccgaaagataccctgatgattagccgtacaccggaagtgacctgtgttgttgttgatgttagccatgaagatcctgaggtgaaatttaactggtatgttgatggtgtggaagtgcataatgcaaaaacaaaaccgcgtgaggaacagtataattcaacctatcgtgttgttagcgttctgaccgttctgcatcaggattggctgaatggtaaagaatacaaatgcaaagtgagcaacaaagcactgcctgcaccgattgaaaaaaccattagcaaagcaaaaggtcagcctcgtgaaccgcaggtttataccagccctccgagccgtgatgaactgaccaaaaatcaggttagtctgcgttgtcatgtgaaaggtttttatccgagcgatattgcagttgaatgggaaagcaatggtcagccggaaaataactataaaaccaccaaaccggttctggattcagatggttcatttgaactgaaaagcgcactgaccgttgataaaagccgttggcagcagggtaatgtttttagctgtagcgttatgcatgaagccctgcataatcattatacccagaaaagcctgagcctgagtccgggtaaaggcggtcatccgattccggatagcagtccgctgctgcagtttggtggccaggttcgtcagcgttatctgtataccgatgatgcacagcagaccgaagcccatctggaaattcgtgaagatggcaccgttggtggtgcagcagatcagagtccggaaagcctgctgcagctgaaagcactgaaaccgggtgttattcagattctgggtgttaaaaccagccgctttctgtgtcagcgtccggatggtgcactgtatggtagtctgcattttgatccggaagcatgtagctttcgtgaactgctgctggaagatggttataatgtttatcagagcgaagcgcatggtctgccgctgcatctgcctggtaataaaagtccgcatcgtgatccggcaccgcgtggtccggcacgttttctgcctctgccaggtctgcctccggcacctcctgaaccgcctggtattctggcaccgcagcctccggatgttggtagcagcgatccgctgagcatggtgggtccgtcacagggtcgtagcccgagctatgcaagctaa

SEQ ID NO: 104 - DNA, 984 bp, Artificial Sequence
  Exenatide-GG-IgG1 Pro120-Lys222-GG-FGF21
catggtgaaggcacctttaccagcgatctgagcaaacaaatggaagaagaagcagttcgcctgtttattgaatggctgaaaaatggtggtccgagcagtggtgcaccgcctccgtcaggtggtccgtcagtttttctgtttccgcctaaaccgaaagataccctgatgattagccgtacaccggaagtgacctgtgttgttgttgatgttagccatgaagatcctgaggtgaaatttaactggtatgttgatggtgtggaagtgcataatgcaaaaacaaaaccgcgtgaggaacagtataattcaacctatcgtgttgttagcgttctgaccgttctgcatcaggattggctgaatggtaaagaatacaaatgcaaagtgagcaacaaagcactgcctgcaccgattgaaaaaaccattagcaaagcaaaaggtggtcatccgattccggatagcagtccgctgctgcagtttggtggccaggttcgtcagcgttatctgtataccgatgatgcacagcagaccgaagcccatctggaaattcgtgaagatggcaccgttggtggtgcagcagatcagagtccggaaagcctgctgcagctgaaagcactgaaaccgggtgttattcagattctgggtgttaaaaccagccgctttctgtgtcagcgtccggatggtgcactgtatggtagtctgcattttgatccggaagcatgtagctttcgtgaactgctgctggaagatggttataatgtttatcagagcgaagcgcatggtctgccgctgcatctgcctggtaataaaagtccgcatcgtgatccggcaccgcgtggtccggcacgttttctgcctctgccaggtctgcctccggcacctcctgaaccgcctggtattctggcaccgcagcctccggatgttggtagcagcgatccgctgagcatggtgggtccgtcacagggtcgtagcccgagctatgcaagctaa

(19) United States
(12) Patent Application Publication
(10) Pub. No.: US 2016/0194371 A1
(43) Pub. Date: Jul. 7, 2016

(54) Fusion proteins comprising FGF-21 and GLP-1R agonist

(71) Applicant: Sanofi, Paris (FR)

(72) Inventors: Oliver Boscheinen, Frankfurt am Main (DE); Matthias Dreyer, Frankfurt am Main (DE); Paul Habermann, Frankfurt am Main (DE); Hans-Ludwig Schaefer, Frankfurt am Main (DE); Mark Sommerfeld, Frankfurt am Main (DE); Thomas Langer, Frankfurt am Main (DE)

(21) Appl. No.: 14/965,841

(22) Filed: Dec. 10, 2015

Related U.S. Application Data
(63) Continuation of application No. 14/019,735, filed on Sep. 6, 2013, now abandoned.

(30) Foreign Application Priority Data
Sep. 7, 2012 (EP) 12306072.5

(51) Int. Cl. C07K 14/605; C07K 14/575; C07K 14/50

(52) U.S. Cl. CPC C07K 14/605; C07K 14/50; C07K 14/575; C07K 2319/21; C07K 2319/50

(57) ABSTRACT

The invention is directed to a fusion protein comprising at least one FGF-21 (fibroblast growth factor-21) compound and at least one GLP-1R (glucagon-like peptide-1 receptor) agonist as well as to pharmaceutical compositions, medical uses and methods of treatment involving the fusion protein, particularly in the field of diabetes, dyslipidemia, obesity and/or adipositas.

The present invention is directed to FGF-21 fusion proteins as well as pharmaceutical compounds comprising the same, a pharmaceutical composition, uses and methods involving FGF fusion proteins, particularly or the treatment of at least one metabolic syndrome and/or atherosclerosis, in particular diabetes, dyslipidemia, obesity and/or adipositas.

### BACKGROUND

Diabetes mellitus is characterized by its clinical manifestations, namely the non-insulin-dependent or maturity onset form, also known as Type 2 diabetes, and the insulin-dependent or juvenile onset form, also known as Type 1 diabetes. The manifestations of clinical symptoms of Type 2 diabetes and the underlying obesity usually appear at an age over 40. In contrast, Type 1 diabetes usually shows a rapid onset of the disease, often before 30. The disease is a metabolic disorder in humans with a prevalence of approximately one percent in the general population, with one-fourth of these being Type 1 and three-fourths of these being Type 2 diabetes. Type 2 diabetes is a disease characterized by high-circulating blood glucose, insulin and corticosteroid levels.

Currently, there are various pharmacological approaches for the treatment of Type 2 diabetes, which may be utilized individually or in combination, and which act via different modes of action:

1) sulfonylurea stimulates insulin secretion;

2) biguanides (metformin) act by promoting glucose utilization, reducing hepatic glucose production and diminishing intestinal glucose output;

3) Glucagon-like peptide-1 receptor agonists (GLP-1 R agonists) known as the “incretin mimetics” acting as glucose-dependent insulin secretion by the pancreatic beta-cell, and slows gastric emptying.

4) oc-glucosidase inhibitors (acarbose, miglitol) slow down carbohydrate digestion and consequently absorption from the gut and reduce postprandial hyperglycemia;

5) thiazolidinediones (troglitazone) enhance insulin action, thus promoting glucose utilization in peripheral tissues; and

6) insulin stimulates tissue glucose utilization and inhibits hepatic glucose output.

However, most of the drugs have limited efficacy and do not address the most important problems, the declining beta-cell function and the associated obesity.

Type 1 diabetes results from an autoimmune destruction of insulin-producing beta cells of the pancreas and characteristically show very low or immeasurable plasma insulin with elevated glucagon. An immune response specifically directed against beta-cells leads to Type 1 diabetes because beta-cells secrete insulin. Current therapeutic regimens for Type 1 diabetes try to minimize hyperglycemia resulting from the lack of natural insulin.

Obesity is a chronic disease that is highly prevalent in modern society and is associated with numerous medical problems including diabetes mellitus, insulin resistance, hypertension, hypercholesterolemia, and coronary heart disease. It is further highly correlated with diabetes and insulin resistance, the latter of which is generally accompanied by hyperinsulinemia or hyperglycemia, or both. In addition, Type 2 diabetes is associated with a two to fourfold risk of coronary artery disease.

Fibroblast growth factor 21 (FGF21 or FGF-21) is a novel metabolic regulator produced primarily by the liver that exerts potent antidiabetic and lipid-lowering effects in animal models of obesity and type 2 diabetes mellitus. This hormone contributes to body weight regulation and is involved in the response to nutritional deprivation and ketogenic state in mice. The principal sites of metabolic actions of FGF-21 are adipose tissue, liver and pancreas. Experimental studies have shown improvements in diabetes compensation and dyslipidemia after FGF-21 administration in diabetic mice and primates (Dostalova et al. 2009). FGF-21 has been shown to stimulate glucose uptake in mouse 3T3-L1 adipocytes in the presence and absence of insulin, and to decrease fed and fasting blood glucose, triglycerides, and glucagon levels in ob/ob and db/db mice and 8 week old ZDF rats in a dose-dependent manner, thus, providing the basis for the use of FGF-21 as a therapy for treating diabetes and obesity (see e.g.

Fibroblast growth factors (FGFs) are polypeptides that are widely expressed in developing and adult tissues. The FGF family currently consists of twenty-three members, FGF-1 to FGF-23. The members of the FGF family are highly conserved in both gene structure and amino acid sequence between vertebrate species. There are 18 mammalian fibroblast growth factors (FGF1-FGF10 and FGF16-FGF23) which are grouped into 6 subfamilies based on differences in sequence homology and phylogeny. The numbered ‘FGFs’ that are unassigned to subfamilies—the FGF homologous factors (previously known as FGF11-FGF14)—have high sequence identity with the FGF family but do not activate FGF receptors (FGFRs) and are therefore not generally considered members of the FGF family.

While most FGFs act as local regulators of cell growth and differentiation, recent studies indicated that FGF-19 subfamily members including FGF-15/-19, FGF-21 and FGF-23 exert important metabolic effects by an endocrine fashion. The members of the FGF-19 subfamily regulate diverse physiological processes that are not affected by classical FGFs. The wide variety of metabolic activities of these endocrine factors include the regulation of the bile acid, carbohydrate and lipid metabolism as well as phosphate, calcium and vitamin D homeostasis (Tomlinson et al. 2002, Holt et al. 2003, Shimada et al. 2004, Kharitonenkov et al. 2005, Inagaki et al. 2005, Lundasen et al. 2006).

FGF-21 was originally isolated from mouse embryos. FGF-21 mRNA was most abundantly expressed in the liver, and to a lesser extent in the thymus (Nishimura et al. 2000). Human FGF-21 is highly identical (approximately 75% amino acid identity) to mouse FGF-21. Among human FGF family members, FGF-21 is the most similar (approximately 35% amino acid identity) to FGF19 (Nishimura et al. 2000). FGF-21 is free of the proliferative and tumorigenic effects (Kharitonenkov et al. 2005, Huang et al. 2006, Wente et al. 2006) that are typical for the majority of the members of FGF family (Ornitz and Itoh 2001, Nicholes et al. 2002, Eswarakumar et al. 2005).

The administration of FGF-21 to obese leptin-deficient ob/ob and leptin receptor-deficient db/db mice and obese ZDF rats significantly lowered blood glucose and triglycerides, decreased fasting insulin levels and improved glucose clearance during an oral glucose tolerance test. FGF-21 did not affect food intake or body weight/composition of diabetic or lean mice and rats over the course of 2 weeks of administration. Importantly, FGF-21 did not induce mitogenicity, hypoglycemia, or weight gain at any dose tested in diabetic or healthy animals or when overexpressed in transgenic mice (Kharitonenkov etal. 2005). FGF-21-overexpressing transgenic mice were resistant to diet-induced obesity.

The administration of FGF-21 to diabetic rhesus monkeys for 6 weeks reduced fasting plasma glucose, fructosamine, triglyceride, insulin and glucagone levels. Importantly, hypoglycemia was not observed during the study despite significant glucose-lowering effects. FGF-21 administration also significantly lowered LDL-cholesterol and increased HDL-cholesterol and, in contrast to mice (Kharitonenkov et al. 2005), slightly but significantly decreased body weight (Kharitonenkov et al. 2007).

Further information can be taken from the following references:
1. DOSTALOVA I. et al.: Fibroblast Growth Factor 21: A Novel Metabolic Regulator With Potential Therapeutic Properties in Obesity/Type 2 Diabetes Mellitus. Physiol Res 58: 1-7, 2009.
2. ESWARAKUMAR V. P. et al.: Cellular signaling by fibroblast growth factor receptors. Cytokine Growth Factor Rev 16: 139-149, 2005.
3. HOLT J. A. et al.: Definition of a novel growth factor-dependent signal cascade for the suppression of bile acid biosynthesis. Genes Dev 17: 1581-1591, 2003.
4. HUANG X. et al.: Forced expression of hepatocytespecific fibroblast growth factor 21 delays initiation of chemically induced hepatocarcinogenesis. Mol Carcinog 45: 934-942, 2006.
5. INAGAKI T. et al.: Endocrine regulation of the fasting response by PPARa-mediated induction of fibroblast growth factor 21. Cell Metab 5: 415-425, 2007.
6. KHARITONENKOV A. et al.: FGF-21 as a novel metabolic regulator. J Clin Invest 115: 1627-1635, 2005.
7. KHARITONENKOV A. et al.: The metabolic state of diabetic monkeys is regulated by fibroblast growth factor-21. Endocrinology 148: 774-781, 2007.
8. LUNDASEN T. et al.: Circulating intestinal fibroblast growth factor 19 has a pronounced diurnal variation and modulates hepatic bile acid synthesis in man. J Intern Med 260: 530-536, 2006.
9. NICHOLES K. et al.: A mouse model of hepatocellular carcinoma: ectopic expression of fibroblast growth factor 19 in skeletal muscle of transgenic mice. Am J Pathol 160: 2295-2307, 2002.
10. NISHIMURA T. et al.: Identification of a novel FGF, FGF-21, preferentially expressed in the liver. Biochim Biophys Acta 1492: 203-206, 2000.
11. ORNITZ D. M. et al.: Fibroblast growth factors. Genome Biol 2: REVIEWS3005, 2001
12. SHIMADA T. et al.: FGF-23 is a potent regulator of vitamin D metabolism and phosphate homeostasis. J Bone Miner Res 19: 429-435, 2004.
13. TOMLINSON E. et al.: Transgenic mice expressing human fibroblast growth factor-19 display increased metabolic rate and decreased adiposity. Endocrinology 143: 1741-1747, 2002.
14. WENTE W. et al.: Fibroblast growth factor-21 improves pancreatic beta-cell function and survival by activation of extracellular signal-regulated kinase 1/2 and Akt signaling pathways. Diabetes 55: 2470-2478, 2006.
15. ANGELIN B. et al.: Circulating fibroblast growth factors as metabolic regulators—a critical appraisal. Cell Metab. 2012 Dec 5; 16(6): 693-705.
16. ZHAO Y. et al.: FGF21 as a therapeutic reagent. Adv Exp Med Biol, 2012; 728: 214-28.

The gut peptide glucagon-like peptide-1 (GLP-1) is an incretin hormone and secreted in a nutrient-dependent manner. It stimulates glucose-dependent insulin secretion. GLP-1 also promotes beta-cell proliferation and controls glycemia via additional actions on glucose sensors, inhibition of gastric emptying, food intake and glucagon secretion. Furthermore, GLP-1 stimulates insulin secretion and reduces blood glucose in human subjects with Type 2 diabetes. Exogenous administration of bioactive GLP-1, GLP-1(7-27) or GLP-1(7-36 amide), in doses elevating plasma concentrations to approximately 3-4 fold physiological postprandial levels fully normalizes fasting hyperglycaemia in Type 2 diabetic patients (Nauck, M. A. et al. (1997) Exp Clin Endocrinol Diabetes, 105, 187-197). The human GLP-1 receptor (GLP-1R) is a 463 amino acid heptahelical G protein-coupled receptor widely expressed in pancreatic islets, kidney, lung, heart and multiple regions of the peripheral and central nervous system. Within islets, the GLP-1R is predominantly localized to islet beta-cells. Activation of GLP-1 R signalling initiates a program of differentiation toward a more endocrine-like phenotype, in particular the differentiation of progenitors derived from human islets into functioning beta-cells (Drucker, D. J. (2006) Cell Metabolism, 3, 153-165).

Unfortunately, each of FGF-21 and bioactive GLP-1, as well as other known drugs have limited efficacy by themselves to the complex and multifactorial metabolic dysfunctions which can be observed in Type 2 diabetes or other metabolic disorders. This applies also for the efficacy in lowering the blood glucose levels by said compounds themselves.

According to the present invention it has surprisingly been found that FGF-21 fusion proteins comprising an FGF-21 agonist fused to a GLP-1 R agonist significantly lowered blood glucose levels in a synergistic manner up to normo-glycaemic levels.

### TECHNICAL PROBLEMS UNDERLYING PRESENT INVENTION

Present invention is based on in vitro and animal studies of the inventors using fusion proteins comprising a FGF-21 agent fused to a GLP1 R-agonist and using FGF-21 compounds and GLP-1-R agonists.

The inventors surprisingly found that FGF-21 fusion proteins comprising an FGF-21 agonist fused to a GLP-1 R agonist lowered blood glucose levels in a synergistic manner up to normo-glycaemic levels and comparably to the effects achieved by administration of the individual components.

The above overview does not necessarily describe all problems solved by present invention.

### SUMMARY OF THE INVENTION

The Following Aspects are Encompassed by the Present Invention:

In a first aspect, present invention concerns a fusion protein comprising the polypeptide with structure A-B-C or C-B-A or B-A-C or B-C-A or A-C-B or C-A-B or A-B-C-B-C or A-C-B or A-B-C-B or A-C-B-C, wherein

A is a GLP-1 R (glucagon-like peptide-1 receptor) agonist and

C is an FGF-21 (fibroblast growth factor 21) compound and

B is a Linker comprising about 1 to 1000 amino acids or wherein

B is a Linker comprising about 0 to 1000 amino acids.

In a second aspect, present invention concerns the fusion protein of the present invention for use as a medicament.

In a third aspect, the present invention concerns a pharmaceutical composition comprising the fusion protein of the present invention together with a pharmaceutically acceptable excipient.

In a fourth aspect, present invention concerns the fusion protein of the present invention or a pharmaceutical composition comprising the fusion protein of the present invention together with a pharmaceutically acceptable excipient for use as a medicament.

In a fifth aspect, present invention concerns an article of manufacture comprising a) the fusion protein or the pharmaceutical composition of the present invention and b) a container or packaging material.

In a sixth aspect, the present invention concerns a method of treating a disease or disorder of a patient, in which the increase of FGF-21 receptor autophosphorylation or in which the increase of FGF-21 efficacy is beneficial for the curing, prevention or amelioration of the disease or disorder, wherein the method comprises administration to the patient of a fusion protein or the pharmaceutical composition of present invention.

In a seventh aspect, the present invention concerns a method of treating a cardiovascular disease and/or diabetes mellitus and/or at least one metabolic syndrome which increases the risk of developing a cardiovascular disease and/or diabetes mellitus, preferably Type 2-diabetes in a patient comprising the administration to the patient of a fusion protein or the pharmaceutical composition of present invention.

In an eighth aspect, the present invention concerns a method of lowering plasma glucose levels, of lowering the lipid content in the liver, of treating hyperlipidemia, of treating hyperglycemia, of increasing the glucose tolerance, of decreasing insulin tolerance, of increasing the body temperature, and/or of reducing weight of a patient comprising the administration to the patient of a fusion protein or the pharmaceutical composition of present invention.

In a ninth aspect, present invention concerns a nucleic acid encoding the fusion protein of present invention, preferably comprising or consisting of one of the following nucleic acid sequences:

a) a nucleic acid sequence according to one of the sequences with SEQ ID NOs: 27 to 38,

b) a nucleic acid coding for a protein sequence according to SEQ ID NOs: 15 to 26 and 39 to 44,

c) a nucleic acid hybridizing under stringent conditions with a nucleic acid according to a) or b).

In a tenth aspect, the present invention concerns a vector comprising the nucleic acid of present invention suitable for expression of the encoded protein in a eukaryotic or prokaryotic host.

In an eleventh aspect, the present invention concerns a cell stably or transiently carrying the vector of present invention and capable of expressing the fusion protein of present invention under appropriate culture conditions.

In a twelfth aspect, the present invention concerns a method of preparing the fusion protein of present invention comprising

a) cultivating a culture of cells of present invention under appropriate culture conditions for the fusion protein to be expressed in the cell, or

b) harvesting or purifying the fusion protein from a culture comprising cells of present invention that have been cultivated under appropriate conditions for the fusion protein to be expressed, or

c) cultivating the cells of present invention according to step a) and purifying the fusion protein according to step b) and optionally

d) cleaving of the His-tag using a protease if the fusion protein is a fusion protein comprising a His-tag.

General Description

Before the present invention is described in detail below, it is to be understood that this invention is not limited to the particular methodology, protocols and reagents described herein, as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. The same applies to the term “includes” and variations thereof such as “including” and “inclusion”.

Sequences: All sequences referred to herein are disclosed in the attached sequence listing that, with its whole content and disclosure, is a part of this specification. A summary of the sequences disclosed herein is provided below:

FGF-21 compounds

| | |
|---|---|
| SEQ ID NO: 1 | Human FGF-21 - including signal sequence |
| | (Native Human FGF-21 - including signal sequence) |
| SEQ ID NO: 2 | FGF-21 mutein |
| | (G + Native Human FGF-21 - including signal sequence) |
| SEQ ID NO: 3 | FGF-21 H29-S209/Mature FGF-21 |
| | (Native Human FGF-21 without signal sequence) |

GLP1-agonists

| | |
|---|---|
| SEQ ID NO: 4 | Exenatide |
| SEQ ID NO: 5 | Human GLP-1(7-37) |
| SEQ ID NO: 6 | Oxyntomodulin |
| SEQ ID NO: 7 | Human GLP-1(7-36)NH2 |
| SEQ ID NO: 8 | Exendin-4 |
| SEQ ID NO: 10 | Lixisenatide |
| SEQ ID NO: 10 | Lixisenatide |

Functional moieties for constructing the linker

| | |
|---|---|
| SEQ ID NO: 11 | Factor Xa cleavage site |
| SEQ ID NO: 12 | Pasylation unit sequence |
| SEQ ID NO: 13 | Pasylation sequence with site for covalent modification (C) |
| SEQ ID NO: 14 | Protease cleavage site |

Fusion proteins

| | |
|---|---|
| SEQ ID NO: 15 | Exenatide-FactorXa-cleavage site-FGF21 |
| SEQ ID NO: 16 | His-SUMO-Exenatide- FactorXa-cleavage site-FGF21 |
| SEQ ID NO: 17 | Exenatide-FGF21 |
| SEQ ID NO: 18 | His-SUMO-Exenatide-FGF21 |
| SEQ ID NO: 19 | His-SUMO-Exenatide-GGGRR-FGF21 |
| SEQ ID NO: 20 | Exenatide-GGGRR-FGF21 |
| SEQ ID NO: 21 | His-SUMO-Lixisenatide-FGF21 |
| SEQ ID NO: 22 | Lixisenatide-FGF21 |
| SEQ ID NO: 23 | His-SUMO-Lixisenatide- FactorXa- cleavage site -FGF21 |
| SEQ ID NO: 24 | Lixisenatide- FactorXa- cleavage site -FGF21 |
| SEQ ID NO: 25 | His-SUMO-Lixisenatide-GGGRR-FGF21 |
| SEQ ID NO: 26 | Lixisenatide-GGGRR-FGF21 |

Constructs for fusion proteins (DNA sequences)

| | |
|---|---|
| SEQ ID NO: 27 | Construct: CR8829 |
| SEQ ID NO: 28 | Construct: CR8846 |
| SEQ ID NO: 29 | Construct: CR8847 |
| SEQ ID NO: 30 | Construct: CR8848 |
| SEQ ID NO: 31 | Construct: CR8849 |
| SEQ ID NO: 32 | Construct: CR8850 |
| SEQ ID NO: 33 | Construct: CR9443 |
| SEQ ID NO: 34 | Construct: CR9444 |
| SEQ ID NO: 35 | Construct: CR9445 |
| SEQ ID NO: 36 | Construct: CR9446 |
| SEQ ID NO: 37 | Construct: CR9447 |
| SEQ ID NO: 38 | Construct: CR9448 |

Fusion proteins

| | |
|---|---|
| SEQ ID NO: 39 | CR9443 His-SUMO-FGF21-GSGSIEGR- Exenatide |
| | 36698, 08 Da Linker plus intact Factor Xa cleavage site |
| SEQ ID NO: 40 | CR9444 His-SUMO-FGF21-GSGSIEGQ- Exenatide |
| | 36670, 02 Da Linker plus mutated/defect Factor Xa cleavage site |
| SEQ ID NO: 41 | CR9445 His-SUMO -Exenatide-IEGQ- FGF21 |
| | 36381, 76 Da Mutated/defect Factor Xa cleavage site as linker |
| SEQ ID NO: 42 | CR9446 His-SUMO- Exenatide -APASPAS-FGF21 |
| | 36535, 93 Da Linker based on PAS sequence |
| SEQ ID NO: 43 | CR9447 His-SUMO- Exenatide -APASCPAS- FGF21 |
| | 36638, 07 Da Linker based on PAS sequence plus Cystein for |
| | potential modification |
| SEQ ID NO: 44 | CR9448 His-SUMO -Exenatide-GSGS- FGF21 |
| | 36242, 57 Da GSGS-linker |
| SEQ ID NO: 45 | FGF21-GSGSIEGR-Exenatide |
| | 24306, 16 Da (GSGSIEGR = linker) |
| SEQ ID NO: 46 | FGF21-GSGSIEGQ-Exenatide |
| | 24278, 10 Da (GSGSIEGQ = linker) |
| SEQ ID NO: 47 | Exenatide-IEGQ-FGF21 |
| | 23989, 84 Da (IEGQ = linker) |
| SEQ ID NO: 48 | Exenatide-APASPAS-FGF21 |
| | 24144, 01 Da (APSPAS = linker) |
| SEQ ID NO: 49 | Exenatide-APASCPAS-FGF21 |
| | 24246 ,14 Da (APSCPAS = linker) |
| SEQ ID NO: 50 | Exenatide-GSGS-FGF21 |
| | 23850, 64 Da (GSGS = linker) |
| SEQ ID NO: 51 | Exenatide-GG-ABD-GG-FGF21 |
| | 28820, 40 Da (GG-ABD-GG = linker) |
| SEQ ID NO: 52 | Exenatide-GGGGS-ABD-GGGGS-FGF21 |
| | 29222, 76 Da (GGGGS-ABD-GGGGS = linker) |
| SEQ ID NO: 53 | Exenatide-FGF21-GG-ABD |
| | 28706, 29 Da (GG-ABD = linker) |
| SEQ ID NO: 54 | Exenatide-FGF21-GGGGS-ABD |
| | 28907, 48 Da (GGGGS-ABD = linker) |
| SEQ ID NO: 55 | Exenatide-FGF21-GG-ABD-GG-FGF21 |
| | 48195, 17 Da (GG-ABD-GG = linker) |
| SEQ ID NO: 56 | Exenatide-FGF21-GGGGS-ABD-GGGGS-FGF21 |
| | 48597, 54 Da (GGGGS-ABD-GGGGS = linker) |
| SEQ ID NO: 57 | Exenatide- GGGGS-His-GGGGS -FGF21 |
| | 25134, 92 Da (GGGGS-His-GGGGS = linker) |
| SEQ ID NO: 58 | Exenatide-GGGGS-His-GGGGS-ABD-GG-FGF21 |
| | 30278, 83 Da (GGGGS-His-GGGGS-ABD-GG = linker) |
| SEQ ID NO: 59 | Exenatide-(B)0-1000-FGF21 mutein-Cys |
| | (B = linker) |
| SEQ ID NO: 60 | Exenatide-(B)0-1000-FGF21 mutein-Lys |
| | (B = linker) |
| SEQ ID NO: 61 | Exenatide-GG-Cys-(G)21-FGF21 |
| | 25009, 73 Da (GG-Cys-(G)21 = linker) |
| SEQ ID NO: 62 | Exenatide-GG-Lys-(G)21-FGF21 |
| | 25035, 78 Da (GG-Lys-(G)21 = linker) |
| SEQ ID NO: 63 | Exenatide-IgG 1 Asp103-Lys329-FGF21 |
| | 49314, 49 Da (GG-IgG 1 Asp103-Lys329-GG = linker) |
| SEQ ID NO: 64 | Exenatide-IgG1 Pro120-Lys329-FGF21 |
| | 47598, 53 Da (GG-IgG1 Pro120-Lys329-GG = linker) |
| SEQ ID NO: 65 | Exenatide-IgG1 Pro120-Lys329 mutated-FGF21 |
| | 47572, 41 Da (GG-IgG1 Pro120-Lys329 mutated-GG = linker) |
| SEQ ID NO: 66 | Exenatide- IgG1 Pro120-Lys222-FGF21 |
| | 35541, 10 Da (GG-IgG1 Pro120-Lys222-GG linker) |

Constructs for fusion proteins (DNA sequences)

| | |
|---|---|
| SEQ ID NO: 67 | Exenatide-GGGGS-ABD-GGGGS-FGF21 |
| SEQ ID NO: 68 | Exenatide-FGF21-GGGGS-ABD |
| SEQ ID NO: 69 | Exenatide-FGF21-GGGGS-ABD-GGGGS-FGF21 |
| SEQ ID NO: 70 | Exenatide-GG-ABD-GG-FGF21 |
| | (GG-ABD-GG = linker) |
| SEQ ID NO: 71 | Exenatide-FGF21-GG-ABD |
| | (GG-ABD = linker) |
| SEQ ID NO: 72 | Exenatide-FGF21-GG-ABD-GG-FGF21 |
| | (GG-ABD-GG = linker) |
| SEQ ID NO: 73 | Exenatide-GGGGS-His-GGGGS-FGF21 |
| | (GGGGS-His-GGGGS = linker) |
| SEQ ID NO: 74 | Exenatide-GGGGS-His-GGGGS-ABD-GG-FGF21 |
| | (GGGGS-His-GGGGS-ABD-GG = linker) |
| SEQ ID NO: 75 | Exenatide-GG-Cys-(G)21-FGF21 |
| | (GG-Cys-(G)21 = linker) |
| SEQ ID NO: 76 | Exenatide-GG-Lys-(G)21-FGF21 |
| | (GG-Lys-(G)21 = linker) |
| SEQ ID NO: 77 | Exenatide-GG-IgG 1 Asp103-Lys329-GG-FGF21 |
| | (GG-IgG 1 Asp103-Lys329-GG = linker) |
| SEQ ID NO: 78 | Exenatide-GG-IgG1 Pro120-Lys329-GG-FGF21 |
| | (GG-IgG1 Pro120-Lys329-GG = linker) |

Functional moieties for constructing the linker

| | |
|---|---|
| SEQ ID NO: 79 | Fc fragment 1: IgG 1 Asp103-Lys329 |
| SEQ ID NO: 80 | Fc fragment 2: IgG1 Pro120-Lys329 |
| SEQ ID NO: 81 | Fc fragment 3: IgG1 Pro120-Lys329 mutated |
| SEQ ID NO: 82 | Fc fragment 4: IgG1 Pro120-Lys222 |
| SEQ ID NO: 83 | GG-(IgG 1 Asp103-Lys329)-GG |
| SEQ ID NO: 84 | GG-(IgG1 Pro120-Lys329)-GG |
| SEQ ID NO: 85 | GG-(IgG1 Pro120-Lys329 mutated)-GG |
| SEQ ID NO: 86 | GG-(IgG1 Pro120-Lys222)-GG |
| SEQ ID NO: 87 | Albumin-Binding Domain (ABD) |
| SEQ ID NO: 88 | GG-Albumin-Binding Domain-GG |
| | (GG-ABD-GG = linker) |
| SEQ ID NO: 89 | GGGGS-Albumin-Binding Domain-GGGGS |
| | (GGGGS-ABD-GGGGS = linker) |
| SEQ ID NO: 90 | Human Serum Albumine (HSA) |
| SEQ ID NO: 91 | Human Serum Albumine (HSA) with linker |
| | (GG[GGGGS]3)A-HSA-GG[GGGGS]3)A) |
| SEQ ID NO: 92 | Sequence with multiple His-residues 1 |
| SEQ ID NO: 93 | Sequence with multiple His-residues 1 |
| SEQ ID NO: 94 | FGF21 (without signal sequence) based linker |
| SEQ ID NO: 95 | PASylation Sequence 1 |
| SEQ ID NO: 96 | PASylation Sequence 2 |
| SEQ ID NO: 97 | PASylation Sequence 3 |
| SEQ ID NO: 98 | PASylation Sequence 4 |
| SEQ ID NO: 99 | PASylation Sequence 5 |
| SEQ ID NO: 100 | PASylation Sequence 6 |
| SEQ ID NO: 101 | PASylation Sequence 7 |

GLP1-agonists

| | |
|---|---|
| SEQ ID NO: 102 | FGF-21 mutein |
| | (G + FGF-21 without signal sequence) |

Constructs for fusion proteins (DNA sequences)

| | |
|---|---|
| SEQ ID NO: 103 | Exenatide-GG-IgG1 Pro120-Lys329 mutated-GG-FGF21 |
| | (GG-IgG1 Pro120-Lys329 mutated-GG = linker) |
| SEQ ID NO: 104 | Exenatide-GG-IgG1 Pro120-Lys222-GG-FGF21 |
| | (GG-IgG1 Pro120-Lys222-GG = linker) |

The term “about” when used in connection with a numerical value is meant to encompass numerical values within a range having a lower limit that is 5% smaller than the indicated numerical value and having an upper limit that is 5% larger than the indicated numerical value

Definitions

The term “pharmaceutical composition” as used herein includes (but is not limited to) the formulation of the active compound with a carrier. In one embodiment, the formulation comprises the fusion protein as described herein and particularly the fusion protein of the first aspect of present invention. The carrier can e.g. be an encapsulating material providing a capsule in which the active component(s)/ingredient(s) with or without other carriers, is surrounded by a carrier, which is thus, in association with it. The carrier can also be suitable for a liquid formulation of the active ingredient(s), and preferably be itself a liquid. The carrier can also be any other carrier as suitable for the intended formulation of the pharmaceutical composition.

“Pharmaceutically acceptable” means approved by a regulatory agency of the Federal or a state government or a supra-national organisation of states such as the European Union or an economic area such as the European Economic Area or listed in the U.S. Pharmacopeia or other generally recognized pharmacopeia in a given country or economic area for use in animals, and more particularly in humans.

The term “carrier”, as used herein, refers to a pharmacologically inactive substance such as but not limited to a diluent, excipient, or vehicle with which the therapeutically active ingredient is administered. Such pharmaceutical carriers can be liquid or solid. Liquid carrier include but are not limited to sterile liquids, such as saline solutions in water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. In the context of the pharmaceutical composition comprising the herein-described fusion proteins and particularly the fusion proteins according to the first or third aspect, a sterile solution for injection or a dry-powder formulation for dissolution are among the preferred formulations

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Examples of suitable pharmaceutical carriers are described in “Remington's Pharmaceutical Sciences” by E. W. Martin. The term “active material” refers to any material with therapeutic activity, such as one or more active ingredients. The active ingredients to be employed as therapeutic agents can be easily prepared in such unit dosage form with the employment of pharmaceutical materials which themselves are available in the art and can be prepared by established procedures.

The term “active ingredient” refers to the substance in a pharmaceutical composition or formulation that is biologically active, i.e. that provides pharmaceutical value. A pharmaceutical composition may comprise one or more active ingredients which may act in conjunction with or independently of each other.

The active ingredient can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as but not limited to those derived from sodium, potassium, ammonium, calcium, ferric hydroxides, isopropylamine, triethylarnine, 2-ethylamino ethanol, histidine, procaine, and the like.

As used herein, “unit dosage form” refers to physically discrete units suitable as unitary dosages for human and/or animal subjects, each unit containing a predetermined quantity of active material (e.g., about 50 to about 500 mg of fusion protein and optionally comprising a pharmaceutically effective amount of DPP IV inhibitor and/or of anti-diabetic drug) calculated to produce the desired therapeutic effect in association with the required pharmaceutical diluent, carrier or vehicle. The specifications for the unit dosage forms herein described are dictated by and are directly dependent on (a) the unique characteristics of the active material and the particular therapeutic effect to be achieved, and (b) the limitation inherent in the art of compounding such an active material for therapeutic use in animals or humans, as disclosed in this specification, these being features of the present invention. Examples of suitable unit dosage forms in accord with this invention are vials, tablets, capsules, troches, suppositories, powder packets, wafers, cachets, ampules, pre-filled syringes, segregated multiples of any or a mixture of the foregoing, and other forms as herein described or generally known in the art. One or more such unit dosage forms comprising the fusion protein can be comprised in an article of manufacture of present invention, optionally further comprising one or more unit dosage forms of an anti-diabetic drug (e.g. a blister of tablets comprising as active ingredient the anti-diabetic drug) or comprising one or more unit dosage forms of a DPP IV-inhibitor (e.g. a blister of tablets comprising as active ingredient a DPP IV-inhibitor) or both (i.e. the fusion protein, the anti-diabetic drug and the DPP IV inhibitor).

The following preparations are illustrative of the preparation of the unit dosage forms of the present invention, and not as a limitation thereof. Several dosage forms may be prepared embodying the present invention. For example, a unit dosage per vial may contain 0,5 ml, 1 ml, 2 ml, 3 ml, 4 ml, 5 ml, 6 ml, 7 ml, 8 ml, 9 ml, 10 ml, 15 ml, or 20 ml of fusion protein comprising a therapeutically effective amount of fusion protein ranging from about 40 to about 500 mg of fusion protein and preferably range from about 0,5 to 1 ml comprising a therapeutically effective amount such as about 40 to about 500 mg of the fusion protein. If necessary, these preparations can be adjusted to a desired concentration by adding a sterile diluent to each vial. In one embodiment, the ingredients of formulation of the invention are supplied either separately or mixed together in unit dosage form, for example, as a dry lyophilized powder or water free concentrate in a hermetically sealed container such as a vial, an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The formulations as herein described include bulk drug compositions useful in the manufacture of pharmaceutical compositions (e.g., compositions that are suitable for administration to a subject or patient) which can be used in the preparation of unit dosage forms. In a preferred embodiment, a composition of the invention is a pharmaceutical composition. Such compositions comprise a prophylactically or therapeutically effective amount of one or more prophylactic or therapeutic agents (e.g., a fusion protein of the invention, a DPP-IV inhibitor, an anti-diabetic drug or another prophylactic or therapeutic agent), and a pharmaceutically acceptable carrier. Preferably, the pharmaceutical compositions are formulated to be suitable for the route of administration to a subject.

The active materials, agents or ingredients (e.g. the fusion proteins, anti-diabetic drugs or DPP IV-inhibitors) can be formulated as various dosage forms including solid dosage forms for oral administration such as capsules, tablets, pills, powders and granules, liquid dosage forms for oral administration such as pharmaceutically acceptable emulsions, microemulsions, solutions, suspensions, syrups and elixirs, injectable preparations, for example, sterile injectable aqueous or oleaginous suspensions, compositions for rectal or vaginal administration, preferably suppositories, and dosage forms for topical or transdermal administration such as ointments, pastes, creams, lotions, gels, powders, solutions, sprays, inhalants or patches.

In a specific embodiment, the term “pharmaceutically acceptable” means approved by a regulatory agency of the U.S. Federal or a state government or the EMA (European Medicines Agency) or listed in the U.S. Pharmacopeia Pharmacopeia (United States Pharmacopeia-33/National Formulary-28 Reissue, published by the United States Pharmacopeial Convention, Inc., Rockville Md., publication date: April 2010) or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. For the use of (further) excipients and their use see also “Handbook of Pharmaceutical Excipients”, fifth edition, R. C. Rowe, P. J. Seskey and S. C. Owen, Pharmaceutical Press, London, Chicago. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the antibody, preferably in purified form, together with a suitable amount of carrier so as to provide the form for proper administration to the patient. The formulation should suit the mode of administration.

Generally, the ingredients of compositions of the invention are supplied either separately or mixed together in a unit dosage form, for example, as a dry formulation for dissolution such as a lyophilized powder, freeze-dried powder or water free concentrate in a hermetically sealed container, such as an ampoule or sachette indicating the quantity of active agent. The ingredients of compositions of the invention can also be supplied as admixed liquid formulation (i.e. injection or infusion solution) in a hermetically sealed container such as an ampoule, sachette, a pre-filled syringe or autoinjector, or a cartridge for a reusable syringe or applicator (e.g. pen or autoinjector). Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides that the formulation is packaged in a hermetically sealed container such as an ampoule or sachette indicating the quantity of antibody. In one embodiment, the formulation of the invention comprising an antibody is supplied as a dry formulation, such as a sterilized lyophilized powder, freeze-dried powder, spray-dried powder or water free concentrate in a hermetically sealed container and can be reconstituted, e.g., with water or saline to the appropriate concentration for administration to a subject. In another embodiment the antibody or antigen binding fragment thereof is supplied as a liquid formulation such as an injection or infusion solution. In one embodiment, the formulation of the invention comprising an antibody is supplied as a dry formulation or as a liquid formulation in a hermetically sealed container at a unit dosage of at least 40 mg, at least 50 mg, at least 75 mg, at least 100 mg, at least 150 mg, at least 200 mg, at least 250 mg, at least 300 mg, at least 350 mg, at least 400 mg, at least 450 mg, or at least 500 mg, of fusion protein. The lyophilized formulation of the invention comprising an antibody should be stored at between 2 and 8° C. in its original container and the antibody should be administered within 12 hours, preferably within 6 hours, within 5 hours, within 3 hours, or within 1 hour after being reconstituted. The formulation of the invention comprising the fusion protein can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Specific populations treatable by the therapeutic methods and medical uses of the invention include subjects with one or more of the following conditions: subjects with elevated blood glucose levels, subjects with hyperglycemia, subjects with obesity, subjects with diabetes, subjects with type 1 or 2 diabetes, subjects with impaired glucose metabolism, subjects with lowered glucose tolerance, subjects with hyperlipidemia, subjects with diabetes mellitus, subjects with insulin resistance, subjects with hypertension, subjects with hypercholesterolemia, and subjects with cardiovascular disease such as coronary heart disease.

Specific indications treatable by the therapeutic methods and medical uses of the invention include subjects with one or more of the following conditions: subjects with elevated blood glucose levels, subjects with hyperglycemia, subjects with obesity, subjects with diabetes, subjects with type 1 or 2 diabetes, subjects with impaired glucose metabolism, subjects with lowered glucose tolerance, subjects with hyperlipidemia, subjects with diabetes mellitus, subjects with insulin resistance, subjects with hypertension, subjects with hypercholesterolemia, and subjects with cardiovascular disease such as coronary heart disease.

The conditions or disorders as listed for the above populations or subjects are conditions or disorders, for which treatment with the fusion protein of the invention is especially suitable.

However, depending on the severity of the afore-mentioned diseases and conditions, the treatment of subjects with the fusion proteins of the invention may be contraindicated for certain diseases and conditions.

The term “adverse effect” (or side-effect) refers to a harmful and undesired effect resulting from a medication. An adverse effect may be termed a “side effect”, when judged to be secondary to a main or therapeutic effect. Some adverse effects occur only when starting, increasing or discontinuing a treatment. Adverse effects may cause medical complications of a disease and negatively affect its prognosis. Examples of side effects are allergic reactions, vomiting, headache, or dizziness or any other effect herein described.

The terms “elevated blood glucose levels”, “elevated blood sugar”, “hyperglycemia”, “hyperglycaemia” and “high blood sugar” are used synonymously herein and refer to a condition in which an excessive amount of glucose , e.g. a glucose level of 200 mg/dL or more, circulates in the blood plasma. Reference ranges for blood tests are 11.1 mmol/l, but symptoms may not start to become noticeable until even higher values such as 250-300 mg/dl or 15-20 mmol/l. According to the American Diabetes Association guidelines, a subject with a consistent range between 100 and 126 mg/dL is considered hyperglycemic, while above 126 mg/dl or 7 mmol/l is generally held to have Diabetes. Chronic levels exceeding 7 mmol/l (125 mg/dl) can produce organ damage.

As used herein, a “patient” means any mammal, reptile or bird that may benefit from a treatment with a pharmaceutical composition as described herein. Preferably, a “patient” is selected from the group consisting of laboratory animals (e.g. monkey, mouse or rat), domestic animals (including e.g. guinea pig, rabbit, horse, donkey, cow, sheep, goat, pig, chicken, camel, cat, dog, turtle, tortoise, snake, or lizard), or primates including chimpanzees, bonobos, gorillas and human beings. It is particularly preferred that the “patient” is a human being.

The terms “subject” or “individual” are used interchangeably herein. As used herein, a “subject” refers to a human or a non-human animal (e.g. a mammal, avian, reptile, fish, amphibian or invertebrate; preferably an individual that can either benefit from one of the different aspects of present invention (e.g. a method of treatment or a drug identified by present methods) or that can be used as laboratory animal for the identification or characterisation of a drug or a method of treatment. The subject can e.g. be a human, a wild-animal, domestic animal or laboratory animal; examples comprise: mammal, e.g. human, non-human primate (chimpanzee, bonobo, gorilla), dog, cat, rodent (e.g. mouse, guinea pig, rat, hamster or rabbit, horse, donkey, cow, sheep, goat, pig, camel; avian, such as duck, dove, turkey, goose or chick; reptile such as: turtle, tortoise, snake, lizard, amphibian such as frog (e.g. Xenopus laevis); fish such as koy or zebrafish; invertebrate such as a worm (e.g. C. elegans) or an insect (such as a fly, e.g. Drosophila melanogaster). The term subject also comprises the different morphological developmental stages of avian, fish, reptile or insects, such as egg, pupa, larva or imago. The term “subject” comprises the term “patient”. According to a preferred embodiment, the subject is a “patient”.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease or disorder means preventing that a disorder occurs in subject. As used herein, the expressions “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity.

As used herein, “administering” includes in vivo administration, as well as administration directly to tissue ex vivo, such as vein grafts.

An “effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

The term “Fibroblast Growth Factor 21” or FGF-21 or FGF21 refers to any FGF-21 as known in the art and particularly refers to human FGF-21 and more particularly refers to FGF-21 according to any of the sequences herein described.

A “FGF-21 compound” as used herein is a compound having FGF-21 activity, in particular comprising (i) native FGF-21 or (ii) a FGF-21 mimetic with FGF-21 activity or (iii) an FGF-21 fragment with FGF-21 activity.

The term “native FGF-21” as used herein refers to the naturally occurring FGF-21 or a variant being substantially homologous to native FGF-21. Typically, such FGF-21 variant is biologically equivalent to native FGF-21, i.e. is capable of exhibiting all or some properties in an identical or similar manner as naturally occurring FGF-21. In preferred embodiments the native FGF-21 is mammalian FGF-21, preferably selected from the group consisting of mouse, rat, rabbit, sheep, cow, dog, cat, horse, pig, monkey, and human FGF-21. The FGF-21 mutein as shown in SEQ ID NO: 102 is particularly preferred. Native human FGF-21 comprises a signal sequence (see SEQ ID NO: 1). FGF-21 compounds without signal sequence, as shown in SEQ ID NO: 3, are particularly preferred.

A variant being “substantially homologous” to native FGF-21 is characterized by a certain degree of sequence identity to FGF-21 from which it is derived. More precisely, in the context of the present invention a variant being substantially homologous to FGF-21 exhibits at least 80% sequence identity to FGF-21 and particularly at least 80% sequence identity to FGF-21 according to SEQ ID NO:3.

The term “at least 80% sequence identity” is used throughout the specification with regard to polypeptide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide. FGF-21 variants may additionally or alternatively comprise deletions of amino acids, which may be N-terminal truncations, C-terminal truncations or internal deletions or any combination of these. Such variants comprising N-terminal truncations, C-terminal truncations and/or internal deletions are referred to as “deletion variant” or “fragments” in the context of the present application. The terms “deletion variant” and “fragment” are used interchangeably herein. A fragment may be naturally occurring (e.g. splice variants) or it may be constructed artificially, preferably by gene-technological means. Preferably, a fragment (or deletion variant) has a deletion of up to 1, 2, 3, 4, 5, 6, 7, 8, 9, 10, 15, 20, 25, 30, 35, 40, 45, 50, 55, 60, 65, 70, 75, 80, 85, 90, 95, or 100 amino acids at its N-terminus and/or at its C-terminus and/or internally as compared to the parent polypeptide, preferably at its N-terminus, at its N- and C-terminus, or at its C-terminus. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by the SEQ ID, if not specifically indicated otherwise. For example, a peptide sequence consisting of 105 amino acids compared to the amino acid sequence of FGF-21 according to SEQ ID NO: 1 may exhibit a maximum sequence identity percentage of 50.24% (105/209) while a sequence with a length of 181 amino acids may exhibit a maximum sequence identity percentage of 86.6% (181/209). For example, a peptide sequence consisting of 105 amino acids compared to the amino acid sequence of FGF-21 according to SEQ ID NO: 3 may exhibit a maximum sequence identity percentage of 58.01% (105/181).

The similarity of amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer dot wustl dot edu/) or with the CLUSTAL algorithm (Thompson, J. D., Higgins, D. G. & Gibson, T. J. (1994) Nucleic Acids Res. 22, 4673-80) available e.g. on http://www dot ebi dot ac dot uk/Tools/clustalw/ or on http://www dot ebi dot ac dot uk/Tools/clustalw2/index dot html or on http://npsa-pbil dot ibcp dot fr/cgi-bin/npsa_automat dot pl?page=/NPSA/npsa_clustalw dot html. Preferred parameters used are the default parameters as they are set on http://www dot ebi dot ac dot uk/Tools/clustalw/ or http://www dot ebi dot ac dot uk/Tools/clustalw2/index dot html. The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215: 403-410. BLAST polynucleotide searches are performed with the BLASTN program, score=100, word length=12, to obtain polynucleotide sequences that are homologous to those nucleic acids which encode F, N, or M2-1. BLAST protein searches are performed with the BLASTP program, score=50, word length=3, to obtain amino acid sequences homologous to the F polypeptide, N polypeptide, or M2-1 polypeptide. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:I54-I62) or Markov random fields. When percentages of sequence identity are referred to in the present application, these percentages are calculated in relation to the full length of the longer sequence, if not specifically indicated otherwise.

FGF-21 mimetics with FGF-21 activity comprise FGF-21 molecules carrying alterations to the amino acid chain of native FGF-21 such that they exhibit FGF-21 activity and further exhibit additional properties such as but not limited to modified chemical properties and/or a prolonged serum half-life. FGF-21 mimetics include but are not limited to FGF-21 muteins, FGF-21 fusion proteins and FGF-21 conjugates. A preferred FGF-21 mutein is e.g. the FGF-21 according to SEQ ID NO: 2 and the FGF-21 according to SEQ ID NO: 102.

The term “FGF-21 activity” refers to any known biological activity of naturally occuring FGF-21, such as but not limited to those listed above and in the following:

1) The stimulation of glucose uptake (e.g. in adipocytes such as human or mouse adipocytes, e.g. mouse 3T3-L1 adipocytes) in the presence of insulin and absence of insulin.

2) The increase in glucose-induced insulin secretion from diabetic islets (e.g. from diabetic patients or diabetic test animals such as diabetic rodents or from isolated beta cells from diabetic test animals such as diabetic rodents or isolated islets from diabetic test animals such as diabetic rodents).

3) The decrease of fed and fasting blood glucose levels (e.g. in ob/ob mice, in db/db mice or in 8 week old ZDF rats in a dose-dependent manner).

4) The decrease of fed and fasting triglycerides (e.g. in ob/ob mice, in db/db mice or in 8 week old ZDF rats in a dose-dependent manner).

5) The decrease of fed and fasting glucagon levels (e.g. in ob/ob mice, in db/db mice or in 8 week old ZDF rats in a dose-dependent manner).

6) A lowering of LDL lipoprotein cholesterol and/or raising of HDL lipoprotein cholesterol.

7) An increase in Glut-1 protein or mRNA steady state level.

8) The interaction with other proteins, such as FGF-receptor, especially FGF-receptor 1, 2 or 3 or a part thereof able to interact with FGF-21.

9) The activation of certain signaling pathways, e.g. activation of extracellular signal-related kinase 1/2, activation of the Akt signaling pathway.

The term “FGF-21 activity” also refers to the combination of two or more of any of the above-listed activities and also to a combination of one or more of them with any other known beneficial activity of FGF-21.

“FGF-21 activity” can for example be measured in a FGF-21 activity assay generally known to a person skilled in the art. An FGF-21 activity assay is e.g. a “glucose uptake assay” as described in Kharitonenkov, A. et al. (2005), 115; 1627, No. 6. As an example for the glucose uptake assay, adipocytes are starved for 3 hours in DMEM/0.1% BSA, stimulated with FGF-21 for 24 hours, and washed twice with KRP buffer (15 mM HEPES, pH 7.4, 118 mM NaCl, 4.8 mM KCl, 1.2 mM MgSO₄, 1.3 mM CaCl₂, 1.2 mM KH₂PO₄, 0.1% BSA), and 100 μl of KRP buffer containing 2-deoxy-D-[¹⁴C]glucose (2-DOG) (0.1 μCi, 100 μM) is added to each well. Control wells contains 100 μl of KRP buffer with 2-DOG (0.1 μCi, 10 mM) to monitor for nonspecificity. The uptake reaction is carried out for 1 hour at 37° C., terminated by addition of cytochalasin B (20 μM), and measured using Wallac 1450 MicroBeta counter (PerkinElmer, USA).

Examples of FGF-21 mimetics are

(a) proteins having at least about 96%, in particular 99% amino acid sequence identity to the amino acid sequence shown in SEQ ID NO: 3 and having FGF-21 activity,

(b) FGF-21 fusion proteins comprising native FGF-21, e.g. according to SEQ ID NO:1, or FGF-21 without signal sequence, according to SEQ ID NO: 3, or a functional fragment thereof, or comprising an FGF-21 mutein fused to another polypeptide (e.g. an FGF-21-Fc fusion, GLP-1R agonist fusion protein, an FGF-21-HSA fusion protein)

(c) FGF-21 conjugates, e.g. PEGylated FGF-21, HESylated FGF-21, FGF-21 coupled to a small molecule unit, etc.

Examples of FGF-21 fusion proteins are described in e.g. WO2004/110472 or WO2005/113606, for example a FGF-21-Fc fusion protein or a FGF-21-HSA fusion protein. “Fc” means the Fc portion of an immunoglobulin, e.g. the Fc portion of IgG4. “HSA” means human serum albumin. Such FGF-21 fusion proteins typically show an extended time of action such as but not limited to an extended serum half-life, compared to native FGF-21 or a substantially homologous variant thereof.

The term “conjugate” or “conjugates” as used herein refers to the amino acid chain of native FGF-21 or substantially homologous variants of FGF-21 or to a FGF-21 compound according to SEQ ID NO: 3 that comprise one or more alterations of the amino acid chain allowing for chemical conjugations of the amino acid chain such as but not limited to PEGylation, HESylation, or Polysialylation. Such FGF-21 conjugates typically show an extended time of action such as but not limited to an extended serum half-life, compared to native FGF-21 or a substantially homologous variant thereof.

Examples of FGF-21 conjugates are described in e.g. WO2005/091944, WO2006/050247 or WO2009/089396, for example glycol-linked FGF-21 compounds. Such glycol-linked FGF-21 compounds usually carry a polyethylene glycol (PEG), e.g. at a cysteine or lysine amino acid residue or at an introduced N-linked or O-linked glycosylation site, (herein referred to as “PEGylated FGF-21”). Such PEGylated FGF-21 compounds generally show an extended time of action compared to human FGF-21. Suitable PEGs have a molecular weight of about 20,000 to 40,000 daltons.

“Muteins” typically comprise alterations such as but not limited to amino acid exchanges, additions and/or deletions to the FGF-21 amino acid chain which maintain the FGF-21 activity and typically alter the chemical properties of the amino acid chain, such as but not limited to an increased or decreased glycosylation or amination of the amino acid chain, and/or an increased or decreased potential to be proteolytically degraded and/or an alteration to the electrostatic surface potential of the protein.

Examples of FGF-21 muteins are described in e.g. WO2005/061712, WO2006/028595, WO2006/028714, WO2006/065582 or WO2008/121563. Exemplary muteins are muteins which have a reduced capacity for O-glycosylation when e.g. expressed in yeast compared to wild-type human FGF-21, e.g. human FGF-21 with a substitution at position 167 (serine), e.g. human FGF-21 with one of the following substitutions: Ser167Ala, Ser167Glu, Ser167Asp, Ser167Asn, Ser167Gln, Ser167Gly, Ser167Val, Ser167His, Ser167Lys or Ser167Tyr. Another example is a mutein which shows reduced deamidation compared to wild-type human FGF-21, e.g. a mutein with a substitution at position 121 (asparagine) of human FGF-21, e.g. Asn121Ala, Asn121Val, Asn121Ser, Asn121Asp or Asn121Glu. An alternative mutein is human FGF-21 having one or more non-naturally encoded amino acids, e.g. as described by the general formula in claim 29 of WO2008/121563. Other muteins comprise a substitution of a charged (e.g. aspartate, glutamate) or polar but uncharged amino acids (e.g. serine, threonine, asparagine, glutamine) for e.g. a polar but uncharged or charged amino acid, respectively. Examples are Leu139Glu, Ala145Glu, Leu146Glu, Ile152Glu, Gln156Glu, Ser163Glu, Ile152Glu, Ser163Glu or Gln54Glu. Another mutein is a mutein showing a reduced susceptibility for proteolytic degradation when expressed in e.g. yeast compared to human FGF-21, in particular human FGF-21 with a substitution of Leu153 with an amino acid selected from Gly, Ala, Val, Pro, Phe, Tyr, Trp, Ser, Thr, Asn, Asp, Gln, Glu, Cys or Met. A preferred FGF-21 mutein is the mutated FGF-21 according to SEQ ID NO: 2 (which includes the signal sequence), which contains an additional glycine at the N-terminus. A preferred FGF-21 mutein is the mutated FGF-21 according to SEQ ID NO: 102, which carries a deletion of amino acids 1-28 of human FGF-21 (according to SEQ ID NO: 1) (i.e. which does not contain the signal sequence) and contains an additional glycine at the N-terminus.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331. Examples of groups of amino acids that have side chains with similar chemical properties include

1) aliphatic side chains: glycine, alanine, valine, leucine and isoleucine;

2) aliphatic- hydroxyl side chains: serine and threonine;

3) amide-containing side chains: asparagine and glutamine;

4) aromatic side chains: phenylalanine, tyrosine, and tryptophan;

5) basic side chains: lysine, arginine, and histidine;

6) acidic side chains: aspartate and glutamate, and

7) sulfur-containing side chains: cysteine and methionine.

Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-45. A “moderately conservative” replacement is any change having a nonnegative value in the PAM250 log-likelihood matrix. Given the known genetic code, and recombinant and synthetic DNA techniques, the skilled scientist can readily construct DNAs encoding conservative amino acid variants.

As used herein, “non-conservative substitutions” or “non-conservative amino acid exchanges” are defined as exchanges of an amino acid by another amino acid listed in a different group of the seven standard amino acid groups 1) to 7) shown above.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below.

As applied to polypeptides, the term “substantial similarity” or “substantially similar” means that two peptide sequences, when optimally aligned, such as by the programs GAP or BESTFIT using default gap weights, share at least 80% sequence identity, and preferably at least 90%, 95%, 96%, 98% or 99% or 99.5% sequence identity.

Preferably, residue positions which are not identical differ by conservative amino acid substitutions.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403 410 and (1997) Nucleic Acids Res. 25:3389 402, each of which is herein incorporated by reference.

When percentages of sequence identity are referred to in the present application, these percentages are calculated in relation to the full length of the longer sequence, if not specifically indicated otherwise. This calculation in relation to the full length of the longer sequence applies both to nucleic acid sequences and to polypeptide sequences.

As used herein, the term “fusion protein” refers to Fusion proteins or chimeric proteins created through the joining of two or more protein-encoding nucleic acids which originally coded for separate proteins. Translation of this fusion gene results in a single polypeptide with functional properties derived from each of the original proteins. Recombinant fusion proteins are created artificially by recombinant DNA technology for use in biological research or therapeutics. A recombinant fusion protein is a protein created through genetic engineering of a fusion gene. The present invention relates to recombinant fusion proteins and the terms fusion protein and recombinant fusion protein are used synonymously herein. The fusion proteins described herein comprise typically at least two domains (A and C) and optionally comprise a third component, the linker C that is interspersed between the two domains. The generation of recombinant fusion proteins is known in the art and typically involves removing the stop codon from a cDNA sequence coding for the first protein or polypeptide, then appending the cDNA sequence of the second protein in frame through ligation or overlap extension PCR. That DNA sequence will then be expressed by a cell as a single protein. The protein can be engineered to include the full sequence of both original proteins or polypeptides, or only a portion of either.

The term “linker” as used herein refers to a structural unit that can be inserted in between the two or more other units (e.g. two or more peptides or polypeptides or proteins or a peptide and a protein a polypeptide and a protein, a peptide and a polypeptide) and couple these two or more other units with each other to create one molecule. The coupling of the two units is preferably by covalent bond(s). The term “linker” as used herein also refers to a structural unit that can be attached to the N- or C-terminus of two or more other units (e.g. two or more peptides or polypeptides or proteins or a peptide and a protein a polypeptide and a protein, a peptide and a polypeptide), wherein said two or more other units are directly coupled together. The term “linker” as used herein also refers to combinations of the preceeding definitions, i.e. one structural unit is inserted in between the two or more other units (e.g. two or more peptides or polypeptides or proteins or a peptide and a protein a polypeptide and a protein, a peptide and a polypeptide) and one or more further structural units is / are attached to the N- or C-terminus of two or more other units (e.g. two or more peptides or polypeptides or proteins or a peptide and a protein a polypeptide and a protein, a peptide and a polypeptide). The attachment of the structure unit to the N- or C-terminus of two or more other units is preferably by covalent bond(s).

The structural linker unit can for example comprise

a) one or more polymers (such as a chemical polymer, a protein, polypeptide or peptide, a nucleic acid or derivative thereof (such as a polyamid-nucleic acid), a polycarbon-polymer etc., a polymeric of carbohydrate), wherein the linker can be composed of one polymer or of two or more polymers of the same type or of different types (e.g. linkers composed of two or more peptides are linkers comprising more than one polymer of the same type, whereas e.g. linkers composed of one or more stretches of peptide and nucleic acid such as peptide-nucleic acid-peptide etc. are linkers composed of polymers of different types).

b) a carbohydrate

c) an organic compound-unit

d) a mixture of a and b or a and c or b and c or a and b and c.

Preferred linkers in the context of the present invention are composed of one or more peptides or polypeptides. In one embodiment of the fusion protein of the present invention, the linker is a peptide linker. In one embodiment of the fusion protein of present invention, the linker comprises a functional moiety conferring one or more additional functions beyond that of linking A and C

The linker can be added for improved or independent folding of one or both of the proteins or polypeptides forming the fusion protein and/or for avoiding sterical hindrance and/or for introducing further desired functionalities, e.g. entry sites for covalent attachment of additional moieties, tags for protein purification, protease cleavage sites, protein stabilisation and/or half-life extension of the protein.

Linkers are often composed of flexible residues like glycine and serine so that the adjacent protein domains are free to move relative to one another. Longer linkers are used when it is necessary to ensure that two adjacent domains do not sterically interfere with one another. Examples of the linkers used in the context of present invention are e.g. linkers comprising GS-rich units such as:
a. one or more (GS)ₙ units with n=0, 1, 2, 3, 4, 5, 6, 7, 8, 9, 10, 20, 30, 40, 50, 60, 70, 80, 90, 100;
b. one or more (GGS)ₙ units with n=1, 2, 3, 4, 5, 6, 7, 8, 9, 10, 20, 30, 40, 50, 60, 70, 80, 90, 100;
c. one more (GGSG)ₙ units with n=0, 1, 2, 3, 4, 5, 6, 7, 8, 9, 10, 20, 30, 40, 50, 60, 70, 80, 90, 100;
d. one or more (GₐS_{b})_{c} units with a, b, c=0, 1, 2, 3, 4, 5, 6, 7, 8, 10, 20, 30, 40, 50, 60, 70, 80, 90, 100;
e. one ore more (S_{b}G_{A})_{c} untis with a, b, c=0, 1, 2, 3, 4, 5, 6, 7, 8, 10, 20, 30, 40, 50, 60, 70, 80, 90, 100;

wherein each linker may optionally further contain one more more additional amino acids, preferably selected from the group of histidine, alanine, tryptophane, glutamine, glutamate, aspartate, asparagine, leucine, isoleucine.

Linkers of the present invention comprise between 0, 1 to 1000 amino acids. The linker can also be absent (i.e. 0 amino acids). As stated above, the linkers can be peptides, polypeptides or proteins or can comprise other structural moieties such as stretches of nucleic acid or other polymers. The linker can thus comprise e.g. about 0, 1, 2, 3, 4, 5, 6, 7, 8, 9, 10, 15, 20, 30, 40, 50, 100, 150, 200, 300, 400, 500, 600, 700, 800, 900 or about 1000 amino acids in length.

Typical linker types can e.g. be helical or non-helical, wherein helical linkers are thought to act as rigid spacers separating two domains and non-helical linkers contain proline or are rich in proline, which also leads to structural rigidity and isolation of the linker from the attached domains. This means that both linker types are likely to act as a scaffold to prevent unfavourable interactions between folding domains.

The linker can comprise e.g. one or more of the following functional moieties a) to g):

a) a moiety conferring increased stability and/or half-life to the fusion such as an XTENylation, rPEG or PASylation or HESylation sequence or Elastin-like polypeptides (ELPs);

b) an entry site for covalent modification of the fusion protein such as a cysteine or lysine residue;

c) a moiety with intra- or extracellular targeting function such as a protein-binding scaffold (such as an antibody, antigen-binding fragment, or other proteinaceous non-antibody binding scaffold), a nucleic acid (such as an aptamer, PNA, DNA or the like);

d) a protease cleavage site such as a FactorXa cleavage site or a cleavage site for another (preferably extracellular) protease;

e) an albumin binding domain (ABD);

f) a Fc portion of an immunoglobulin, e.g. the Fc portion of IgG4;

g) an amino acid sequence comprising one or more histidine (His linker, abbreviated as “His”) amino acids, for example HAHGHGHAH.

The linker can consist of the one or more functional moieties, e.g. of a protease cleavage site, a half-life stabilising moiety, an entry site for covalent modification (in its simplest sense a cysteine or lysine) etc. The linker can also comprise one or more amino acids that do not confer additional functionality to the linker and a functionality-conferring moiety. The linker can also comprise or consist of a combination of functional moieties; conceivable examples are e.g.:

A—[stabilizing moiety—protease cleavage site—stabilizing moiety]-C

A—[stabilizing moiety—protease cleavage site—stabilizing moiety]-C

A—[XX//X—protease cleavage site—X//XX]-C

A—[X—entry site for covalent attachment—X//XXXXX]-C

A—[X—protease cleavage site—XX—entry site for covalent attachment—X]-C

Many other combinations of the different moieties are conceivable.

Wherein [] is the linker and X stands for any amino acid and can be=0 to about 1000 amino acids), wherein said listing is non- exhaustive and wherein the arrangement can always also be in the order C-linker-A from N- to C-terminal instead the below listed A- to C- arrangement.

According to some embodiments of the fusion protein of present invention, the linker comprises one or more of the following protease cleavage sites:

a) a factor Xa cleavage site and preferably comprising or consisting of the sequence IEGR (SEQ ID NO:11)

b) a protease cleavage site and preferably comprising or consisting of at least one arginine and more preferably comprising or consisting of the sequence GGGRR (SEQ ID NO: 14).

According to one embodiment of the fusion protein of present invention, the linker comprises or consists of an entry site for covalent modification and preferably comprising or consisting of the sequence according to SEQ ID NO:13, SEQ ID NO: 95, SEQ ID NO: 96, SEQ ID NO: 97, SEQ ID NO: 98, SEQ ID NO: 99, SEQ ID NO: 100, or SEQ ID NO: 101.

According to another embodiment of the fusion protein of present invention, the linker comprises or consists of a protein stabilisation sequence and preferably comprises a PASylation sequence such as the sequence according to SEQ ID NO:12.

According to yet another embodiment of the fusion protein of present invention, the linker comprises or consists of one or more entry sites for covalent modification of the fusion protein such as a cysteine or a lysine and preferably a cysteine.

According to one embodiment of the fusion protein of present invention, B comprises or is IEGR (SEQ ID NO:11), SEQ ID NO:12, SEQ ID NO:13 GGGRR (SEQ ID NO:14), SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, or SEQ ID NO: 101.

The amino acid chain of native FGF-21 or substantially homologous variants of FGF-21 that comprise one or more further amino acid chains. Each amino acid chain is preferably a complete protein, i.e. spanning an entire open reading frame (ORF), or a fragment, domain or epitope thereof. The individual parts of a fusion protein may either be permanently or temporarily connected to each other. Parts of a fusion protein that are permanently connected are translated from a single ORF and are not later separated co- or post-translationally. Parts of fusion proteins that are connected temporarily may also derive from a single ORF but are divided co-translationally due to separation during the translation process or post-translationally due to cleavage of the peptide chain, e.g. by an endopeptidase. Additionally or alternatively, parts of a fusion protein may also be derived from two different ORF and are connected post-translationally, for instance through covalent bonds.

A “GLP-1 R agonist” is defined as a compound which binds to and activates the GLP-1 receptor like GLP-1 (glucagon-like peptide 1). Physiological actions of GLP-1 and/or of the GLP-1R agonist are described e.g. in Nauck, M. A. et al. (1997) Exp. Clin. Endocrinol. Diabetes, 105, 187-195. These physiological actions in normal subjects, in particular humans, include e.g. glucose-dependent stimulation of insulin secretion, suppression of glucagon secretion, stimulation of (pro)insulin biosynthesis, reduction of food intake, deceleration of gastric emptying and/or equivocal insulin sensitivity.

Suitable assays to discover GLP-1 R agonists are described in e.g. Thorkildsen, Chr. et al. (2003), Journal of Pharmacology and Experimental Therapeutics, 307, 490-496; Knudsen, L. B. et al. (2007), PNAS, 104, 937-942, No. 3; Chen, D. et al. (2007), PNAS, 104, 943-948, No. 3; or US2006/0003417 A1 (see e.g. Example 8). In short, in a “receptor binding assay”, a purified membrane fraction of eukaryotic cells harbouring e.g. the human recombinant GLP-1 receptor, e.g. CHO, BHK or HEK293 cells, is incubated with the test compound or compounds in the presence of e.g. human GLP-1, e.g. GLP-1 (7-36) amide which is marked with e.g. ¹²⁵I (e.g. 80 kBq/pmol). Usually different concentrations of the test compound or compounds are used and the IC₅₀ values are determined as the concentrations diminishing the specific binding of human GLP-1. In a “receptor functional assay”, isolated plasma membranes from eukaryotic cells, as e.g. described above, expressing e.g. the human GLP-1 receptor were prepared and incubated with a test compound. The functional assay is carried out by measuring cAMP as a response to stimulation by the test compound. In a “reporter gene assay”, eukaryotic cells, as e.g. described above, expressing e.g. the human GLP-1 receptor and containing e.g. a multiple response element/cAMP response element-driven luciferase reporter plasmid are cultured in the presence of a test compound. cAMP response element-driven luciferase activities are measured as a response to stimulation by the test compound.

Suitable GLP-1R agonists are selected from a bioactive GLP-1, a GLP-1 analog or a GLP-1 substitute, as e.g. described in Drucker, D. J. (2006) Cell Metabolism, 3, 153-165; Thorkildsen, Chr. (2003; supra); Chen, D. et al. (2007; supra); Knudsen, L. B. et al. (2007; supra); Liu, J. et al. (2007) Neurochem Int., 51, 361-369, No. 6-7; Christensen, M. et al. (2009), Drugs, 12, 503-513; Maida, A. et al. (2008) Endocrinology, 149, 5670-5678, No. 11 and US2006/0003417. Exemplary compounds are GLP-1(7-37), GLP-1(7-36)amide, exendin-4, liraglutide, CJC-1131, albugon, albiglutide, exenatide, exenatide-LAR, oxyntomodulin, lixisenatide, geniproside, a short peptide with GLP-1 R agonistic activity and/or a small organic compound with GLP-1 R agonistic activity.

In detail, human GLP-1(7-37) possesses the amino acid sequence of SEQ ID NO: 5. Human GLP-1(7-36)amide possesses the amino acid sequence of SEQ ID NO: 7. Extendin-4 possesses the amino acid sequence of SEQ ID NO: 8. Exenatide possesses the amino acid sequence of SEQ ID NO: 5 and oxyntomodulin the amino acid sequence of SEQ ID NO: 6. The amino acid sequence of lixisenatide is shown in SEQ ID NO: 9. The structure of lixisenatide is based on exendin-4(1-39) modified C-terminally with six additional lysine residues in order to resist immediate physiological degradation by DPP-IV (dipeptidyl peptidase-4). The amino acid sequence of lixisenatide is shown in SEQ ID NO: 10.

The chemical structure of liraglutide is shown in FIG. 4. Liraglutide was obtained by substitution of Lys 34 of GLP-1(7-37) to Arg, and by addition of a C16 fatty acid at position 26 using a γ-glutamic acid spacer. The chemical name is [N-epsilon(gamma-L-glutamoyl(N-alpha-hexadecanoyl)-Lys²⁶, Arg³⁴-GLP-1(7-37)].

The chemical structure of CJC-1131 is shown in FIG. 5. Albumin is attached at the C-terminal of GLP-1 with a d-alanine substitution at position 8. CJC-1131 shows a very good combination of stability and bioactivity.

Other peptides with GLP-1 R agonistic activity are exemplary disclosed in US 2006/0003417, and small organic compounds with GLP-1R agonistic activity are exemplary disclosed in Chen et al. 2007, PNAS, 104, 943-948, No. 3 or Knudsen et al., 2007, PNAS, 104, 937-942.

As used herein, the term “anti-diabetic drug” refers to pharmaceuticals showing a mode of action reducing the symptoms and/or causes of Diabetes and particularly that of Diabetes mellitus. Exemplary anti-diabetic drugs are
a) insulin,
b) thiazolidinedione, e.g. rosiglitazone or pioglitazone (see e.g. WO2005/072769), metformin (N,N-dimethylimidodicarbonimidic-diamide), or
c) sulphonylurea, such as chlorpropamide (4-chloro-N-(propylcarbamoyl)-benzenesulfonamide), tolazamide (N-[(azepan-1-ylamino)carbonyl]-4-methyl-benzenesulfonamide), gliclazide (N-(hexahydrocyclopenta[c]pyrrol-2(1H)-yl-carbamoyl)-4-methylbenzenesulfonamide), or glimepiride (3-ethyl-4-methyl-N-(4-[N-((1r,46-4-methylcyclohexylcarbamoyl)-sulfamoyl]phenethyl)-2-oxo-2,5-dihydro-1 H-pyrrole-1 -carboxamide).

According to the present invention and as used herein “insulin” means naturally occurring insulin, modified insulin or an insulin analogue, including salts thereof, and combinations thereof, e.g. combinations of a modified insulin and an insulin analogue, for example insulins which have amino acid exchanges/deletions/additions as well as further modifications such as acylation or other chemical modification. One example of this type of compound is insulin detemir, i.e. LysB29-tetradecanoyl/des(B30) human insulin. Another example may be insulins in which unnatural amino acids or amino acids which are normally non-coding in eukaryotes, such as D-amino acids, have been incorporated (Geiger, R. et al., Hoppe Seylers Z. Physiol. Chem. (1976) 357, 1267-1270; Geiger, R. et al., Hoppe Seylers Z. Physiol. Chem. (1975) 356, 1635-1649, No. 10; Krail, G. et al., Hoppe Seylers Z. Physiol. Chem. (1971) 352, 1595-1598, No. 11). Yet other examples are insulin analogues in which the C-terminal carboxylic acid of either the A-chain or the B-chain, or both, are replaced by an amide.

“Modified insulin” is preferably selected from acylated insulin with insulin activity, in particular wherein one or more amino acid(s) in the A and/or B chain of insulin is/are acylated, preferably human insulin acylated at position B29 (Tsai, Y. J. et al. (1997) Journal of Pharmaceutical Sciences, 86, 1264-1268, No. 11). Other acetylated insulins are desB30 human insulin or B01 bovine insulin (Tsai, Y. J. et al., supra). Other Examples of acylated insulin are e.g. disclosed in U.S. Pat. No.5,750,497 and U.S. Pat. No. 6,011,007. An overview of the structure-activity relationships for modified insulins, is provided in Mayer, J. P. et al. (2007) Biopolymers, 88, 687-713, No. 5. Modified insulins are typically prepared by chemical and/or enzymatic manipulation of insulin, or a suitable insulin precursor such as preproinsulin, proinsulin or truncated analogues thereof. Further examples of modified insulins include, but are not limited to, the following: (i). ‘Insulin detemir’ differs from human insulin in that the C-terminal threonine in position B30 is removed and a fatty acid residue (myristic acid) is attached to the epsilon-amino function of the lysine in position B29. (ii). ‘Insulin degludec’ differs from human insulin in that the last amino acid is deleted from the B-chain and by the addition of a glutamyl link from LysB29 to a hexadecandioic acid.

An “insulin analogue” is preferably selected from insulin with insulin activity having one or more mutation(s), substitution(s), deletion(s) and/or addition(s), in particular an insulin with a C- and/or N-terminal truncation or extension in the A and/or B chain, preferably des(B30) insulin, PheB1 insulin, B1-4 insulin, AspB28 human insulin (insulin aspart), LysB28/ProB29 human insulin (insulin lispro), LysB03/GluB29 human insulin (insulin glulisine) or GlyA21/ArgB31/ArgB32 human insulin (insulin glargine). The only proviso of an insulin analogue is that it has a sufficient insulin activity. An overview of the structure-activity relationships for insulin analogues, with discussion of which amino acid exchanges, deletions and/or additions are tolerated is provided in Mayer, J. P. et al. (2007; supra). The insulin analogues are preferably such wherein one or more of the naturally occurring amino acid residues, preferably one, two or three of them, have been substituted by another amino acid residue. Further examples of insulin analogues are C-terminal truncated derivatives such as des(B30) human insulin; B-chain N-terminal truncated insulin analogues such as des PheB1 insulin or des B1-4 insulin; insulin analogues wherein the A-chain and/or B-chain have an N-terminal extension, including so-called “pre-insulins” where the B-chain has an N-terminal extension; and insulin analogues wherein the A-chain and/or the B-chain have C-terminal extension. For example one or two Arg may be added to position B1. Examples of insulin analogues are described in the following patents and equivalents thereto: U.S. Pat. No. 5,618,913, EP 0 254 516 A2 and EP 0 280 534 A2. An overview of insulin analogues in clinical use is provided in Mayer J. P. et al. (2007, supra). Insulin analogues or their precursors are typically prepared using gene technology techniques well known to those skilled in the art, typically in bacteria or yeast, with subsequent enzymatic or synthetic manipulation if required. Alternatively, insulin analogues can be prepared chemically (Cao, Q. P. et al. (1986) Biol. Chem. Hoppe Seyler, 367, 135-140, No. 2). Examples of specific insulin analogues are insulin aspart (i.e. AspB28 human insulin); insulin lispro (i.e. LysB28, ProB29 human insulin); insulin glulisine (ie. LysB03, GluB29 human insulin); and insulin glargine (i.e. GlyA21, ArgB31, ArgB32 human insulin).

Exemplary DPP-IV Inhibitors are:

The compound of formula I (FIG. 3), sitagliptin: (R)-4-oxo-4-[3-(trifluoromethyl)-5,6-dihydro[1,2,4]triazolo[4,3-a]-pyrazin-7(8H)-yl]-1-(2,4,5-trifluorophenyl)butan-2-amine, vildagliptin: (S)-1-[N-(3-hydroxy-1-adamantyl)glycyl]pyrrolidine-2-carbonitrile, saxagliptin: (1S,3S,5S)-2-[(2S)-2-amino-2-(3-hydroxy-1-adamantyl)-acetyl]-2-azabicyclo[3.1.0]hexane-3-carbonitrile, linagliptin 8-[(3R)-3-aminopiperidin-1-yl]-7-(but-2-yn-1-yl)-3-methyl-1-[(4-methyl-quinazolin-2-yl)methyl]-3,7-dihydro-1H-purine-2,6-dione) adogliptin (2-({6-[(3R)-3-aminopiperidin-1-yl]-3-methyl-2,4-dioxo-3,4-dihydropyrimidin-1(2H)-yl}methyl)-benzonitrile, and berberine which is a quaternary ammonium salt from the group of isoquinoline alkaloids found in i the roots, rhizomes, stems, and bark of plants such as Berberis, goldenseal (Hydrastis canadensis), and Coptis chinensis.

The pharmaceutical compositions of present application preferably comprise therapeutically effective amounts of the individual compounds and generally an acceptable pharmaceutical carrier, diluent or excipient, e.g. sterile water, physiological saline, bacteriostatic saline, i.e. saline containing about 0.9% mg/ml benzyl alcohol, phosphate-buffered saline, Hank's solution, Ringer's-lactate, lactose, dextrose, sucrose, trehalose, sorbitol, Mannitol, and the like. The compositions are preferably formulated as solution or suspension. Lyophilized or other dry-powder formulations, solid formulations, liposomal formulations or any other kind of formulation is also conceivable. The pharmaceutical compositions of present invention can be administered orally, subcutaneously, intramuscularly, pulmonary, by inhalation and/or through sustained release administrations. Preferably, the composition is administered subcutaneously.

The terms “therapeutically effective amount” or “therapeutic amount” are intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the term “therapeutically effective amount” as used herein means the quantity of a compound that results in the desired therapeutic and/or prophylactic effect without causing unacceptable side-effects. Particularly, the dosage a patient receives can be selected so as to achieve the blood sugar level or blood glucose level desired; the dosage a patient receives may also be titrated over time in order to reach a target blood glucose or blood sugar level. The dosage regimen utilizing the fusion protein as described herein is selected in accordance with a variety of factors including type, species, age, weight, body mass index, sex and medical condition of the patient; the severity of the condition to be treated; the potency of the compound chosen to be administered; the route of administration; the purpose of the administration; and the renal and hepatic function of the patient.

A typical dosage range is from about 0.01 mg per day to about 1000 mg per day. A preferred dosage range for each therapeutically effective compound is from about 0.1 mg per day to about 100 mg per day and a most preferred dosage range is from about 1.0 mg/day to about 10 mg/day, in particular about 1-5 mg/day.

In case of subsequent administration(s), the individual compounds (e.g. the fusion protein and optionally the anti-diabetic drug and optionally the DPP-IV inhibitor) are administered during a time period, in which the effect of the fusion protein and optionally the anti-diabetic drug and/or the DPP-IV inhibitor are still measurable e.g. in a “glucose tolerance test”, as e.g. shown in the Examples. The glucose tolerance test is a test to determine how quickly glucose is cleared from the blood after administration of glucose. The glucose is most often given orally (“oral glucose tolerance test” or “OGTT”). The time period for the subsequent administration of the individual compounds, in particular of the fusion protein, is usually within one hour, preferably, within half an hour, most preferably within 15 minutes, in particular within 5 minutes.

Generally, the application of the fusion protein or the pharmaceutical composition to a patient is one or several times per day, or one or several times a week, or even during longer time periods as the case may be. The most preferred application of the fusion protein or pharmaceutical composition of the present invention is a subcutaneous application one to three times per day, if applicable in a combined dose.

The term “Metabolic Syndrome” or “Metabolic Syndromes” as used herein, refers to one or more medical disorders which increase the risk of developing cardiovascular diseases and/or diabetes mellitus. Medical disorders increasing the risk of developing cardiovascular diseases and/or diabetes mellitus include but are not limited to dyslipidemia, fatty liver disease (FLD), dysglycemia, impaired glucose tolerance (IGT), obesity and/or adipositas.

Cardiovascular diseases are known in the art as a class of diseases that involve the heart or blood vessels (arteries and veins) such as but not limited to atherosclerosis.

Dyslipidemia is a condition wherein an abnormal amount of lipids (e.g. cholesterol, especially LDL cholesterol and/or fat such as triglycerides) is present in the blood. In developed countries, most dyslipidemias are hyperlipidemias; i.e. an elevation of lipids (e.g. triglycerides and/or LDL cholesterol) in the blood, often caused by diet and lifestyle. The prolonged elevation of insulin levels can also lead to dyslipidemia.

Fatty liver disease (FLD) is a reversible condition wherein large vacuoles of triglyceride fat accumulate in liver cells due to steatosis (i.e. abnormal retention of lipids within cells). FLD may have multiple causes however; predominately it is associated with excessive alcohol intake and obesity (with or without effects of insulin resistance).

Dysglycemia refers to an imbalance in the sugar metabolism/energy production mechanisms of the body. Diabetes mellitus is a metabolic disorder characterized by the presence of hyperglycemia. Impaired glucose tolerance (IGT) is a pre-diabetic state of dysglycemia that is associated with insulin resistance and increased risk of cardiovascular pathology and may precede type 2 diabetes mellitus by many years.

Obesity is a medical condition in which excess body fat has accumulated to the extent that it may have an adverse effect on health, leading to reduced life expectancy and/or increased health problems.

The terms “protein” and “polypeptide” are used interchangeably herein and refer to any peptide-linked chain of amino acids, regardless of length or post-translational modification. Proteins usable in the present invention (including protein derivatives, protein variants, protein fragments, protein segments, protein epitopes and protein domains) can be further modified by chemical or biological modification. This means such a biologically or chemically modified polypeptide comprises other chemical groups than the 20 naturally occurring amino acids. Examples of such other chemical groups include without limitation glycosylated amino acids, phosphorylated amino acids or covalent attachment of amino-acid chains e.g. for stabilization of the protein/polypeptide (such as attachment of, e.g. rPEG, XTEN or PAS). Modification of a polypeptide may provide advantageous properties as compared to the parent polypeptide, e.g. one or more of enhanced stability, increased biological half-life, or increased water solubility. Chemical modifications applicable to the variants usable in the present invention include without limitation: PEGylation, glycosylation of non-glycosylated parent polypeptides, or the modification of the glycosylation pattern present in the parent polypeptide, rPEGylation, XTENylation or PASylation.

The term “XTEN” and/or “XTENylation” refers to largely unstructured recombinant polypeptides comprised of the amino acids A, E, G, P, S and T. XTEN can have a length of about 864 amino acids but can also be shorter (e.g. fragments of the 864 amino acid long polypeptides according to WO2010091122 A1). The term XTENylation refers to the fusion of XTEN with a target therapeutic protein (the “payload”). As used herein, XTEN can be fused to a linker, to the GLP-1 R agonist, and/or to the FGF-21 compound or can also be used as a linker or part of a linker between two protein moieties of present fusion proteins. XTENylation serves to increase the serum-half-life of the therapeutic protein (i.e. herein, the fusion protein of present invention). The term “XTEN” and/or “XTENylation” also refers to an unstructured recombinant polypeptide (URP) comprising at least 40 contiguous amino acids, wherein (a) the sum of glycine (G), aspartate (D), alanine (A), serine (S), threonine (T), glutamate (E) and proline (P) residues contained in the URP, constitutes at least 80% of the total amino acids of the unstructured recombinant polypeptide, and the remainder, when present, consists of arginine or lysine, and the remainder does not contain methionine, cysteine, asparagine, and glutamine.

The term “PEG” and/or “PEGylation” refers to the covalent attachment of polyethylene glycol (PEG) polymer chains to a biopharmaceutical protein of interest such as the present invention (comprising a GLP-1R agonist and a FGF-21 compound). The covalent attachment of PEG to a biopharmaceutical protein of interest can mask the agent from the host's immune system (reduced immunogenicity and antigenicity), and increase the hydrodynamic size of the biopharmaceutical protein of interest which prolongs its circulation time by reducing renal clearance (and so modulates the pharmacokinetic of the biopharmaceutical protein of interest). As used herein, PEG can be covalently attached to a linker, to the GLP-1 R agonist, and/or to the FGF-21 compound or can also be used as a linker or part of a linker between two protein moieties of present fusion proteins.

The term “PAS” and/or “PASylation” refers to the genetic fusion of a biopharmaceutical protein of interest such as the present fusion protein with a conformationally disordered polypeptide sequence composed of the amino acids Pro, Ala and Ser (hence the term “PASylation”). As used herein, PAS can be fused to a linker, to the GLP-1 R agonist, and/or to the FGF-21 compound or can also be used as a linker or part of a linker between two protein moieties of present fusion proteins. PASylation serves to Increase the serum-half life of the protein of interest, e.g. the fusion protein (for reference, see WO2008155134 Al). The term “PAS” and/or “PASylation” also refers to a biologically active protein comprising at least two domains, wherein (a) a first domain of said two domains comprises an amino acid sequence having and/or mediating said biological activity; and (b) a second domain of said at least two domains comprises an amino acid sequence consisting of at least about 100 amino acid residues forming random coil conformation and wherein said second domain consists of alanine, serine and proline residues, whereby said random coil conformation mediates an increased in vivo and/or in vitro stability of said biologically active protein. In a preferred embodiment, said second domain comprises the amino acid sequence selected from the group consisting of:

(SEQ ID NO: 95)
ASPAAPAPASPAAPAPSAPA;
(SEQ ID NO: 96)
AAPASPAPAAPSAPAPAAPS;
(SEQ ID NO: 97)
APSSPSPSAPSSPSPASPSS;
(SEQ ID NO: 98)
SAPSSPSPSAPSSPSPASPS;
(SEQ ID NO: 99)
SSPSAPSPSSPASPSPSSPA;
(SEQ ID NO: 100)
AASPAAPSAPPAAASPAAPSAPPA;
(SEQ ID NO: 101)
ASAAAPAAASAAASAPSAAA.

The PASylation sequence may contain one or more site(s) for covalent modification. rPEG are polypeptides with PEG-like properties having increased hydrodynamic radius, that are genetically fused to biopharmaceuticals. As used herein, rPEG can be fused to a linker, to the GLP-1 R (glucagon-like peptide-1 receptor) agonist, and/or to the FGF-21 (fibroblast growth factor 21) compound or can also be used as a linker or part of a linker between two protein moieties of present fusion proteins.

Elastin-like polypeptides (ELPs) are a class of stimulus responsive biopolymers whose physicochemical properties and biocompatibility are suitable for in vivo applications, such as drug delivery and tissue engineering. The lower critical solution temperature (LCST) behavior of ELPs allows them to be utilized as soluble macromolecules below their LOST, or as self-assembled nano-scale particles such as micelles, micron-scale coacervates, or viscous gels above their LOST, depending on the ELP architecture. As each ELP sequence is specified at its genetic level, functionalization of an ELP with peptides and proteins is to accomplish by the fusion of a gene encoding an ELP with that of the peptide or protein of interest. Protein ELP fusions, where the appended protein serves a therapeutic or targeting function, are suitable for applications in which the ELP can improve the systemic pharmacokinetics and biodistribution of the protein, or can be used as an injectable depot for sustained, local protein delivery. The repeat unit in ELPs is a pentapeptide of (Val-Pro-Gly-X-Gly), where X is a ‘guest residue’ that can be any amino acid other than proline (Hassouneh et al., Methods Enzymol. 2012; 502: 215-237). As used herein, ELPs can be covalently attached to a linker, to the GLP-1 R agonist, and/or to the FGF-21 compound or can also be used as a linker or part of a linker between two protein moieties of present fusion proteins.

In the context of the different aspects of present invention, the term “peptide” refers to a short polymer of amino acids linked by peptide bonds. It has the same chemical (peptide) bonds as proteins, but is commonly shorter in length. The shortest peptide is a dipeptide, consisting of two amino acids joined by a single peptide bond. There can also be a tripeptide, tetrapeptide, pentapeptide, etc. Preferably, the peptide has a length of up to 8, 10, 12, 15, 18 or 20 amino acids. A peptide has an amino end and a carboxyl end, unless it is a cyclic peptide.

In the context of the different aspects of present invention, the term “polypeptide” refers to a single linear chain of amino acids bonded together by peptide bonds and preferably comprises at least about 21 amino acids. A polypeptide can be one chain of a protein that is composed of more than one chain or it can be the protein itself if the protein is composed of one chain.

In the context of the different aspects of present invention, the term “protein” refers to a molecule comprising one or more polypeptides that resume a secondary and tertiary structure and additionally refers to a protein that is made up of several polypeptides, i.e. several subunits, forming quaternary structures. The protein has sometimes non-peptide groups attached, which can be called prosthetic groups or cofactors.

In the context of present invention, the primary structure of a protein or polypeptide is the sequence of amino acids in the polypeptide chain. The secondary structure in a protein is the general three-dimensional form of local segments of the protein. It does not, however, describe specific atomic positions in three-dimensional space, which are considered to be tertiary structure. In proteins, the secondary structure is defined by patterns of hydrogen bonds between backbone amide and carboxyl groups. The tertiary structure of a protein is the three-dimensional structure of the protein determined by the atomic coordinates. The quaternary structure is the arrangement of multiple folded or coiled protein or polypeptide molecules molecules in a multi-subunit complex. The terms “amino acid chain” and “polypeptide chain” are used synonymously in the context of present invention.

The terms “nucleic acid” or “nucleic acid molecule” are used synonymously and are understood as single or double-stranded oligo- or polymers of deoxyribonucleotide or ribonucleotide bases or both. Typically, a nucleic acid is formed through phosphodiester bonds between the individual nucleotide monomers. In the context of the present invention, the term nucleic acid includes but is not limited to ribonucleic acid (RNA) and deoxyribonucleic acid (DNA) molecules. The depiction of a single strand of a nucleic acid also defines (at least partially) the sequence of the complementary strand. The nucleic acid may be single or double stranded, or may contain portions of both double and single stranded sequences. The nucleic acid may be obtained by biological, biochemical or chemical synthesis methods or any of the methods known in the art. As used herein, the term “nucleic acid” comprises the terms “polynucleotide” and “oligonucleotide”.

In the context of the different aspects of present invention, the term nucleic acid comprises cDNA, genomic DNA, recombinant DNA, cRNA and mRNA. A nucleic acid may consist of an entire gene, or a portion thereof, the nucleic acid may also be a microRNA (miRNA) or small interfering RNA (siRNA). MiRNAs are short ribonucleic acid (RNA) molecules, on average only 22 nucleotides long, found in all eukaryotic cells. MircoRNAs (miRNAs) are post-transcriptional regulators that bind to complementary sequences on target messenger RNA transcripts (mRNAs), usually resulting in translational repression and gene silencing. Small interfering RNAs (siRNAs), sometimes known as short interfering RNA or silencing RNA, are short ribonucleic acid (RNA molecules), between 20-25 nucleotides in length. They are involved in the RNA interference (RNAi) pathway, where they interfere with the expression of specific genes. The nucleic acid can also be an artificial nucleic acid. Artificial nucleic acids include polyamide or peptide nucleic acid (PNA), morpholino and locked nucleic acid (LNA), as well as glycol nucleic acid (GNA) and threose nucleic acid (TNA). Each of these is distinguished from naturally-occurring DNA or RNA by changes to the backbone of the molecule.

The nucleic acids, can e.g. be synthesized chemically, e.g. in accordance with the phosphotriester method (see, for example, Uhlmann, E. & Peyman, A. (1460) Chemical Reviews, 90, 543-584). Aptamers are nucleic acids which bind with high affinity to a polypeptide. Aptamers can be isolated by selection methods such as SELES (see e.g. Jayasena (1469) Clin. Chem., 45, 1628-50; Klug and Famulok (1464) M. Mol. Biol. Rep., 20, 97-107; U.S. Pat. No. 5,582,981) from a large pool of different single-stranded RNA molecules. Aptamers can also be synthesized and selected in their mirror-image form, for example as the L-ribonucleotide (Nolte et al. (1466) Nat. Biotechnol., 14, 1116-9; Klussmann et al. (1466) Nat. Biotechnol., 14, 1112-5). Forms which have been isolated in this way enjoy the advantage that they are not degraded by naturally occurring ribonucleases and, therefore, possess greater stability. Nucleic acids may be degraded by endonucleases or exonucleases, in particular by DNases and RNases which can be found in the cell. It is, therefore, advantageous to modify the nucleic acids in order to stabilize them against degradation, thereby ensuring that a high concentration of the nucleic acid is maintained in the cell over a long period of time (Beigelman et al. (1465) Nucleic Acids Res. 23:3989-94; WO 95/11910; WO 98/37240; WO 97/29116). Typically, such stabilization can be obtained by introducing one or more internucleotide phosphorus groups or by introducing one or more non-phosphorus internucleotides. Suitably modified internucleotides are compiled in Uhlmann and Peyman (1460), supra (see also Beigelman et al. (1465) Nucleic Acids Res. 23:3989-94; WO 95/11910; WO 98/37240; WO 97/29116). Modified internucleotide phosphate radicals and/or non-phosphorus bridges in a nucleic acid which can be employed in one of the uses according to the invention contain, for example, methyl phosphonate, phosphorothioate, phosphoramidate, phosphorodithioate and/or phosphate esters, whereas non-phosphorus internucleotide analogues contain, for example, siloxane bridges, carbonate bridges, carboxymethyl esters, acetamidate bridges and/or thioether bridges. It is also the intention that this modification should improve the durability of a pharmaceutical composition which can be employed in one of the uses according to the invention,

The invention will now be described in more detail in the specific description.

Specific Description

In the following, the different aspects and embodiments of present invention will be described in detail.

The different aspects, preferred aspects and embodiments of present invention can be combined with each other unless explicitly stated to the contrary. Any of the embodiments of any of the aspects or preferred aspects of present invention can be combined with any of the embodiments of any of the other aspects or preferred aspects of present invention unless explicitly stated to the contrary.

In a first aspect, present invention concerns a fusion protein comprising the polypeptide with structure A-B-C or C-B-A or B-A-C or B-C-A or A-C-B or C-A-B or A-B-C-B-C or A-C-B or A-B-C-B or A-C-B-C, wherein

A is a GLP-1 R (glucagon-like peptide-1 receptor) agonist and

C is an FGF-21 (fibroblast growth factor 21) compound and

B is a Linker comprising about 0, 1 to 1000 amino acids.

The components A-B-C are preferably arranged from the amino-terminus (N-terminus) to the carboxy-terminus (C-terminus) of the fusion protein, so that the fusion protein has the structure A-B-C or C-B-A or B-A-C or B-C-A or A-C-B or C-A-B or A-B-C-B-C or A-C-B or A-B-C-B or A-C-B-C. According to a preferred embodiment, the components have the arrangement A-B-C from the N-terminus to the C-terminus of the fusion protein.

The FGF-21 compound according to the first and the other aspect of present invention can be any polypeptide having FGF-21 activity and preferably is an FGF-21 compound and preferably a FGF-21 compound according to SEQ ID NO: 3 as herein described.

According to one embodiment of the first and the other aspects of present invention, the FGF-21 compound is native FGF-21 or an FGF-21 mimetic or FGF-21 according to SEQ ID NO: 3. According to a preferred embodiment of the first and the other aspects of present invention, the FGF-21 mimetic can e.g. be a protein having at least about 96% amino acid sequence identity to the amino acid sequence shown in SEQ ID NO: 3 and having FGF-21 activity, or an FGF-21 fusion protein with FGF-21 activity or a FGF-21 conjugate having FGF-21 activity. The FGF-21 mimetic can e.g. be an FGF-21 mutein, an FGF-21-Fc fusion protein, an FGF-21-HSA fusion protein and/or a PEGylated FGF-21.

The GLP-1 R agonist comprised in the fusion protein of the first and the other aspects of present invention can be any polypeptide having GLP-1 receptor-agonistic action and preferably is a GLP-1 R agonist as herein described. In one embodiment of the fusion protein of present invention, the GLP-1R agonist a bioactive GLP-1, a GLP-1 analogue or a GLP-1 substitute. In preferred embodiments of the fusion protein of present invention, the GLP-1R agonist is e.g. GLP-1(7-37), GLP-1(7-36)amide, exendin-4, liraglutide, CJC-1131, albugon, albiglutide, exenatide, exenatide-LAR, oxyntomodulin, lixisenatide, geniproside, or a short peptide with GLP-1R agonistic activity.

In another preferred embodiment of the first and the other aspects of present invention, A is an FGF-21 mutein and C is exenatide, exendin-4 or lixisenatide. In another preferred embodiment of the fusion protein of present invention, A is an FGF-21 mutein and C is exenatide, exendin-4 or lixisenatide and B is IEGR.

In another preferred embodiment of the first and the other aspects of present invention, A is a FGF-21 compound according to SEQ ID NO: 3 and C is exenatide, exendin-4 or lixisenatide. In another preferred embodiment of the fusion protein of present invention, A is an FGF-21 mutein and C is exenatide, exendin-4 or lixisenatide and B is IEGR.

In another preferred embodiment of the first and the other aspects of present invention, A is an FGF-21 mutein, comprising SEQ ID NO: 2 or 102. In another preferred embodiment of the fusion protein of present invention, C is exenatide.

In another preferred embodiment of the first and the other aspects of present invention, A is a FGF-21 compound according to. SEQ ID NO: 3.

In another preferred embodiment of the first and the other aspects of present invention, A is an FGF-21 mutein, comprising SEQ ID NO: 2 or 102 and C is exenatide. In another preferred embodiment of the fusion protein of present invention, A is an FGF-21 mutein, comprising SEQ ID NO: 102 and the linker B is IEGR. In another preferred embodiment of the fusion protein of present invention, the linker B is IEGR and C is exenatide.

In another preferred embodiment of the first and the other aspects of present invention, A is an FGF-21 compound according to SEQ ID NO: 3 and C is exenatide. In another preferred embodiment of the fusion protein of present invention, A is an FGF-21 compound according to SEQ ID NO: 3 and the linker B is IEGR. In another preferred embodiment of the fusion protein of present invention, the linker B is IEGR and C is exenatide.

In another preferred embodiment of the first and the other aspects of present invention, A is an FGF-21 mutein, comprising SEQ ID NO: 2 or 102, the linker B is IEGR and C is exenatide.

In another preferred embodiment of the first and the other aspects of present invention, A is an FGF-21 compound according to SEQ ID NO: 3, the linker B is IEGR and C is exenatide.

The fusion protein can also comprise further components in addition to components A, B and C. In one embodiment, the fusion protein comprises one or more moieties D being covalently attached to the entry site(s) for covalent modification of the linker. The covalently attached moiety or moieties D can e.g. confer increased half-life or stability to the fusion protein, target the protein to some molecular or cellular target in the patient's body, attract the immune system, increase efficacy of the fusion protein etc. The attached moiety can be a peptide/polypeptide, nucleic acid, carbohydrate, fatty acid, organic molecule or combination thereof. According to one embodiment, the moiety or moieties D is or are selected from the list consisting of:

a) a targeting unit such as an antibody or protein-binding scaffold or aptamer

b) a protein-stabilizing unit such as a hydroxyethyl starch derivative (HES) or a polyethylenglycol or derivative thereof (PEG or PEG derivative);

c) a fatty acid;

d) a carbohydrate.

The fusion protein of present invention can also comprise further components, such as a tag for protein-purification; e.g. a His-tag. In one embodiment, the tag is terminally (N- or C-terminally) attached to the fusion protein.

In a second aspect, present invention concerns the fusion protein of present invention for use as a medicament.

In one embodiment of the second and the other aspects of present invention, the medical use is a use in the treatment of a disease or disorder in which the increase of FGF-21 receptor autophosphorylation or the increase of FGF-21 efficacy is beneficial for the curing, prevention or amelioration of the disease.

In another embodiment of the second and the other aspects of present invention, the medical use is a use in the treatment of a cardiovascular disease and/or diabetes mellitus and/or at least one metabolic syndrome which increases the risk of developing a cardiovascular disease and/or for use in the treatment of diabetes mellitus, preferably Type 2-diabetes.

In another embodiment of the second and the other aspects of present invention, the medical use is a use in the lowering of plasma glucose levels, in the lowering of the lipid content in the liver, for use in treating hyperlipidemia, for use in treating hyperglycemia, for use in increasing the glucose tolerance, for use in decreasing insulin tolerance, for use in increasing the body temperature, and/or for use in reducing weight.

In another embodiment of the second and the other aspects of present invention, the medical use further involves administration of at least one anti-diabetic drug and/or at least one DPP-IV (dipeptidyl peptidase-4) inhibitor. In this embodiment, the fusion protein and the anti diabetic drug and/or the DPP-IV inhibitor can be administered simultaneously or subsequently with the fusion protein. This means, that the following administration regimes are conceivable: The DPP-IV inhibitor is administered simultaneously with the fusion protein, the anti-diabetic drug is administered simultaneously with the fusion protein, the DPP IV-inhibitor and the anti-diabetic drug are administered simultaneously with the fusion protein, the DPP-IV inhibitor is administered subsequently with (i.e. prior or after) administration of the fusion protein, the anti-diabetic drug is administered subsequently with (i.e. prior or after) administration of the fusion protein, the DPP-IV inhibitor and the anti-diabetic drug are administered subsequently with (i.e. prior or after) administration of the fusion protein, the DPP-IV inhibitor is administered simultaneously with the fusion protein whereas the anti-diabetic drug is administered subsequently with (i.e. prior or after) administration of the fusion-protein comprising composition, the DPP-IV inhibitor is administered subsequently with (i.e. prior or after) the fusion protein whereas the anti-diabetic drug is administered simultaneously with administration of the fusion protein.

The anti-diabetic drug of the second and the other aspects of present invention can be any agent or drug with anti-diabetic activity and preferably any anti-diabetic drug as described herein. In some embodiments of the first and the other aspects of present invention, the anti-diabetic drug is metformin, a thiazolidinedione, a sulphonylurea, insulin or a combination of two, three or four of these anti-diabetic drugs.

The DPP-IV inhibitor of the second and the other aspects of present invention can be any agent or drug with DPP-IV antagonistic or inhibitory action. In some embodiments of the first and the other aspects of present invention, the DPP-IV inhibitor is sitagliptin, vildagliptin, saxagliptin, linagliptin, adogliptin or berberine or a combination of two, three, four, five or six of these DPP-IV inhibitors.

Further embodiments and particulars of the second aspect can also be taken from the other aspects herein described, the general description, the examples or any other section hereof. Embodiments and preferred embodiments of the fusion protein of the second aspect are described, in detail, in the section dealing with the first aspect of present invention and are also described in the general section, the definitions section and the Examples section herein. Further particulars concerning the medical use, indication, patient population, administration or dosage regimen can e.g. be taken from the description of the sixth, seventh or eighth aspect of present invention described herein

In a third aspect, the present invention concerns a pharmaceutical composition comprising the fusion protein of the present invention together with a pharmaceutically acceptable excipient.

The fusion proteins herein described and particularly in the context of the first, third and the other aspects of present invention can e.g. be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as but not limited to those derived from sodium, potassium, ammonium, calcium, ferric hydroxides, isopropylamine, triethylamine, 2-ethylamino ethanol, histidine, procaine, and the like. Further embodiments and particulars of the third aspect can also be taken from the other aspects herein described, the general description, the examples or any other section hereof. Embodiments and preferred embodiments of the fusion protein of the second aspect are described, in detail, in the section dealing with the first aspect of present invention and are also described in the general section, the definitions section and the Examples section herein.

In a fourth aspect, present invention concerns the fusion protein of present invention or a pharmaceutical composition comprising the fusion protein of the present invention together with a pharmaceutically acceptable excipient for use as a medicament.

In one embodiment of the fourth and the other aspects of present invention, the pharmaceutical composition is for use in the treatment of a disease or disorder in which the increase of FGF-21 receptor autophosphorylation or the increase of FGF-21 efficacy is beneficial for the curing, prevention or amelioration of the disease.

In another embodiment of the fourth and the other aspects of present invention, the pharmaceutical composition is for use in the treatment of a cardiovascular disease and/or diabetes mellitus and/or at least one metabolic syndrome which increases the risk of developing a cardiovascular disease and/or for use in the treatment of diabetes mellitus, preferably Type 2-diabetes.

In another embodiment of the fourth and the other aspects of present invention, the pharmaceutical composition is for use in the lowering of plasma glucose levels, in the lowering of the lipid content in the liver, for use in treating hyperlipidemia, for use in treating hyperglycemia, for use in increasing the glucose tolerance, for use in decreasing insulin tolerance, for use in increasing the body temperature, and/or for use in reducing weight.

In another embodiment of the fourth and the other aspects of present invention, the medical use of the pharmaceutical composition further involves administration of at least one anti-diabetic drug and/or at least one DPP-IV (dipeptidyl peptidase-4) inhibitor. In this embodiment, the anti diabetic drug and optionally the DPP-IV inhibitor or both can e.g. be administered simultaneously or subsequently with the pharmaceutical composition comprising the fusion protein. This means, that the following administration regimes are conceivable: The DPP-IV inhibitor is administered simultaneously with the fusion protein, the anti-diabetic drug is administered simultaneously with the fusion protein, the DPP IV-inhibitor and the anti-diabetic drug are administered simultaneously with the fusion protein, the DPP-IV inhibitor is administered subsequently with (i.e. prior or after) administration of the fusion protein, the anti-diabetic drug is administered subsequently with (i.e. prior or after) administration of the fusion protein, the DPP-IV inhibitor and the anti-diabetic drug are administered subsequently with (i.e. prior or after) administration of the fusion protein, the DPP-IV inhibitor is administered simultaneously with the fusion protein-comprising pharmaceutical composition whereas the anti-diabetic drug is administered subsequently with (i.e. prior or after) administration of the fusion-protein comprising composition, the DPP-IV inhibitor is administered subsequently with (i.e. prior or after) the fusion protein-comprising pharmaceutical composition whereas the anti-diabetic drug is administered simulataneously with administration of the fusion-protein comprising composition.

The anti-diabetic drug for use in the fourth and the other aspects of present invention can be any anti-diabetic drug as described above for the first aspect of present invention and is preferably metformin, a thiazolidinedione, a sulphonylurea or insulin or a combination of two, three or four of these anti-diabetic drugs.

The DPP-IV inhibitor for use in the fourth and the other aspects of present invention can be any anti-diabetic drug as described above for the first aspect of present invention and is preferably sitagliptin, vildagliptin, saxagliptin, linagliptin, adogliptin or berberine or a combinaiton of two, three, four, five or six of these DPP IV-inhibitors.

In the fourth aspect or any of the other aspects of present invention, the fusion protein, the anti-diabetic drug, and the DPP-IV inhibitor can be comprised in one formulation or contained in separate formulations.

In one embodiment of the fourth and the other aspects of present invention, the fusion protein and the anti-diabetic agent are comprised in one formulation. In another embodiment of the second and the other aspects of present invention, the fusion protein and the anti-diabetic agent are comprised in separate formulations.

In one embodiment of the fourth or any other aspect of present invention, the fusion protein and the DPP-IV inhibitor are combined in one formulation. In another embodiment of the second and the other aspects of present invention, the fusion protein and the DPP-IV inhibitor are contained in separate formulations.

In one embodiment of the fourth or any other aspect of present invention, the anti-diabetic drug and the DPP-IV inhibitor are combined in one formulation. In another embodiment of the second and the other aspects of present invention, the anti-diabetic drug and the DPP-IV inhibitor are contained in separate formulations.

In one embodiment of the fourth or any other aspect of present invention, the anti-diabetic drug and the DPP-IV inhibitor are combined in one formulation and the fusion protein is comprised in a separate formulation. In another embodiment of the second and the other aspects of present invention, the anti-diabetic drug and the fusion protein are comprised in one formulation and the DPP-IV inhibitor is comprised in a separate formulation. In another aspect of the second and the other aspects of present invention, the fusion protein and the DPP-IV inhibitor are comprised in one formulation and the anti-diabetic drug is comprised in a separate formulation.

In another embodiment of the fourth or any other aspect of present invention, the DPP-IV inhibitor and the anti-diabetic drug(s) and the fusion protein are all comprised in separate formulations. In yet another embodiment of the second or any other aspect of present invention, the DPP-IV inhibitor and the anti-diabetic drug(s) and the fusion protein are combined in one formulation.

Further embodiments and particulars of the fourth aspect can also be taken from the other aspects herein described. E.g. further particulars concerning the medical use, indication, patient population, administration or dosage regimen can be taken from the description of the second, sixth, seventh or eighth aspect of present invention described herein. Further particulars concerning the fusion protein can e.g. be taken from the description of the first aspect, the general definitions section, the examples or figures.

In a fifth aspect, present invention concerns an article of manufacture comprising

a) the fusion protein or the pharmaceutical composition of the present invention and

b) a container or packaging material.

Certain embodiments concerning the fusion proteins for use in the context of the article of manufacture of the fifth aspect can be taken from the above description of the first aspect, from the general description, the definitions section or the Examples section. Certain embodiments concerning the pharmaceutical compositions for use in the context of the article of manufacture of the fifth aspect can be taken from the above description of the third aspect, from the general description, the definitions section or the Examples section. Certain embodiments concerning the medical use of the article of manufacture of the fifth aspect or the indication or patient population listed on the data carrier can be taken from the above description of the second, fourth or sixth to eighth aspect, from the general description, the definitions section or the Examples section.

Further embodiments will be described in the following:

In some embodiments the article of manufacture can additionally comprise

c) a pharmaceutical composition comprising a DPP-IV inhibitor, or

d) a pharmaceutical composition comprising an anti-diabetic drug, or

e) both (a and b).

The article of manufacture can further comprise one or more data carriers. The data carrier can be any carrier of data that are beneficial for use of the article of manufacture. The data carrier can e.g. be a label, a packaging insert, a digital data carrier such as a chip, a bar code etc. The information contained in or on the data carrier can e.g. be one or more of the following:
a) Reference to a medical use according to any one of the aspects of present invention (e.g. the first or second aspect) or as described in the general or definitions section or in the Examples section, and/or reference to a method of treatment according to any one of the aspects of present invention (e.g. the sixth, seventh, eighth or ninth aspect),
b) Storage conditions (e.g. temperature, humidity, exposure to light) of the article of manufacture or the components thereof (eg. storage conditions of the buffers, storage conditions of the therapeutic agents or the pharmaceutical compositions or unit dosage forms comprising the therapeutic agents (i.e. comprising the fusion protein, the DPP-IV inhibitor or the anti-diabetic agent or two or three of these)
c) Lot number or batch number of the article of manufacture
d) Composition of the article of manufacture and optionally the components thereof
e) Handling instructions of the article of manufacture and optionally its components
f) Expiry date of the article of manufacture (preferably if stored under the indicated storage conditions), wherein the expiry date can refer to the expiry date of the article of manufacture in general, individual of its components or to the article of manufacture or individual of its components after opening up of the package or packaging material comprising one or more of the components (or both).

The article of manufacture can further comprise one or more devices for application of the fusion protein or the pharmaceutical composition comprising the fusion protein and and instructions for use of the device. If the device is a pre-filled device, the device preferably contains a label indicating the content and more preferably also the expiry date.

According to one embodiment of the fifth aspect of present invention, the article of manufacture comprises one or more of the following components:
a) one or more unit dosage forms comprising the fusion protein
b) one or more unit dosage forms comprising the anti-diabetic drug
c) one or more unit dosage forms comprising the DPP-IV inhibitor
d) a data carrier, the data carrier preferably comprising a label or package insert;
e) a device for application of the fusion protein such as a syringe and instructions for use of the device.

The fusion protein in the article of manufacture can e.g. be formulated as dry formulation for dissolution, preferably comprised in a hermetically sealed container such as a vial, an ampoule or sachette

The fusion protein in the article of manufacture can also be formulated as liquid formulation preferably comprised in a hermetically sealed container such as a vial, a sachette, a pre-filled syringe, a pre-filled autoinjector or a cartridge for a reusable syringe or applicator.

The article of manufacture of present invention can also comprise one or more unit dosage forms of the anti-diabetic drug as tablet or capsule or other formulation for oral administration in a hermetically sealed container or blister.

The article of manufacture of present invention can also comprise one or more unit dosage forms of the DPP-IV inhibitor as tablet or capsule or other formulation for oral administration in a hermetically sealed container or blister

The container or blister containing the unit dosage form(s) comprising the fusion protein or any other of the therapeutic agents or pharmaceutical formulations suitably contains a label indicating
a) the content (such as the identity and quantity of active ingredient and possibly any excipient) and preferably also
b) the expiry date and possibly also
c) the storage conditions of the active ingredients (the fusion protein and/or the DPP-IV inhibitor and/or the anti-diabetic drug) or the article of manufacture.

According to one embodiment, the article of manufacture comprises sufficient unit dosage forms of the fusion protein and preferably also of the anti-diabetic drug or DPP IV-inhibitor or sufficient unit dosage forms of the fusion protein and anti-diabetic drug and DPP IV-inhibitor, for one single, for a two-week (i.e. 14-day) treatment, for a four week (i.e, 28-day) treatment or for a one-month treatment with fusion protein and preferably the anti-diabetic drug or DPP IV-inhibitor or with fusion protein and the anti-diabetic drug and the DPP IV-inhibitor.

According to another embodiment, the article of manufacture comprises sufficient unit dosage forms of the fusion protein and optionally of the anti-diabetic drug or the DPP-IV inhibitor or both for a daily administration regime and more preferably for a daily administration regime in a one-day, one-week, two-week or four-week/one month treatment period.

The device or devices optionally contained within the article of manufacture can be any device for application of any or all of the therapeutic agents (fusion protein, DPP-IV inhibitor, anti-diabetic agent) can e.g. be a syringe or another type of injection device. This is particularly suitable if the active agent(s) is or are formulated as injection solution(s) or dry-powder formulation(s) for dissolution and later injection application In this case it can be suitable if the device or syringe is pre-filled or suitable for subcutaneous injection or both pre-filled and suitable for subcutaneous injection.

In a sixth aspect, the present invention concerns a method of treating a disease or disorder of a patient, in which the increase of FGF-21 receptor autophosphorylation or in which the increase of FGF-21 efficacy is beneficial for the curing, prevention or amelioration of the disease or disorder, wherein the method comprises administration to the patient of a fusion protein or the pharmaceutical composition of present invention.

In a seventh aspect, the present invention concerns a method of treating a cardiovascular disease and/or diabetes mellitus and/or at least one metabolic syndrome which increases the risk of developing a cardiovascular disease and/or diabetes mellitus, preferably Type 2-diabetes in a patient comprising the administration to the patient of a fusion protein or the pharmaceutical composition of present invention.

In an eighth aspect, the present invention concerns a method of lowering plasma glucose levels, of lowering the lipid content in the liver, of treating hyperlipidemia, of treating hyperglycemia, of increasing the glucose tolerance, of decreasing insulin tolerance, of increasing the body temperature, and/or of reducing weight of a patient comprising the administration to the patient of a fusion protein or the pharmaceutical composition of present invention.

Certain embodiments concerning the fusion proteins for use in the context of methods of treatment can be taken from the above description of the first aspect, from the general description, the definitions section or the Examples section. Certain embodiments concerning the pharmaceutical compositions for use in the context of the herein described methods of treatment can be taken from the above description of the third aspect, from the general description, the definitions section or the Examples section. Certain embodiments concerning the medical use of the herein described methods of treatment can be taken from the above description of the or second aspect, from the general description, the definitions section or the Examples section. Further embodiments of the herein described methods of treatment will be described in the following:

In one embodiment of the sixth, seventh or eighth aspect, the method further comprises the administration of at least one antidiabetic drug or the administration of a dipeptidyl peptidase-4 (DPP-IV) inhibitor or both.

In another embodiment of the sixth, seventh or eighth aspect of present invention, the method of treatment further involves administration of at least one anti-diabetic drug and/or at least one DPP-IV (dipeptidyl peptidase-4) inhibitor. In this embodiment, the anti diabetic drug and optionally the DPP-IV inhibitor or both can e.g. be administered simultaneously or subsequently with the pharmaceutical composition comprising the fusion protein. This means, that the following administration regimes are conceivable: The DPP-IV inhibitor is administered simultaneously with the fusion protein, the anti-diabetic drug is administered simultaneously with the fusion protein, the DPP IV-inhibitor and the anti-diabetic drug are administered simultaneously with the fusion protein, the DPP-IV inhibitor is administered subsequently with (i.e. prior or after) administration of the fusion protein, the anti-diabetic drug is administered subsequently with (i.e. prior or after) administration of the fusion protein, the DPP-IV inhibitor and the anti-diabetic drug are administered subsequently with (i.e. prior or after) administration of the fusion protein, the DPP-IV inhibitor is administered simultaneously with the fusion protein-comprising pharmaceutical composition whereas the anti-diabetic drug is administered subsequently with (i.e. prior or after) administration of the fusion-protein comprising composition, the DPP-IV inhibitor is administered subsequently with (i.e. prior or after) the fusion protein-comprising pharmaceutical composition whereas the anti-diabetic drug is administered simultaneously with administration of the fusion-protein comprising composition.

The anti-diabetic drug for use in the sixth, seventh or eighth aspect of present invention can be any anti-diabetic drug as described above for the first aspect of present invention and is preferably metformin, a thiazolidinedione, a sulphonylurea or insulin or a combination of two, three or four of these anti-diabetic drugs.

The DPP-IV inhibitor for use in the sixth, seventh or eighth aspect of present invention can be any anti-diabetic drug as described above for the first aspect of present invention and is preferably sitagliptin, vildagliptin, saxagliptin, linagliptin, adogliptin or berberine or a combinaiton of two, three, four, five or six of these DPP IV-inhibitors.

In one embodiment of the sixth, seventh or eighth aspect of present invention, the fusion protein is administered to the patient at the same time as the anti-diabetic drug or the DPP-IV inhibitor or both.

In another embodiment of the sixth, seventh or eighth aspect of present invention, the fusion protein is administered to the patient before or after the anti-diabetic drug or the DPP-IV inhibitor or both.

In one embodiment of the sixth, seventh or eighth aspect of present invention the metabolic syndrome is selected from the group consisting of dysiipidemia, fatty liver disease (FLD), dysglycemia, impaired glucose tolerance (IGT), obesity, adipositas, and Type 2-diabetes.

The cardiovascular disease of the sixth, seventh or eighth aspect can e.g. be atherosclerosis.

The patient to be treated in the context of the sixth, seventh or eighth aspect of present invention is preferably selected from the group consisting of: a Type 1-diabetic patient, a-Type 2-diabetic patient, a diet-treated Type 2-diabetic patient, a sulfonylurea-treated Type 2-diabetic patient, a far-advanced stage Type 2-diabetic patient, and a long-term insulin-treated Type 2-diabetic patient.

In some embodiments of the sixth, seventh or eighth aspect of present invention, the plasma glucose levels are lowered, the lipid content in the liver is lowered, the glucose tolerance is increased, the insulin tolerance is increased, the body temperature is increased, and/or the weight is reduced in a diabetic patient, preferably selected from the group consisting of a Type 1-diabetic patient, a Type 2-diabetic patient, in particular a diet-treated Type 2-diabetic patient, a sulfonylurea-treated Type 2-diabetic patient, a far-advanced stage Type 2-diabetic patient and/or a long-term insulin-treated Type 2-diabetic patient. According to a preferred embodiment, the patient is a mammal and particularly a human being.

In the context of the different medical uses and methods of treatment of the first, second, fifth, sixth, seventh or eighth aspect of present invention, it is suitable if a therapeutically effective amount of the fusion protein or pharmaceutical composition and optionally the anti-diabetic drug or the DPP IV-inhibitor or both is administered to the patient.

In the context of the different medical uses and methods of treatment of the first, second, fifth, sixth, seventh or eighth aspect of present invention, administration of the fusion protein or the pharmaceutical composition comprising the fusion protein can be according to any available administration scheme that suffices to deliver sufficient active material or active agent into the patient's body. According to one embodiment, administration of the fusion protein or the fusion protein-containing pharmaceutical composition is subcutaneous.

In the context of the different medical uses and methods of treatment of the first, second, fifth, sixth, seventh or eighth aspect of present invention, administration of the DPP-IV inhibitor can be according to any available administration scheme that suffices to deliver sufficient active material or active agent into the patient's body. Depending on the DPP-IV inhibitor used, this can e.g. be perorally, orally, subcutaneously, intramuscularly, pulmonary, by inhalation and/or through sustained release administrations. In one suitable embodiment, the DPP-IV inhibitor is administered orally.

In the context of the different medical uses and methods of treatment of the first, second, fifth, sixth, seventh or eighth aspect of present invention, administration of the anti-diabetic drug can be according to any available administration scheme that suffices to deliver sufficient active material or active agent into the patient's body. Depending on the the anti-diabetic drug used, this can e.g. be perorally, orally, subcutaneously, intramuscularly, pulmonary, by inhalation and/or through sustained release administrations. In one suitable embodiment, the anti-diabetic drug is administered orally.

In a ninth aspect, present invention concerns a nucleic acid encoding the fusion protein of present invention, preferably comprising or consisting of one of the following nucleic acid sequences:

a) a nucleic acid sequence according to one of the sequences with SEQ ID NOs: 27 to 38

b) a nucleic acid coding for a protein sequence according to SEQ ID NOs: 15 to 26 and 39 to 44

c) a nucleic acid hybridizing under stringent conditions with a nucleic acid according to a) or b).

In a tenth aspect, the present invention concerns a vector comprising the nucleic acid of present invention suitable for expression of the encoded protein in a eukaryotic or prokaryotic host.

A vector is a circular or linear polynucleotide molecule, e.g. a DNA plasmid, bacteriophage or cosmid, by aid of which polynucleotide fragments (e.g. cut out from other vectors or amplified by PCR and inserted in the cloning vector) can specifically be amplified in suitable organisms (i.e. cloning). Suitable organisms are mostly single cell organisms with high proliferation rates, like e.g. bacteria or yeast. Suitable organisms can also be cells isolated and cultivated from multicellular tissues, like e.g. cell lines generated from diverse organisms (e.g. SF9 cells from Spodoptera frugiperda, etc.). Suitable cloning vectors are known in the art and commercially available at diverse biotech suppliers like, e.g. Roche Diagnostics, New England Biolabs, Promega, Stratagene and many more. Suitable cell lines are e.g. commercially available at the American Type Culture Collection (ATCC)

In an eleventh aspect, the present invention concerns a cell stably or transiently carrying the vector of present invention and capable of expressing the fusion protein of present invention under appropriate culture conditions.

The cell can be any prokaryotic or eukaryotic cell capable of being transfected with a nucleic acid vector and of expressing a gene. These comprise principally primary cells and cells from a cell culture, preferably a eukaryotic cell culture comprising cells derived either from multicellular organisms and tissue (such as HeLA, CHO, COS, SF9 or 3T3 cells) or single cell organisms such as yeast (e.g. S. pombe or S. cerevisiae), or a prokaryotic cell culture, preferably Pichia or E.coli. Cells and samples derived from tissue can be gained by well-known techniques, such as taking of blood, tissue punction or surgical techniques.

In a twelfth aspect, the present invention concerns a method of preparing the fusion protein of present invention comprising

a) cultivating a culture of cells of present invention under appropriate culture conditions for the fusion protein to be expressed in the cell, or

b) harvesting or purifying the fusion protein from a culture comprising cells of present invention that have been cultivated under appropriate conditions for the fusion protein to be expressed, or

c) cultivating the cells of present invention according to step a) and purifying the fusion protein according to step b) and optionally

d) cleaving of the His-tag using a protease if the fusion protein is a fusion protein comprising a His-tag.

Methods for practicing the ninth, tenth, eleventh and twelfth aspects of present invention, as well as methods for generation of the proteins according to the first aspect of present invention can be gained from the general description, the Definitions section, the following molecular methods section, the cited literature for standard methods as well as from the Examples section.

Molecular Biological Methods for Cloning and Expression of Proteins

Methods for cloning of nucleic acids and expression of proteins are well known in the art. Some general reference for cloning and generation of the proteins and nucleic acids of the invention will be given in the following, without being meant to be limiting.

The preparation of recombinant polypeptide or polynucleotide molecules and the purification of naturally occurring molecules from cells or tissue, as well as the preparation of cell- or tissue extracts is well known to the person of skill in the art (see e.g. also the standard literature listed below).

These comprise e.g. amplifying polynucleotides of desired length via the polymerase chain reaction (PCR) on the basis of the published genomic or coding polynucleotide sequences and the subsequent cloning of the produced polynucleotides in host cells (see e.g. standard literature listed below).

The PCR is an in vitro technique that enables the specific amplification of sequence stretches having nucleotide stretches of known sequence in their 5′ and 3′ vicinit. For amplifying the sequence of choice, short single-stranded DNA molecules (“primers”) are used, which are complementary to the sequence stretches framing the polynucleotide sequence to be amplified. The polynucleotide template can either be DNA or RNA. By choosing defined sequences of incubation steps at defined temperatures and of defined time intervals, that are repeated periodically, the polynucleotide of interest is amplified exponentially.

Suitable primers can be generated by means of chemical synthesis according to well-known protocols. Such primers are also commercially available by commercial vendors.

DNA and RNA templates, also cDNA templates can be generated by means of well known standard procedures (such as DNA templates cloned by aid of cloning vectors; the preparation of genomic DNA or RNA from culture cells, tissue, etc or preparation of cDNA from such sources of RNA, etc., see, e.g. the below standard literature) and can also be purchased from commercial suppliers, such as Promega and Stratagene, etc. Suitable buffers and enzymes as well as reaction protocols for performing the PCR are known in the art and commercially available as well. The reaction product can be purified be known procedures (e.g. gel purification or column purification).

Another method of generating isolated polynucleotides is the cloning of a desired sequence and its subsequent complete or partial purification by means of standard methods. For generating isolated polypeptides, the polynucleotides are cloned into expression vectors and the polypeptides are expressed in suitable host organisms, preferably single cell organisms like suitable strains of bacteria or yeast, followed by the subsequent complete or partial purification of the polypeptide.

Methods of production of isolated nucleic acid molecules are well known in the art. These comprise e.g. amplifying polynucleotides of desired length via the polymerase chain reaction (PCR) on the basis of the published genomic or coding polynucleotide sequences and the subsequent cloning of the produced polynucleotides in host cells.

PCR (polymerase chain reaction) is an in vitro technique that enables the specific amplification of sequence stretches having nucleotide stretches of known sequence in their 5′ and 3′ vicinity. In order to amplify a given sequence, it is sufficient, if the sequence in the 5′ region of the sequence to be amplified is known. In this case, a fragment of the polynucleotide to be amplified is to be generated first (this can be done by known techniques, such as digestion with a restriction endonuclease). Next, a DNA-molecule of known sequence (a “linker”) is coupled to the 3′-end of the generated polynucleotide fragment by means of a ligase (such as T4 DNA ligase, which is commercially available from different suppliers). The resulting sequence is thus surrounded by two known sequences, the known 5′-sequence and 3′ the known linker sequence, enabling the specific amplification by PCR (in this case a linker-mediated PCR “ImPCR”).

For amplifying the sequence of choice, short single-stranded DNA molecules (“primers”) are used, which are complementary to the sequence stretches framing the polynucleotide sequence to be amplified. The polynucleotide template can either be DNA or RNA. The primers are then annealed to the single stranded template and elongated, under defined and well known conditions, by specific enzymes, the so called polymerases (either DNA polymerases recognising DNA as template and producing complementary DNA polynucleotides or reverse transcriptases, recognising RNA as template and producing complementary DNA polynucleotides), thus leading to the generation of new DNA strands having a sequence complementary to that of the template strand. By chosing defined sequences of incubation steps at defined temperatures and of defined time intervalls, that are repeated periodically, a sequence of denaturation/annealing/polymerisation steps is generated that ultimately leads to the exponential amplification of the polynucleotide of interest. In order to be able to apply the necessary temperatures for denaturation without destroying the polymerase, heat-stable enzymes, well tolerating temperatures as high as 95° C. and more, such as Taq-DNA polymerase (DNA polymerase from thermus aquaticus), PFU etc, both commercially available from different suppliers, are used. The choice of suitable polymerases depends on the purpose of use (e.g. for cloning by PCR, polymerases with proofreading capabilities, such as PFU are preferably chosen) and belongs to the skills of the person of the art.

A typical PCR reaction comprises the polynucleotide template (e.g. 0,01 to 20 ng), two suitable primers (in a concentration of e.g. 0,2 to 2 μM each), dNTPs (in a concentration of e.g. 200 μM each), 1 to 2mM MgCl2 and 1 to 10 units of a heat-stable polymerase, such as Taq. Typical components and buffers are well known to the person of skill in the art and commonly available by commercial suppliers.

Suitable primers can be generated by means of chemical synthesis according to well known protocols. Such primers are also commercially available by different commercial vendors.

DNA and RNA templates, also cDNA templates can be generated by means of well known standard procedures (see, e.g. the below standard literature) and can also be purchased from commercial suppliers, such as Promega and Stratagene, etc. Suitable buffers and enzymes for performing the PCR are known in the art and commercially available as well.

By means of specific vectors well known in the art, isolated polypeptides, e.g. the fusion proteins according to present invention can be produced using the subcloned polynucleotides. This is preferably performed by expression in suitable host cells, e.g. bacteria (preferably E. coli strains) or eucaryotic hosts (e.g. SF9 cells, yeast cells, etc.). To this end, the polynucleotide is subcloned in an expression vector suitable for the type of host cell chosen and subsequently introduced into the host cell of choice. Suitable methods for transformation and transfection are well known in the art as well as conditions for cell cultivation and induction of heterologous protein expression (see e.g. standard literature listed below).

Literature for Standard Laboratory Methods

If not indicated otherwise, standard laboratory methods were or can be performed according to the following standard literature:

Sambrook et al. (1989) Molecular Cloning: A Laboratory Manual. Second edition. Cold Spring Harbor Laboratory Press, Cold Spring Harbor, N.Y. 545 pp;

Current Protocols in Molecular Biology; regularly updated, e.g. Volume 2000; Wiley & Sons, Inc; Editors: Fred M. Ausubel, Roger Brent, Robert Eg. Kingston, David D. Moore, J. G. Seidman, John A. Smith, Kevin Struhl.

Current Protocols in Human Genetics; regularly uptdated; Wiley & Sons, Inc; Editors: Nicholas C. Dracopoli, Honathan L. Haines, Bruce R. Korf, Cynthia C. Morton, Christine E. Seidman, J. G. Seigman, Douglas R. Smith.

Current Protocols in Protein Science; regularly updated; Wiley & Sons, Inc; Editors: John E. Coligan, Ben M. Dunn, Hidde L. Ploegh, David W. Speicher, Paul T. Wingfield.

Molecular Biology of the Cell; third edition; Alberts, B., Bray, D., Lewis, J., Raff, M., Roberts, K., Watson, J. D.; Garland Publishing, Inc. New York & London, 1994;

Short Protocols in Molecular Biology, 5th edition, by Frederick M. Ansubel (Editor), Roger Brent (Editor), Robert E. Kingston (Editor), David D. Moore (Editor), J. G. Seidman (Editor), John A. Smith (Editor), Kevin Struhl (Editor), October 2002, John Wiley & Sons, Inc., New York”

Transgenic Animal Technology A Laboratory Handboook. C. A. Pinkert, editor; Academic Press Inc., San Diego, Calif., 1994 (ISBN: 0125571658)

Gene targeting: A Practical Approach, 2^{nd} Ed., Joyner A L, ed. 2000. IRL Press at Oxford University Press, New York;

Manipulating the Mouse Embryo: A Laboratory Manual. Nagy, A, Gertsenstein, M., Vintersten, K., Behringer, R., 2003, Cold Spring Harbor Press, New York;

Remington's Pharmaceutical Sciences, 17^{th} Edition, 1985 (for physiologically tolerable salts (anorganic or organic), see esp. p. 1418)

Aguilar H N, Zielnik B, Tracey C N, Mitchell B F (2010) Quantification of Rapid Myosin Regulatory Light Chain Phosphorylation Using High-Throughput In-Cell Western Assays: Comparison to Western Immunoblots. PLoS ONE 5(4): e9965. doi: 10.1371/journal.pone.0009965

Preferred Aspects

In the following, preferred aspects of present invention are listed.

1. A fusion protein comprising the polypeptide with structure A-B-C or C-B-A or B-A-C or B-C-A or A-C-B or C-A-B or A-B-C-B-C or A-C-B or A-B-C-B or A-C-B-C, wherein

A is a GLP-1 R (glucagon-like peptide-1 receptor) agonist and

C is an FGF-21 (fibroblast growth factor 21) compound and

B is a Linker comprising about 0, 1 to 1000 amino acids.

2. The fusion protein according to claim 1, wherein the linker comprises a functional moiety conferring one or more additional functions beyond that of linking A and C.

3. The fusion protein according to claim 1 or 2, wherein the linker is a peptide linker.

4. The fusion protein according to one of the claims 1 to 3, wherein the FGF-21 compound is selected from native FGF-21 or an FGF-21 mimetic.

5. The fusion protein according to claim 4, wherein the FGF-21 mimetic is selected from a protein having at least about 96% amino acid sequence identity to the amino acid sequence shown in SEQ ID NO: 3 and having FGF-21 activity, a FGF-21 fusion protein and/or a FGF-21 conjugate.

6. The fusion protein according to claim 4 or 5, wherein the FGF-21 mimetic is selected from a FGF-21 mutein, a FGF-21-Fc fusion protein, a FGF-21-HSA fusion protein and/or a PEGylated FGF-21.

7. The fusion protein according to one of the claims 1-6, wherein the GLP-1R agonist is selected from a bioactive GLP-1, a GLP-1 analogue or a GLP-1 substitute.

8. The fusion protein according to one of the claims 1-7, wherein the GLP-1 R agonist is selected from GLP-1(7-37), GLP-1(7-36)amide, extendin-4, liraglutide, CJC-1131, albugon, albiglutide, exenatide, exenatide-LAR, oxyntomodulin, lixisenatide, geniproside, or a short peptide with GLP-1 R agonistic activity.

9. The fusion protein according to anyone of the claims 1-8, wherein the linker comprises one or more of the following functional moieties a) to g):

a) a moiety conferring increased stability and/or half-life to the fusion such as an XTENylation or PASylation sequence or Elastin-like polypeptides (ELPs);

b) an entry site for covalent modification of the fusion protein such as a cysteine or lysine, residue

c) a moiety with intra- or extracellular targeting function such as a protein-binding scaffold

d) a protease cleavage site such as a FactorXa cleavage site or a cleavage site for another extracellular protease.

e) an albumin binding domain (ABD);

f) a Fc portion of an immunoglobulin, e.g. the Fc portion of IgG4;

g) an amino acid sequence comprising one or more histidine (His linker, abbreviated as “His”) amino acids, for example HAHGHGHAH.

10. The fusion protein according to any one of the claims 1-9, wherein the linker consists of the one or more functional moieties.

11. The fusion protein according to any one of the claims 1-9, wherein the linker comprises additional amino acids in addition to the functional moiety.

12. The fusion protein according to claims 9 to 11, wherein the linker comprises one or more of the following protease cleavage sites:

a) a factor Xa cleavage site and preferably comprising or consisting of the sequence IEGR (SEQ ID NO:11)

b) a protease cleavage site and preferably comprising or consisting of at least one arginine and more preferably comprising or consisting of the sequence GGGRR (SEQ ID NO: 14).

13. The fusion protein according to claims 9 to 12, wherein the linker comprises or consists of an entry site for covalent modification and preferably comprising or consisting of the sequence according to SEQ ID NO:13.

14. The fusion protein according to claims 9 to 13, wherein the linker comprises or consists of a protein stabilisation sequence and preferably comprises a PASylation sequence such as the sequence according toSEQ ID NO: 12.

15. The fusion protein according to claims 9 to 14, wherein the linker comprises or consists of one or more entry sites for covalent modification of the fusion protein such as a cysteine or a lysine and preferably a cysteine.

16. The fusion protein according to claim 15, comprising one or more moieties D being covalently attached to the entry site(s) for covalent modification of the linker.

17. The fusion protein according to claim 16, wherein the covalently attached moiety or moieties D are selected from the list consisting of:

a) a targeting unit such as an antibody or protein-binding scaffold

b) a protein-stabilizing unit such as a hydroxyethyl starch derivative (HES) or a polyethylenglycol or derivative thereof (PEG or PEG derivative)

c) a fatty acid.

18. The fusion protein according one of the claims 1 to 17, comprising a tag for protein-purification such as a His-tag and wherein the tag is preferably N- or C-terminally attached to the fusion protein.

19. The fusion protein according to claim 18 comprising a protease cleavage site between the protein-purification tag and the remaining parts of the fusion protein, wherein the protease cleavage site is preferably a Sumo protease cleavage site.

20. The fusion protein according to any one of the claims 1 to 19, wherein A is an FGF-21 mutein and C is exenatide, exendin-4 or lixisenatide.

21. The fusion protein according to claim 20, wherein B comprises a sequence according toSEQ ID NO:11, SEQ ID NO:12, SEQ ID NO:13 or SEQ ID NO:14.

22. The fusion protein according to claim 20 or 21, wherein A is an FGF-21 mutein comprising or consisting of SEQ ID NO: 2 or 102.

23. The fusion protein according to one of the claims 20 to 22, wherein C is exenatide.

24. The fusion protein according to one of the claims 1 to 23 for use as a medicament.

25. A pharmaceutical composition comprising the fusion protein of any one of the claims 1 to 23 together with a pharmaceutically acceptable excipient.

26. A pharmaceutical composition comprising the fusion protein of any one of the claims 1 to 23 together with a pharmaceutically acceptable excipient for use as a medicament.

27. Article of manufacture comprising

a) the fusion protein according to one of the claims 1 to 23 or the pharmaceutical composition according to one claim 25 and

b) a container or packaging material.

28. A method of treating a disease or disorder of a patient, in which the increase of FGF-21 receptor autophosphorylation or in which the increase of FGF-21 efficacy is beneficial for the curing, prevention or amelioration of the disease or disorder, wherein the method comprises administration to the patient of a fusion protein of any one of the claims 1 to 23 or the pharmaceutical composition of claim 23.

29. A method of treating a cardiovascular disease and/or diabetes mellitus and/or at least one metabolic syndrome which increases the risk of developing a cardiovascular disease and/or diabetes mellitus, preferably Type 2-diabetes in a patient comprising the administration to the patient of a fusion protein of any one of the claims 1 to 23 or the pharmaceutical composition of claim 25.

30. A method of lowering plasma glucose levels, of lowering the lipid content in the liver, of treating hyperlipidemia, of treating hyperglycemia, of increasing the glucose tolerance, of decreasing insulin tolerance, of increasing the body temperature, and/or of reducing weight of a patient comprising the administration to the patient of a fusion protein of any one of the claims 1 to 23 or the pharmaceutical composition of claim 25.

31. A nucleic acid encoding the fusion protein according to any one of the claims 1 to 23, preferably comprising or consisting of one of the following nucleic acid sequences:

a) a nucleic acid sequence according to one of the sequences with ID NOs: 27 to 38

b) a nucleic acid coding for a protein sequence according to SEQ ID NOs: 15 to 26 and 39 to 44

c) a nucleic acid hybridizing under stringent conditions with a nucleic acid according to a) or b).

32. A vector comprising the nucleic acid of claim 31 suitable for expression of the encoded protein in a eucaryotic or procaryotic host.

33. A cell stably or transiently carrying the vector of claim 32 and capable of expressing the fusion protein according to one of the claims 1 to 23 under appropriate culture conditions.

34. A method of preparing the fusion protein of one of the claims 1 to 23 comprising

a) cultivating a culture of cells of claim 33 under appropriate culture conditions for the fusion protein to be expressed in the cell, or

b) harvesting or purifying the fusion protein from a culture comprising cells according to claim 33 that have been cultivated under appropriate conditions for the fusion protein to be expressed, or

c) cultivating the cells according to step a) and purifying the fusion protein according to step b) and optionally

d) cleaving of the His-tag using a protease if the fusion protein is a fusion protein according to one of the claims 18 to 23.

35. The medical use of the fusion protein according to preferred aspect 24, or of the pharmaceutical compound according to preferred aspect 26, wherein the medical use is a use in the treatment of a disease or disorder in which the increase of FGF-21 receptor autophosphorylation or the increase of FGF-21 efficacy is beneficial for the curing, prevention or amelioration of the disease.

36. The medical use of the fusion protein according to preferred aspect 24, or of the pharmaceutical compound according to preferred aspect 26, wherein the medical use is a use in the treatment of a cardiovascular disease and/or diabetes mellitus and/or at least one metabolic syndrome which increases the risk of developing a cardiovascular disease and/or for use in the treatment of diabetes mellitus, preferably Type 2-diabetes.

37. The medical use of the fusion protein according to preferred aspect 24, or of the pharmaceutical compound according to preferred aspect 26, wherein the medical use is a use in the lowering of plasma glucose levels, in the lowering of the lipid content in the liver, for use in treating hyperlipidemia, for use in treating hyperglycemia, for use in increasing the glucose tolerance, for use in decreasing insulin tolerance, for use in increasing the body temperature, and/or for use in reducing weight.

38. The medical use or method of treatment according to any one of the preferred aspects 24, 26, 28 to 30 or 35 to 37 comprising administration of at least one anti-diabetic drug and/or at least one DPP-IV (dipeptidyl peptidase-4) inhibitor.

39. The medical use or method of treatment according to preferred aspect 38, wherein the fusion protein and the anti diabetic drug and/or the DPP-IV inhibitor are administered simultaneously or subsequently.

40. The medical use or method of treatment according to preferred aspect 38 or 39, wherein the anti-diabetic drug is selected from metformin, a thiazolidinedione, a sulphonylurea, and/or insulin.

41. The medical use or method of treatment according to one of the preferred aspects 38 to 40, wherein the DPP-IV inhibitor is selected from sitagliptin, vildagliptin, saxagliptin, linagliptin, adogliptin and/or berberine.

42. The medical use or method of treatment according to one of the preferred aspects 38 to 40, wherein the fusion protein and the DPP-IV inhibitor are combined in one formulation or contained in several formulations.

43. The medical use or method of treatment according to one of the preferred aspects 38 to 40, wherein the fusion protein and the anti diabetic drug(s) are combined in one formulation or contained in several formulations.

44. The medical use or method of treatment according to one of the preferred aspects 38 to 40, wherein the DPP-IV inhibitor and the anti-diabetic drug(s) are combined in one formulation.

45. The medical use or method of treatment according to one of the preferred aspects 38 to 40, wherein the fusion protein and the anti-diabetic drug(s) and/or the othe DPP-IV inhibitor are suitable for simultaneous or subsequent administration(s).

46. The medical use or method of preferred aspect 45, wherein the fusion protein is administered to the patient at the same time as the anti-diabetic drug or the DPP-IV inhibitor or both.

47. The medical use or method of preferred aspect 45, wherein the fusion protein is administered to the patient before or after the anti-diabetic drug or the DPP-IV inhibitor or both.

48. The medical use or method of any one the preferred aspects 36 to 48, wherein the metabolic syndrome is selected from the group consisting of dyslipidemia, fatty liver disease (FLD), dysglycemia, impaired glucose tolerance (IGT), obesity, adipositas, and Type 2-diabetes.

49. The method of any one of the preferred aspects 36 to 47, wherein the cardiovascular disease is atherosclerosis.

50. The medical use or method of any one of the preferred aspects 35 to 51, wherein the patient is selected from the group consisting of: a Type 1-diabetic patient, a Type 2-diabetic patient, a diet-treated Type 2-diabetic patient, a sulfonylurea-treated Type 2-diabetic patient, a far-advanced stage Type 2-diabetic patient, and a long-term insulin-treated Type 2-diabetic patient.

51. The medical use or method of any one of the preferred aspects 35 to 50, wherein the plasma glucose level are lowered, the lipid content in the liver is lowered, the glucose tolerance is increased, the insulin tolerance is increased, the body temperature is increased, and/or the weight is reduced in a diabetic patient, preferably selected from the group consisting of a Type 1-diabetic patient, a Type 2-diabetic patient, in particular a diet-treated Type 2-diabetic patient, a sulfonylurea-treated Type 2-diabetic patient, a far-advanced stage Type 2-diabetic patient and/or a long-term insulin-treated Type 2-diabetic patient.

52. The medical use or method of any one of the preferred aspects 35 to 51, wherein the patient is a mammal, preferably a human being.

53. The medical use or method of any one of the preferred aspects 35 to 52, wherein a therapeutically effective amount of the fusion protein or pharmaceutical composition and optionally the anti-diabetic drug or the DPP IV-inhibitor or both is administered.

54. The medical use or method of any one of the preferred aspects 35 to 53, wherein the fusion protein or the pharmaceutical composition comprising the fusion protein is administered subcutaneously.

55. The medical use or method of any one of the preferred aspects 35 to 54, wherein the DPP-IV inhibitor is administered orally, subcutaneously, intramuscularly, pulmonary, by inhalation and/or through sustained release administrations, preferably, the DPP-IV inhibitor is administered orally.

56. The medical use or method of any one of the preferred aspects 35 to 55, wherein the anti-diabetic drug is administered orally, subcutaneously, intramuscularly, pulmonary, by inhalation and/or through sustained release administrations, preferably, anti-diabetic drug is administered orally.

57. Article of manufacture according to preferred aspect 27 further comprising

c) a pharmaceutical composition comprising a DPP-IV inhibitor and/or

d) a pharmaceutical composition comprising an anti-diabetic drug.

58. Article of manufacture according to preferred aspect 27 or 57 further comprising a data carrier, preferably a label or packaging insert or both containing information concerning one or more of the following:
a) Reference to a medical use or method of treatment according to any one of the preferred aspects 24, 28-30 or 35 to 56,
b) Information concerning storage conditions of the article of manufacture and/or the components thereof
c) Lot or batch number of one or more of the active ingredients such as the fusion protein, the DPP-IV inhibitor or the anti-diabetic drug and/or of the article of manufacture
d) Composition of the article of manufacture and optionally the components thereof
e) Handling instructions of the article of manufacture and optionally its components
f) Expiry date or sell-by date.

59. Article of manufacture according to any one of the preferred aspects 27, 57 or 58 further comprising a device for application of the fusion protein or the pharmaceutical composition comprising the fusion protein and and instructions for use of the device.

60. Article of manufacture according to any one of the preferred aspects 27 or 57 to 59, comprising one or more of the following components a) to e):
a) one or more unit dosage forms comprising the fusion protein
b) one or more unit dosage forms comprising the anti-diabetic drug
c) one or more unit dosage forms comprising the DPP-IV inhibitor
d) a data carrier, the data carrier preferably comprising a label or package insert;
e) a device for application of the fusion protein such as a syringe and instructions for use of the device.

61. Article of manufacture according to preferred aspect 60 comprising one or more unit dosage forms comprising the fusion protein as dry formulation for dissolution in a hermetically sealed container such as a vial, an ampoule or sachette.

62. Article of manufacture according to preferred aspect 61 comprising one or more unit dosage forms comprising the fusion protein as liquid formulation in a hermetically sealed container such as a vial, a sachette, a pre-filled syringe, a pre-filled autoinjector or a cartridge for a reusable syringe or applicator.

63. Article of manufacture according to one of the preferred aspects 60 to 62, comprising one or more unit dosage forms of the anti-diabetic drug as tablet or capsule or other formulation for oral administration in a hermetically sealed container or blister.

64. Article of manufacture according to one of the preferred aspects 60 to 63, comprising one or more unit dosage forms of the DPP-IV inhibitor as tablet or capsule or other formulation for oral administration in a hermetically sealed container or blister

65. Article of manufacture according to any one of the preferred aspects 60 to 64, wherein the quantity of active ingredient is indicated on the hermetically-sealed container or blister.

66. Article of manufacture according to one of the preferred aspects 60 to 65 comprising sufficient unit dosage forms of the fusion protein and preferably also of the anti-diabetic drug or DPP IV-inhibitor or sufficient unit dosage forms of the fusion protein and anti-diabetic drug and DPP IV-inhibitor, for one single, for a two-week (i.e. 14-day) treatment, for a four week (i.e, 28-day) treatment or for a one-month treatment with fusion protein and preferably the anti-diabetic drug or DPP IV-inhibitor or with fusion protein and the anti-diabetic drug and the DPP IV-inhibitor.

67. Article of manufacture according to preferred aspect 66, comprising sufficient unit dosage forms of the fusion protein and optionally for the anti-diabetic drug or the DPP-IV inhibitor or both for a daily administration regime.

68. Article of manufacture according to any one of the preferred aspects 60 to 67, wherein the device is a syringe or another type of injection device.

69. Article of manufacture according to preferred aspect 68, wherein the syringe or injection device is, pre-filled or suitable for subcutaneous injection or both.

In the following, further preferred aspects of present invention are listed.

1. A fusion protein comprising the polypeptide with structure A-B-C or C-B-A or B-A-C or B-C-A or A-C-B or C-A-B or A-B-C-B-C or A-C-B or A-B-C-B or A-C-B-C, wherein

A is a GLP-1 R (glucagon-like peptide-1 receptor) agonist and

C is an FGF-21 (fibroblast growth factor 21) compound and

B is a linker comprising about 0 to 1000 amino acids.

2. The fusion protein according to claim 1, wherein the linker comprises a functional moiety conferring one or more additional functions beyond that of linking A and C.

3. The fusion protein according to claim 1 or 2, wherein the linker is a peptide linker.

4. The fusion protein according to one of the claims 1 to 3, wherein the FGF-21 compound is selected from the group of native FGF-21, FGF-21 mimetic or SEQ ID NO: 3.

5. The fusion protein according to claim 4, wherein the FGF-21 mimetic is selected from a protein having at least about 80% amino acid sequence identity to the amino acid sequence shown in SEQ ID NO: 3 and having FGF-21 activity, a FGF-21 fusion protein and/or a FGF-21 conjugate

6. The fusion protein according to claim 4, wherein the FGF-21 mimetic is selected from a protein having at least about 90% amino acid sequence identity to the amino acid sequence shown in SEQ ID NO: 3 and having FGF-21 activity, a FGF-21 fusion protein and/or a FGF-21 conjugate

7. The fusion protein according to claim 4, wherein the FGF-21 mimetic is selected from a protein having at least about 96% amino acid sequence identity to the amino acid sequence shown in SEQ ID NO: 3 and having FGF-21 activity, a FGF-21 fusion protein and/or a FGF-21 conjugate.

8. The fusion protein according to any of claims 4 -7, wherein the FGF-21 mimetic is selected from a FGF-21 mutein, a FGF-21-Fc fusion protein, a FGF-21-HSA fusion protein and/or a PEGylated FGF-21.

9. The fusion protein according to one of the claims 1-8, wherein the GLP-1R agonist is selected from a bioactive GLP-1, a GLP-1 analogue or a GLP-1 substitute.

10. The fusion protein according to one of the claims 1-9, wherein the GLP-1R agonist is selected from GLP-1(7-37), GLP-1(7-36)amide, exendin-4, liraglutide, CJC-1131, albugon, albiglutide, exenatide, exenatide-LAR, oxyntomodulin, lixisenatide, geniproside, or a short peptide with GLP-1 R agonistic activity.

11. The fusion protein according to anyone of the claims 1-10, wherein the linker comprises one or more of the following functional moieties a) to h):

a) a moiety conferring increased stability and/or half-life to the fusion such as an XTENylation or PASylation sequence or Elastin-like polypeptides (ELPs);

b) an entry site for covalent modification of the fusion protein such as a cysteine or lysine residue

c) a moiety with intra- or extracellular targeting function such as a protein-binding scaffold

d) a protease cleavage site such as a FactorXa cleavage site or a cleavage site for another extracellular protease;

e) a Fc portion of an immunoglobulin, e.g. the Fc portion of IgG4;

f) HSA;

g) an amino acid sequence comprising one or more histidine (His linker, abbreviated as “His” or “His tag”) amino acids, for example HAHGHGHAH.

h) an albumin binding domain (ABD).

12. The fusion protein according to any one of the claims 1-11, wherein the linker consists of the one or more functional moieties.

13. The fusion protein according to any one of the claims 1-10, wherein the linker comprises additional amino acids in addition to the functional moiety.

14. The fusion protein according to claims 11 to 13, wherein the linker comprises one or more of the following protease cleavage sites:

a) a factor Xa cleavage site and preferably comprising or consisting of the sequence IEGR (SEQ ID NO:11)

b) a protease cleavage site and preferably comprising or consisting of at least one arginine and more preferably comprising or consisting of the sequence GGGRR (SEQ ID NO: 14).

15. The fusion protein according to claims 11 to 14, wherein the linker comprises or consists of an entry site for covalent modification and preferably comprising or consisting of the sequence according to SEQ ID NO:13.

16. The fusion protein according to claims 11 to 15, wherein the linker comprises or consists of a protein stabilisation sequence and preferably comprises a PASylation sequence selected from the group of: SEQ ID NO:12, SEQ ID NO: 95, SEQ ID NO: 96, SEQ ID NO: 97, SEQ ID NO: 98, SEQ ID NO: 99, SEQ ID NO: 100, and SEQ ID NO: 101.

17. The fusion protein according to claims 11 to 16, wherein the linker comprises or consists of one or more entry sites for covalent modification of the fusion protein such as a cysteine or a lysine and preferably a cysteine.

18. The fusion protein according to claim 17, comprising one or more moieties D being covalently attached to the entry site(s) for covalent modification of the linker.

19. The fusion protein according to claim 18, wherein the covalently attached moiety or moieties D are selected from the list consisting of:

a) a targeting unit such as an antibody or protein-binding scaffold

b) a protein-stabilizing unit such as a hydroxyethyl starch derivative (HES) or a polyethylenglycol or derivative thereof (PEG or PEG derivative)

c) a fatty acid.

20. The fusion protein according one of the claims 1 to 19, comprising a tag for protein-purification such as a His-tag and wherein the tag is preferably N- or C-terminally attached to the fusion protein.

21. The fusion protein according to claim 20 comprising a protease cleavage site between the protein-purification tag and the remaining parts of the fusion protein, wherein the protease cleavage site is preferably a Sumo protease cleavage site.

22. The fusion protein according to any one of the claims 1 to 21, wherein A is an FGF-21 mutein and C is exenatide, exendin-4 or lixisenatide.

23. The fusion protein according to claim 22, wherein B has a sequence selected from the group of: SEQ ID NO:11, SEQ ID NO:12, SEQ ID NO:13,SEQ ID NO:14, SEQ ID NO: 95, SEQ ID NO: 96, SEQ ID NO: 97, SEQ ID NO: 98, SEQ ID NO: 99, SEQ ID NO: 100, and SEQ ID NO: 101.

24. The fusion protein according to claim 22 or 23, wherein A is an FGF-21 mutein comprising or consisting of SEQ ID NO: 102.

25. The fusion protein according to one of the claims 22 to 24, wherein C is exenatide.

26. The fusion protein according to one of the claims 1 to 25 for use as a medicament.

27. A pharmaceutical composition comprising the fusion protein of any one of the claims 1 to 25 together with a pharmaceutically acceptable excipient.

28. A pharmaceutical composition comprising the fusion protein of any one of the claims 1 to 25 together with a pharmaceutically acceptable excipient for use as a medicament.

29. Article of manufacture comprising

a) the fusion protein according to one of the claims 1 to 25 or the pharmaceutical composition according to one claim 27 and

b) a container or packaging material.

30. A method of treating a disease or disorder of a patient, in which the increase of FGF-21 receptor autophosphorylation or in which the increase of FGF-21 efficacy is beneficial for the curing, prevention or amelioration of the disease or disorder, wherein the method comprises administration to the patient of a fusion protein of any one of the claims 1 to 25 or the pharmaceutical composition of claim 25.

31. A method of treating a cardiovascular disease and/or diabetes mellitus and/or at least one metabolic syndrome which increases the risk of developing a cardiovascular disease and/or diabetes mellitus, preferably Type 2-diabetes in a patient comprising the administration to the patient of a fusion protein of any one of the claims 1 to 25 or the pharmaceutical composition of claim 27.

32. A method of lowering plasma glucose levels, of lowering the lipid content in the liver, of treating hyperlipidemia, of treating hyperglycemia, of increasing the glucose tolerance, of decreasing insulin tolerance, of increasing the body temperature, and/or of reducing weight of a patient comprising the administration to the patient of a fusion protein of any one of the claims 1 to 25 or the pharmaceutical composition of claim 27.

33. A nucleic acid encoding the fusion protein according to any one of the claims 1 to 25, preferably comprising or consisting of one of the following nucleic acid sequences:

a) a nucleic acid sequence according to one of the sequences with ID NOs: 27 to 38

b) a nucleic acid coding for a protein sequence according to SEQ ID NOs: 15 to 26 and 39 to 44

c) a nucleic acid hybridizing under stringent conditions with a nucleic acid according to a) or b).

34. A vector comprising the nucleic acid of claim 33 suitable for expression of the encoded protein in a eukaryotic or prokaryotic host.

35. A cell stably or transiently carrying the vector of claim 34 and capable of expressing the fusion protein according to one of the claims 1 to 25 under appropriate culture conditions.

36. A method of preparing the fusion protein of one of the claims 1 to 25 comprising

a) cultivating a culture of cells of claim 35 under appropriate culture conditions for the fusion protein to be expressed in the cell, or

b) harvesting or purifying the fusion protein from a culture comprising cells according to claim 35 that have been cultivated under appropriate conditions for the fusion protein to be expressed, or

c) cultivating the cells according to step a) and purifying the fusion protein according to step b) and optionally

d) cleaving of the His-tag using a protease if the fusion protein is a fusion protein according to one of the claims 20 to 25.

One further preferred embodiment of the present invention is a fusion protein having the following structure:

Exenatide-(B1)ₙ-HSA-(B2)ₙ-FGF-21, wherein
B1 is (GₐS_{b})_{c}; and
B2 is (GₓS_{y})_{z},

wherein a, b, c, x, y, z, n=0 1, 2, 3, 4, 5, 6, 7, 8, 9, 10.

One further preferred embodiment of the present invention is a fusion protein having the following structure:

Exenatide-FGF-21-(GGGGS)ₘ-ABD-(GGGGS)ₙ-FGF-21,

wherein m and n=1, 2, 3, 4, 5, 6, 7, 8, 9, 10.

One further preferred embodiment of the present invention is a fusion protein having the following structure:

Exenatide-FGF-21-(GGGGS)ₙ-ABD,

wherein n=1, 2, 3, 4, 5, 6, 7, 8, 9, 10.

One further preferred embodiment of the present invention is a fusion protein having the following structure:

Exenatide-(GGGGS)ₘ-ABD-(GGGGS)ₙ-FGF-21,

wherein m and n=1, 2, 3, 4, 5, 6, 7, 8, 9, 10.

The following figures and examples are for the purpose of illustration only and are not intended to be limiting of the present invention.

### BRIEF DESCRIPTION OF THE FIGURES

FIG. 1: Dose dependent in vitro activation of either hGLP-1 R (A), human FGFR1c+KLB (B) or the downstream effector ERK (C).

A) Agonism of compounds for human glucagon-like peptide-1 receptor (GLP-1 R) was determined by functional assays measuring cAMP response of HEK-293 cell line stably expressing human GLP-1 receptor. The cAMP content of the cells was determined using a kit from Cisbio Corp. (cat. no. 62AM4PEC) based on HTRF (Homogenous Time Resolved Fluorescence). EC50 values were obtained from dose-response curves and are summarized in table 1.

B) The FGF induced FGFR autophosphorylation was measured via a specific and highly sensitive In-Cell Western (ICW) in CHO cells stable overexpressing human FGFR1c together with human betaKlotho (KLB). In-Cell Western assay is an immunocytochemical assay usually performed in microplate format. Target-specific primary antibodies and infrared-labelled secondary antibodies are used to detect target proteins in fixed cells, and fluorescent signal from each well is quantified (e.g. the In-Cell Western assay from LI-COR Biosciences, USA).

EC50 values were obtained from dose-response curves and are summarized in table 1.

C) Dose dependent in vitro activation of the downstream effector ERK. Activation of the downstream effector of FGF signaling, the MAP kinase ERK1/2, was determined via In-Cell Western assay in CHO cells stable overexpressing human FGFR1 c and KLB using an antibody directed against the ERK1/2 phosphorylated amino acid residues threonine 202 and tyrosine 204.

EC50 values were obtained from dose-response curves and are summarized in table 1.

FIG. 2: Blood glucose change after 10 days of once-daily subcutaneously treatment in ob/ob mice (A), blood glucose levels during an oral glucose tolerance test (B), and corresponding AUC (C). All data are presented as mean±SEM. Data were analyzed by using one-way ANOVA or two-way ANOVA followed by Dunnett's post test. P values lower than 0.05 were considered significant. *P<0.05, **P<0.01, ***P<0.001 vs. vehicle treated obese control mice.

FIG. 3: a), b), c), d): Sequences of Fusion protein units (a-c: FGF-21 compounds, GLP-1 receptor agonists, functional moieties for constructing the linker), fusion proteins and nucleic acid constructs: FIG. 3 shows FGF-21 compounds, different GLP-1 agonist peptides and linker units for constructing or forming the different modules A, C and B of the fusion proteins.

d) FIG. 3d shows different fusion proteins from N- to C-terminal). Sequence ID numbers 15 to 26 are fusion proteins in the arrangement GLP1 receptor agonist-FGF-21 compound (ABC) comprising different linkers and comprising or not comprising a His tag and Sumo cleavage site. The constructs with HisTag/Sumo cleavage site can be cleaved to constructs excluding the HisTag/Sumo cleavage site leaving only the FGF-21 compound-Linker-GLP1 receptor agonist or the GLP1 receptor agonist-linker-FGF-21 compound fusion protein. Sequence ID Numbers 39 and 40 concern fusion proteins with arrangement FGF-21 compound-GLP1 receptor agonist, (CBA) wherein CR9443 comprises a linker having an intact Factor Xa cleavage site and CR 9444 comprises a GS-rich linker comprising a mutated (defective) Factor Xa cleavage site. Construct 9445 is in the order GLP1 receptor agonist-FGF-21 compound and comprises a defective Factor Xa cleavage site.

e) FIG. 3e shows different nucleic acid sequences of constructs encoding fusion proteins:
SEQ ID NO: 27: Construct CR8829 (not codon optimized) Start —His(6)—SUMO cleavage site—Exenatide—Xa cleavage site—human FGF-21 His29-Ser209—stop
SEQ ID NO: 28 Construct CR8846 (not codon optimized) Start—His(6)—SUMO cleavage site—Exenatide—human FGF-21 His29-Ser209—stop
SEQ ID NO: 29 Construct CR8847 (not codon optimized) Start—His(6)—SUMO cleavage site—Exenatide—GGGRR—human FGF-21 His29-Ser209—stop
SEQ ID NO: 30 Construct CR8848 (not codon optimized) Start—His(6)—SUMO cleavage site—Lixisenatide—human FGF-21 His29-Ser209—stop
SEQ ID NO: 31 Construct CR8849 (not codon optimized) Start—His(6)—SUMO cleavage site—Lixisenatide—Faxtor Xa cleavage site—human FGF-21 His29-Ser209—stop
SEQ ID NO: 32 Construct CR8850 (not codon optimized) Start—His(6)—SUMO cleavage site—Lixisenatide—GGGRR—human FGF-21 His29-Ser209—stop
SEQ ID NO: 33 Construct CR9443 (codon optimized for E.coli) Start—His(6)—SUMO cleavage site—human FGF-21 His29-Ser209—GSGSIEGR—Exenatide—stop
SEQ ID NO: 34 Construct CR9444 (codon optimized for E. coli) Start—His(6)—SUMO cleavage site—human FGF-21 His29-Ser209—GSGSIEGQ—Exenatide—stop
SEQ ID NO: 35 Construct CR9445 (codon optimized for E. coli) Start—His(6)—SUMO cleavage site—Exenatide—IEGQ—human FGF-21 His29-Ser209—stop
SEQ ID NO: 36 Construct CR9446 (codon optimized for E. coli) Start—His(6)—SUMO cleavage site—Exenatide—APASPAS—human FGF-21 His29-Ser209—stop
SEQ ID NO: 37 Construct CR9447 (codon optimized for E. coli) Start—His(6)—SUMO cleavage site—Exenatide—APASCPAS—human FGF-21 His29-Ser209—stop
SEQ ID NO: 38 Construct CR9448 (codon optimized for E.coli) Start—His(6)—SUMO cleavage site—Exenatide—GSGS—human FGF-21 His29-Ser209—stop

FIG. 4: Chemical Structure of Liraglutide.

FIG. 5: Chemical Structure of CJC-1131.

FIG. 6: Body weight development (absolute mean values±SE) of ob/ob mice treated with Exenatide-IEGR-FGF21 fusion protein via Alzet miniosmotic pumps at dosages of 0.03, 0.1, 0.3 and 1 mg/kg.

FIG. 7: Relative body weight change (%, mean±SE) of ob/ob mice treated with Exenatide-IEGR-FGF21 fusion protein via Alzet miniosmotic pumps at dosages of 0.03, 0.1, 0.3 and 1 mg/kg. Treatment of ob/ob mice with the fusion protein Exenatide-IEGR-FGF21 showed a dose dependent decrease of body weight with highest reduction of 17.8% at 1 mg/kg.

FIG. 8: Mean liver weight (g, mean±SE) of ob/ob mice treated with Exenatide-IEGR-FGF21 fusion protein via Alzet miniosmotic pumps at dosages of 0.03, 0.1, 0.3 and 1 mg/kg. Treatment of ob/ob mice with the fusion protein Exenatide-IEGR-FGF21 showed a dose dependent decrease of total liver weight.

FIG. 9: Mean liver triglycerides (mg/g liver weight, mean±SE) of ob/ob mice treated with Exenatide-IEGR-FGF21 fusion protein via Alzet miniosmotic pumps at dosages of 0.03, 0.1, 0.3 and 1 mg/kg. Treatment of ob/ob mice with the fusion protein Exenatide-IEGR-FGF21 showed a dose dependent decrease of liver triglycerides.

FIG. 10: Mean blood glucose concentrations (mmol/l, mean±SE) of ob/ob mice treated with Exenatide-IEGR-FGF21 fusion protein via Alzet miniosmotic pumps at dosages of 0.03, 0.1, 0.3 and 1 mg/kg after 11 days.

FIG. 11: Delta blood glucose values between start and end of the study (mmol/l, mean±SE) at dosages of 0.03, 0.1, 0.3 and 1 mg/kg after 11 days. Treatment of ob/ob mice with the fusion protein Exenatide-IEGR-FGF21 showed a dose dependent decrease of blood glucose after 11 days of chronic infusion.

### EXAMPLES

1. Cloning, Expression and Purification of GLP1-R Agonist/FGF-21 Fusion Proteins

Expression cassette was synthesized by Geneart (Regensburg, Germany) and cloned via Ncol/Xhol or Ncol/BamHl in pET16b vector. Plasmids were transformed in E. coli BL21[DE3] and glycerol stocks were made from fresh transformants. Starting from glycerol stocks recombinants were inoculated in fresh Luria-Bertani (LB) medium+Ampicillin and incubated in a shaking incubator at 37° C. and 150 rpm over night. From this preparatory culture an amount was taken to inoculate fresh LB medium+Amp starting with an OD₆₀₀ of 0.1. When OD₆₀₀ reached 0.6 temperature was decreased to 18° C. and isopropyl-D-thio-galactoside (IPTG) was added to a final concentration of 0.5 mM for the induction of expression. Bacterial cells were collected after 22 hours by centrifugation.

Cells were resuspended in lysis buffer (50 mM Tris pH 8.0, 300 mM NaCl, 1 mM Imidazol, 0.1 mg/ml Lysozym, 2 mM MgCl₂, 25U/ml Benzonase) and lysed by French Press. After centrifugation (4° C., 27000g, 60 min) and filtration with 0.22 μm filter supernatant was put on an IMAC (e.g HisTrap HP) column. Proteins without His-tag were removed using 50 mM Tris pH 8.0, 300 mM NaCl and 40 mM imidazol. SUMO fusion protein was eluted with a step gradient of 250 imidazol. Combined fractions containing the SUMO fusion protein were dialysed against buffer (20 mM Tris pH 8.0, 100 mM NaCl) and cleaved for 24 hours at RT with yeast ULP1 protease in a ratio of 1/250. Cleaved protein was diluted with 50 mM Tris pH 8.5 to decrease sodium chloride to 10 mM. Further purification is done with an anion exchange column (e.g. Source 15Q). His-SUMO tag and other contaminants were removed from target protein using a flat gradient of sodium chloride. Combined fractions containing the target protein were concentrated using disposable ultrafiltration device (e.g. Vivaspin 20, 10 000 MWCO). Final purification step was done by size exclusion chromatography (e.g. Superdex 75) equilibrated with PBS followed by an additional ultrafiltration and steril filtration step.

2. In Vitro Cellular Assay for Human GLP-1 Receptor Efficacy

Agonism of compounds for human glucagon-like peptide-1 (GLP-1) receptor was determined by functional assays measuring cAMP response of HEK-293 cell line stably expressing human GLP-1 receptor.

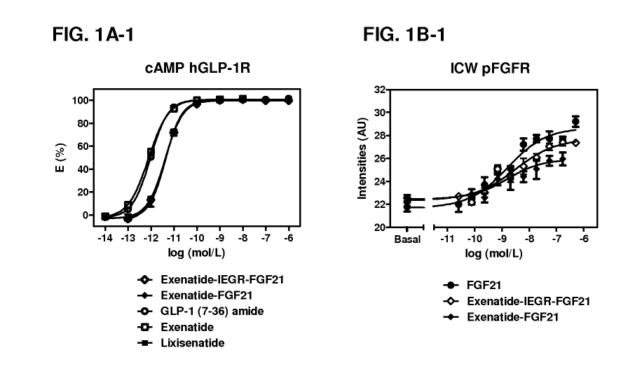

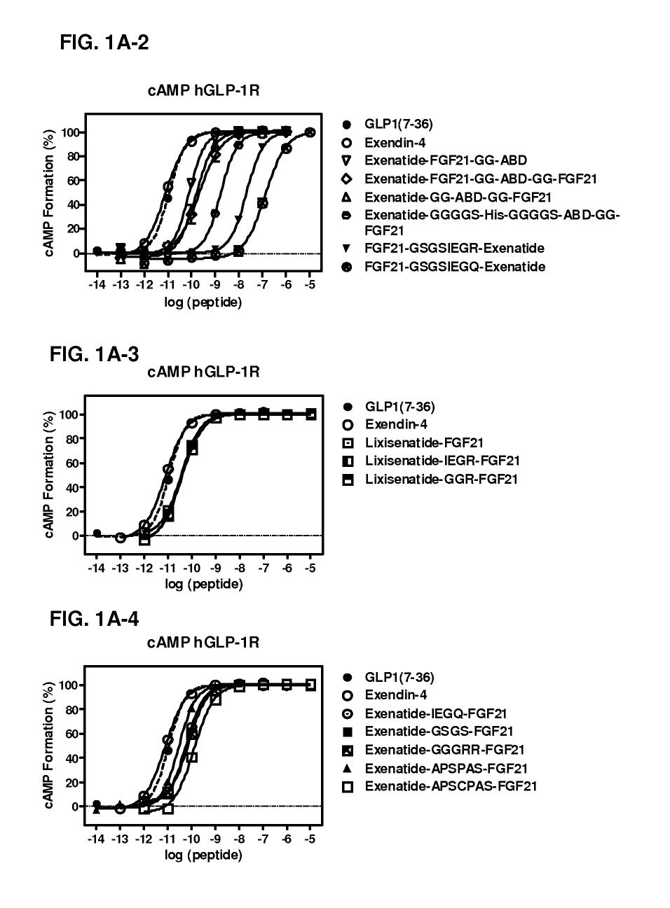

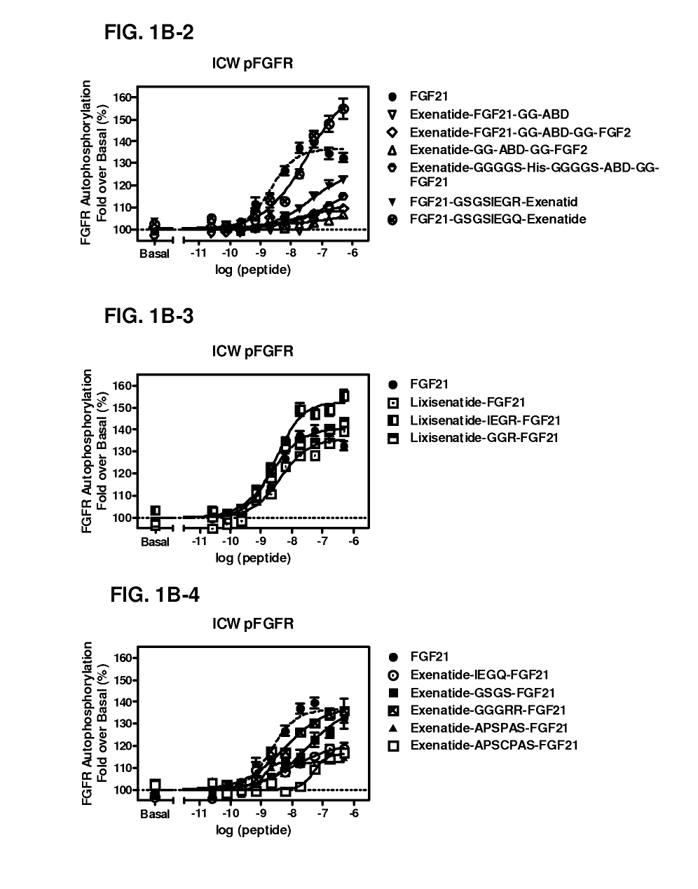

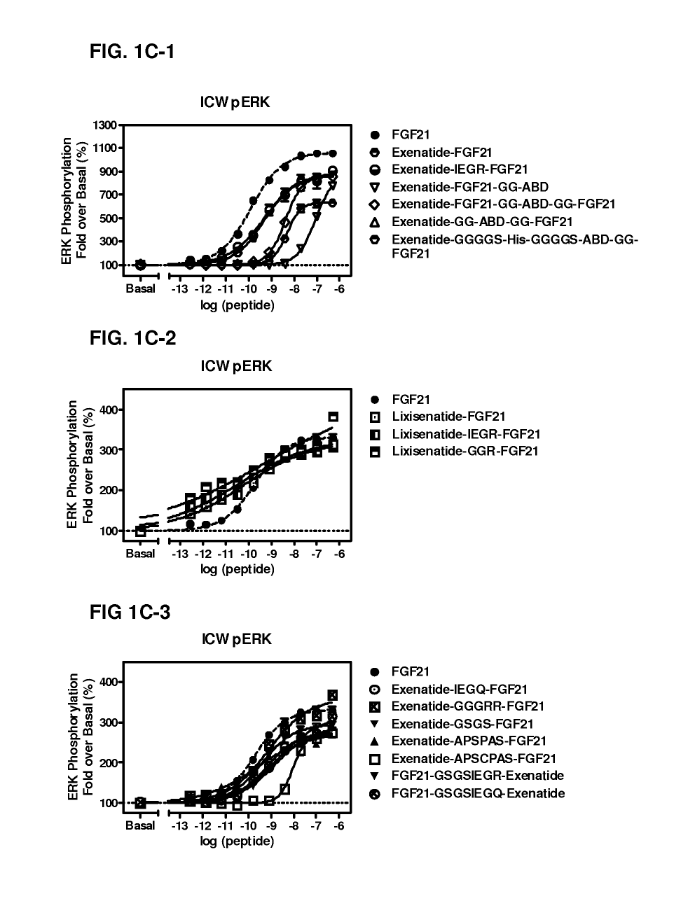

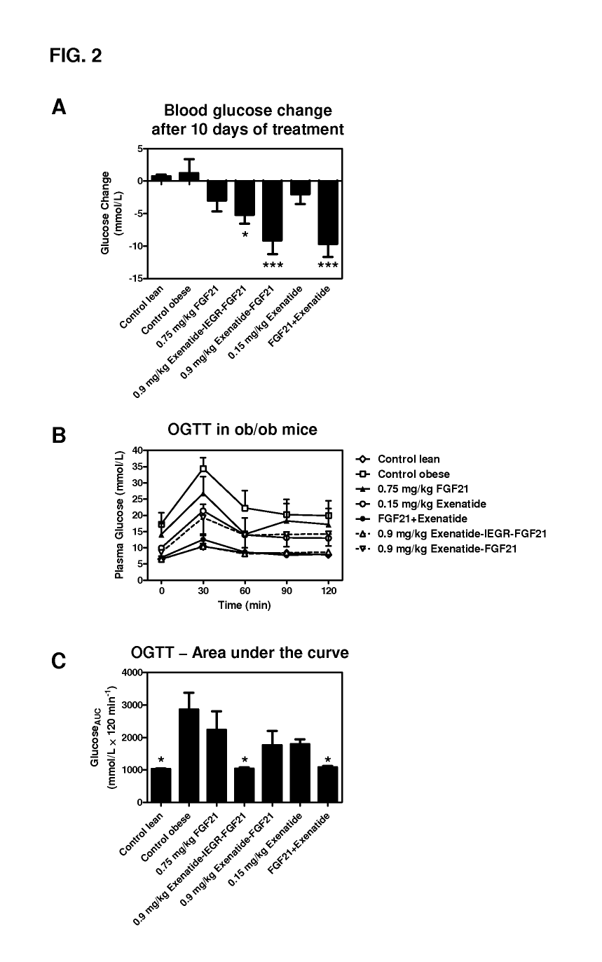

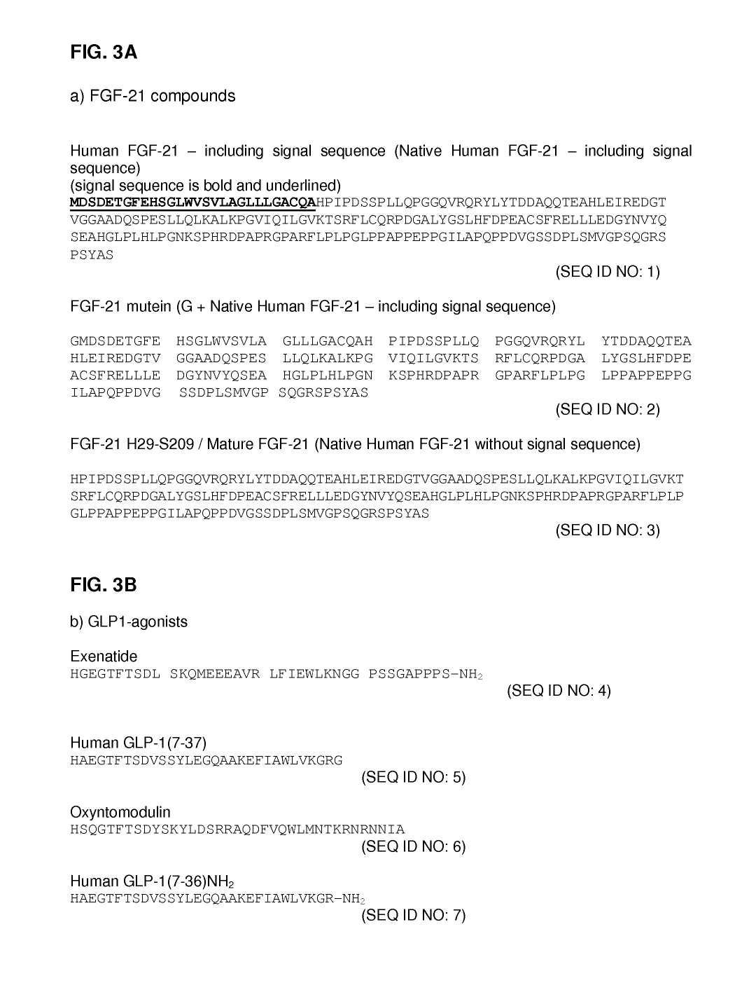

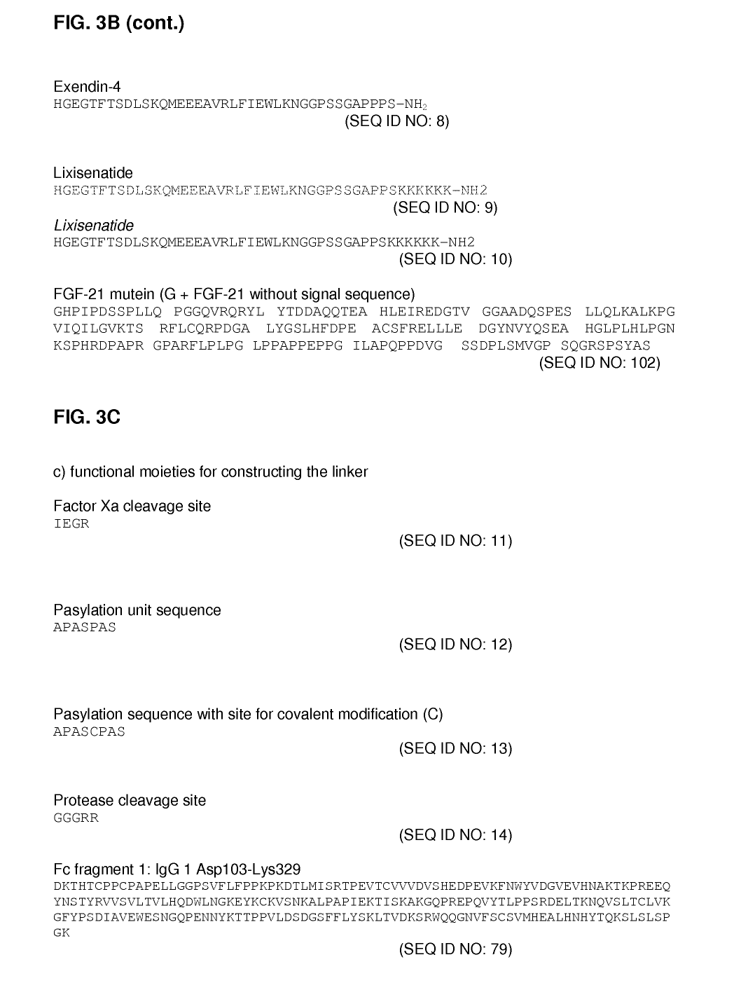

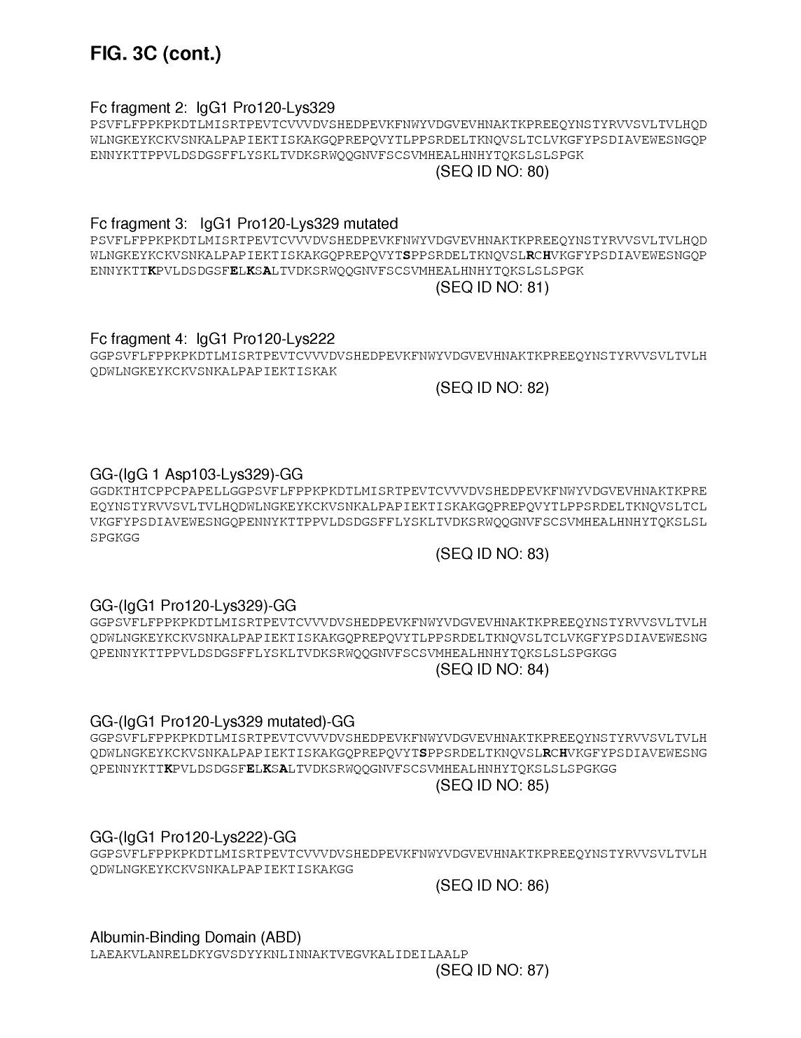

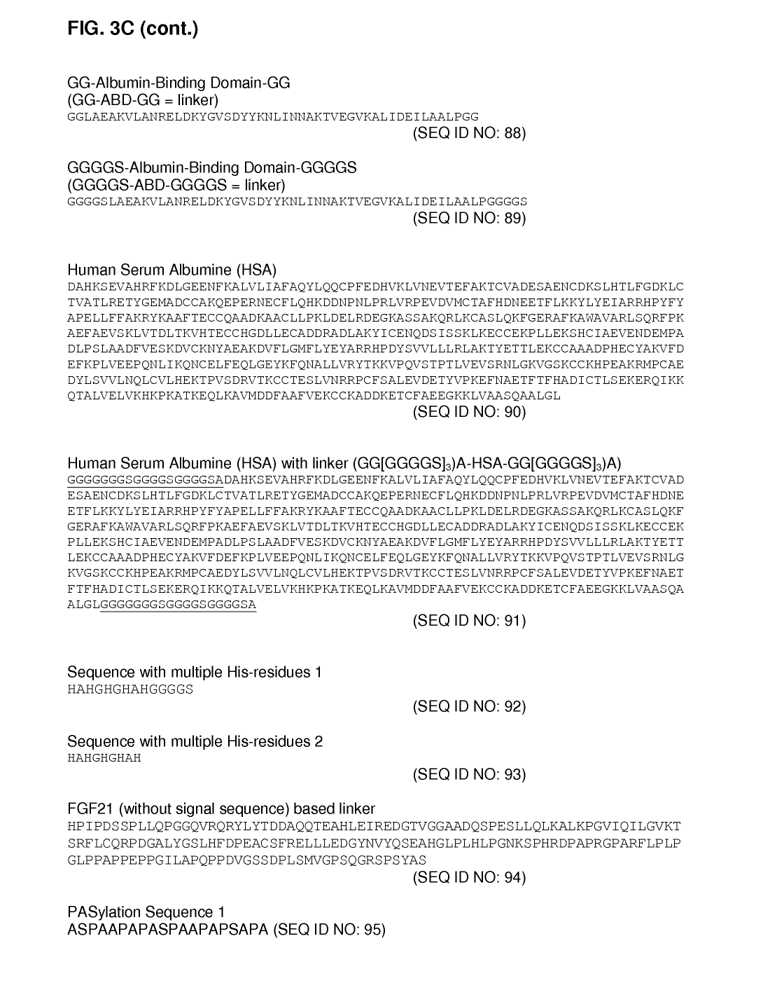

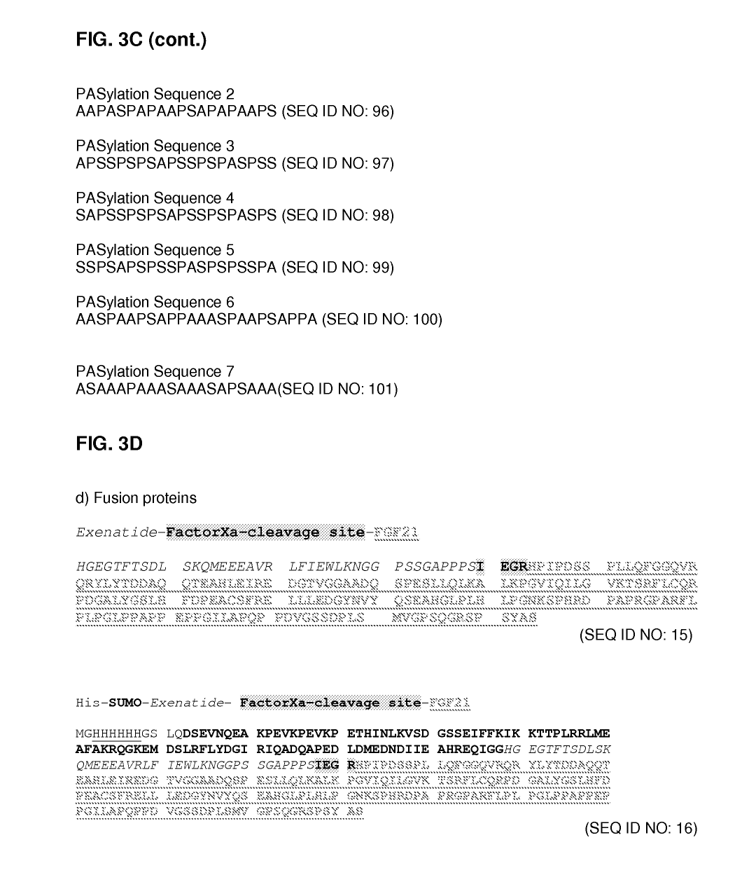

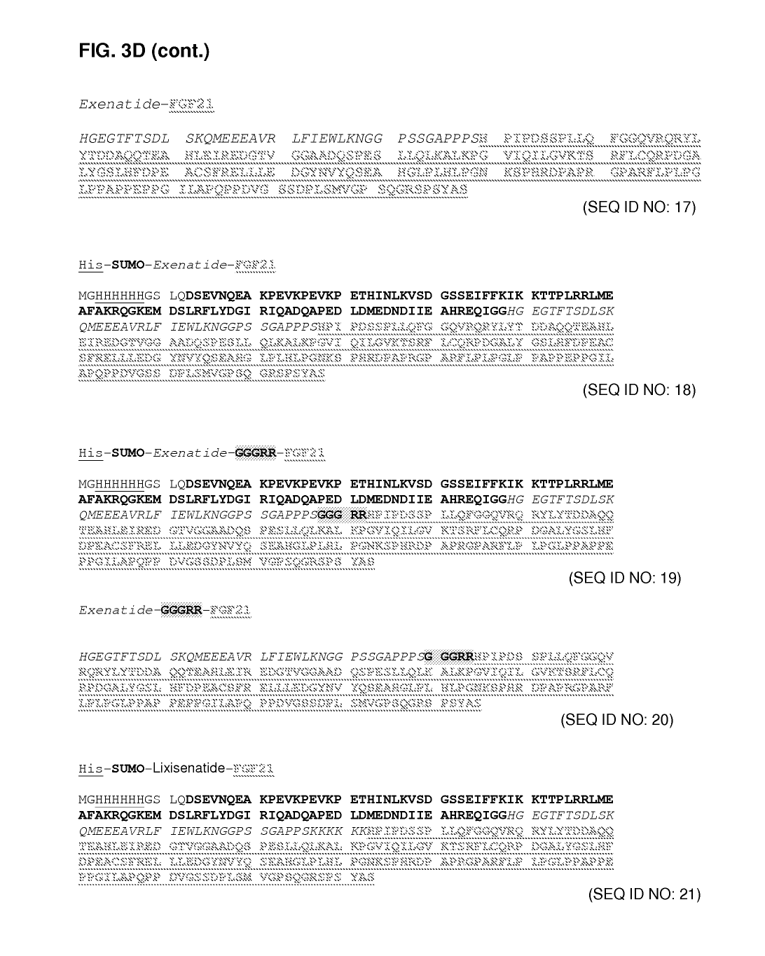

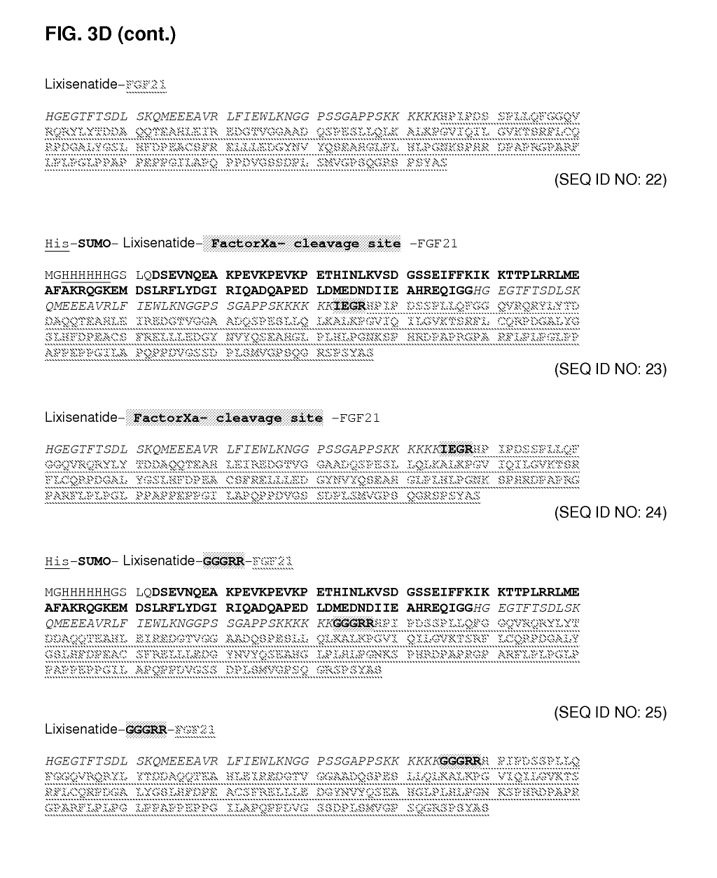

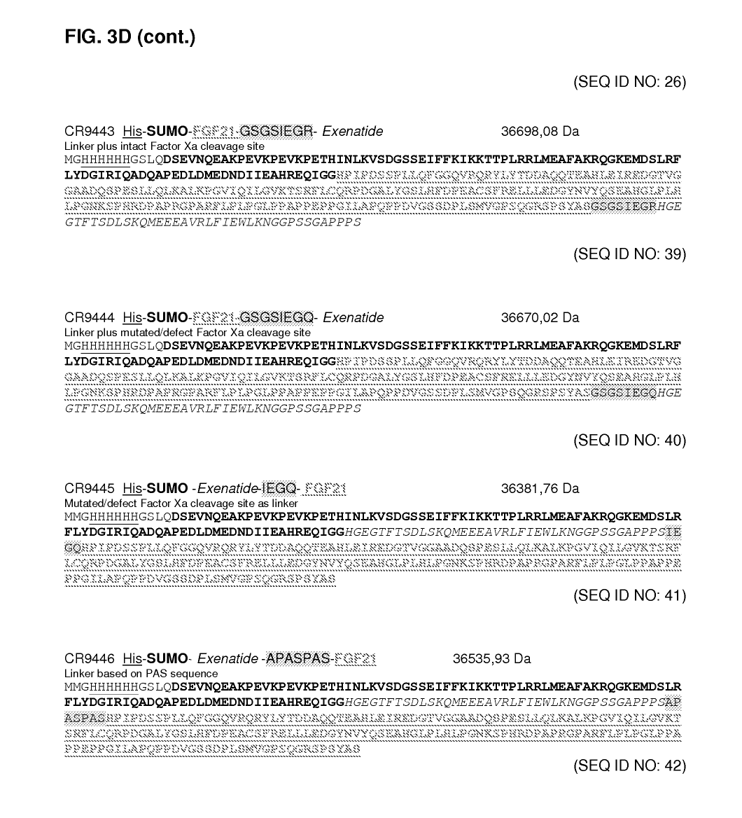

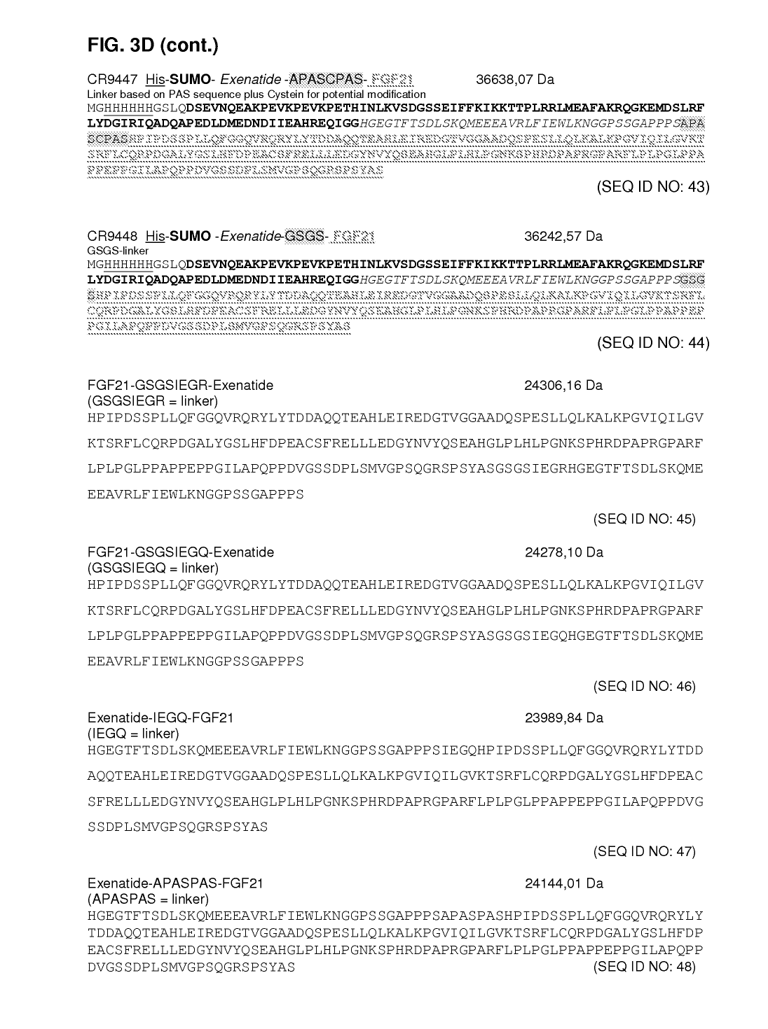

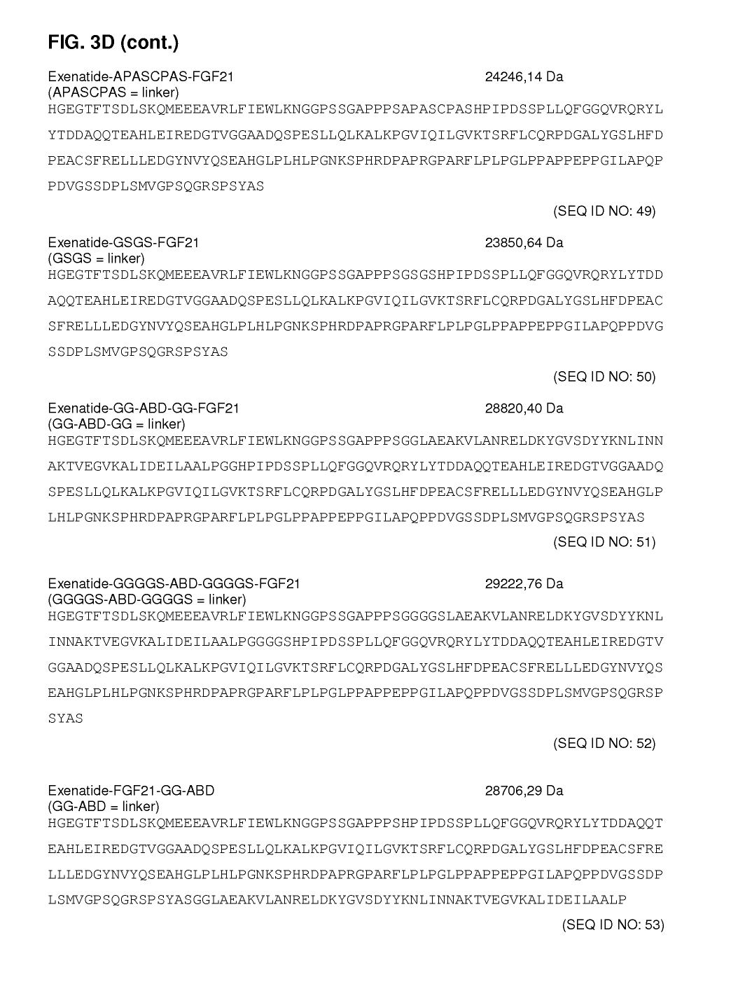

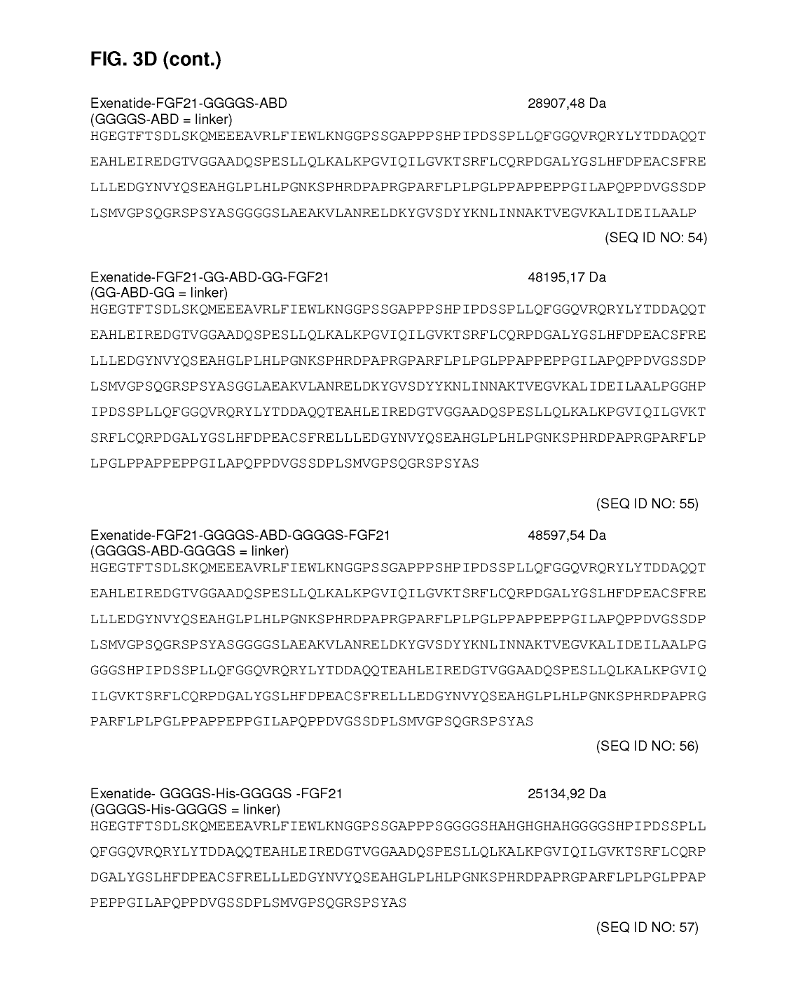

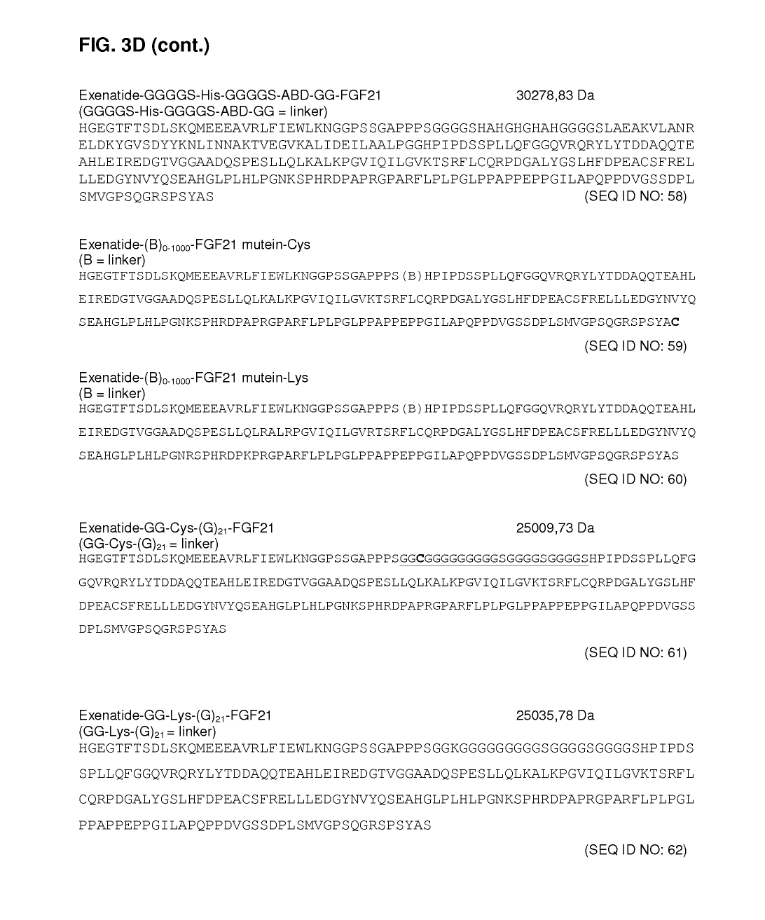

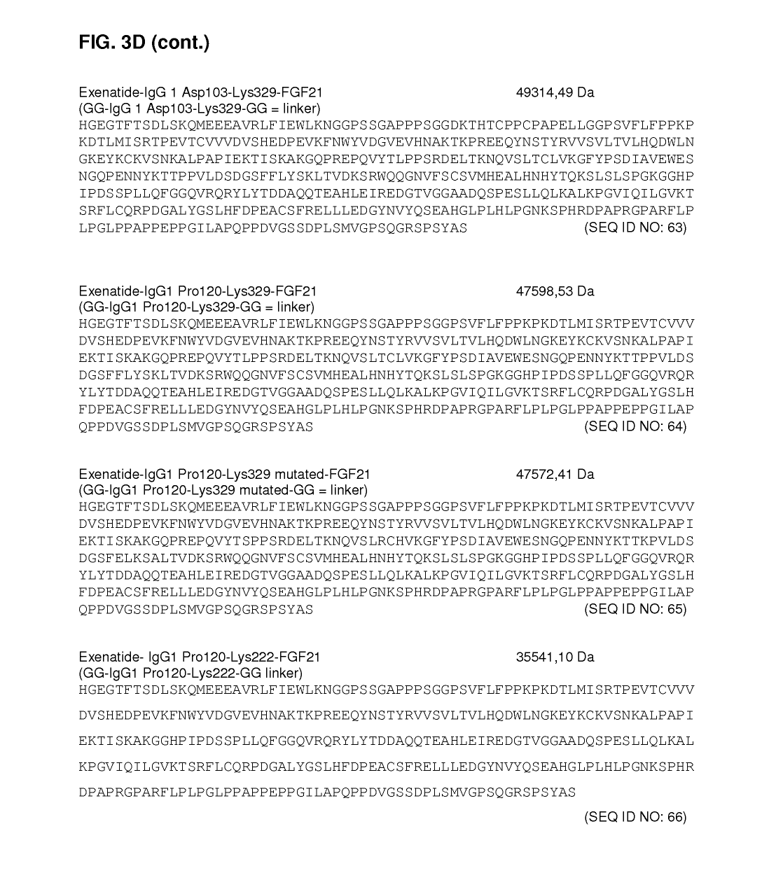

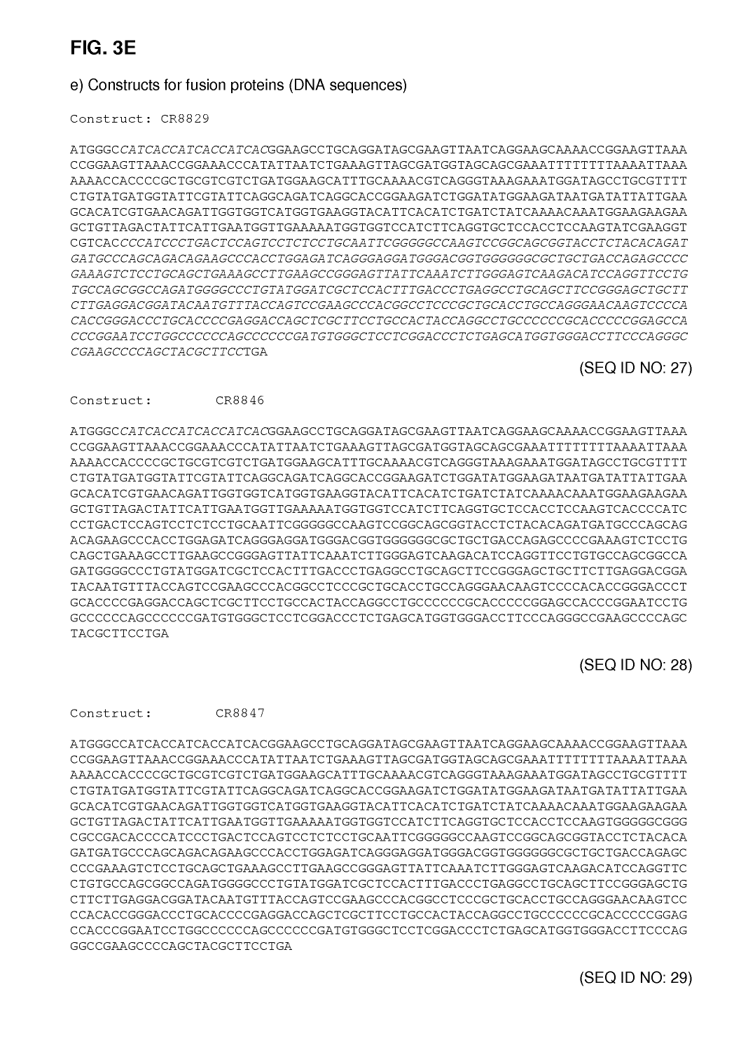

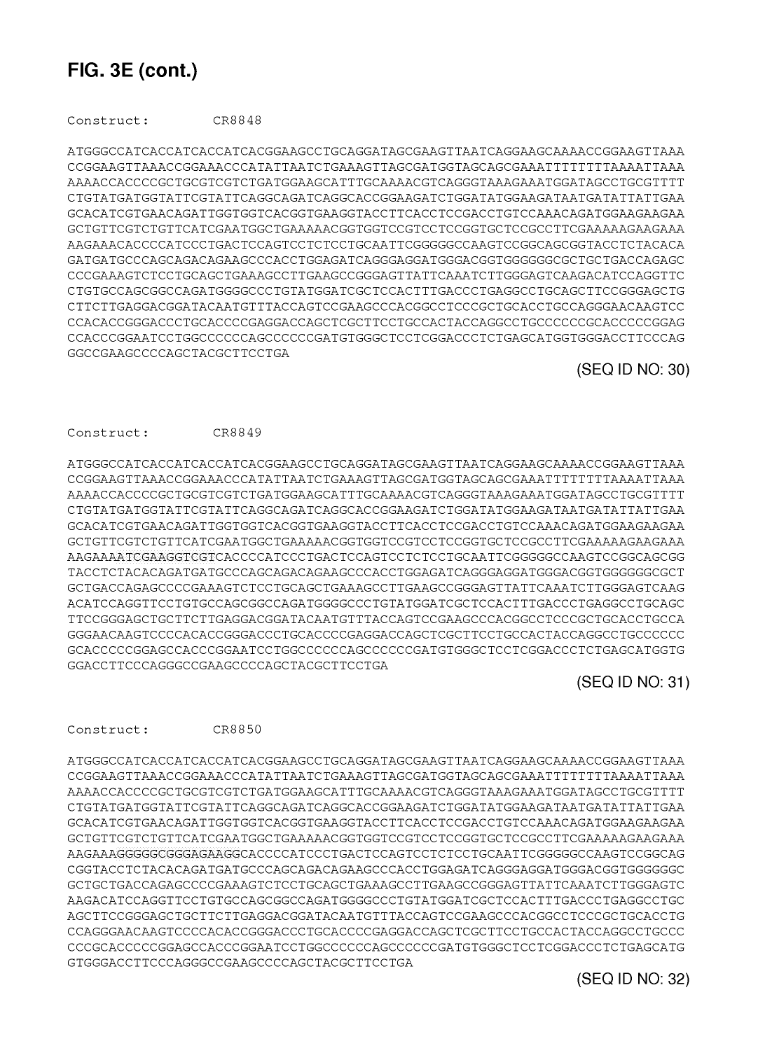

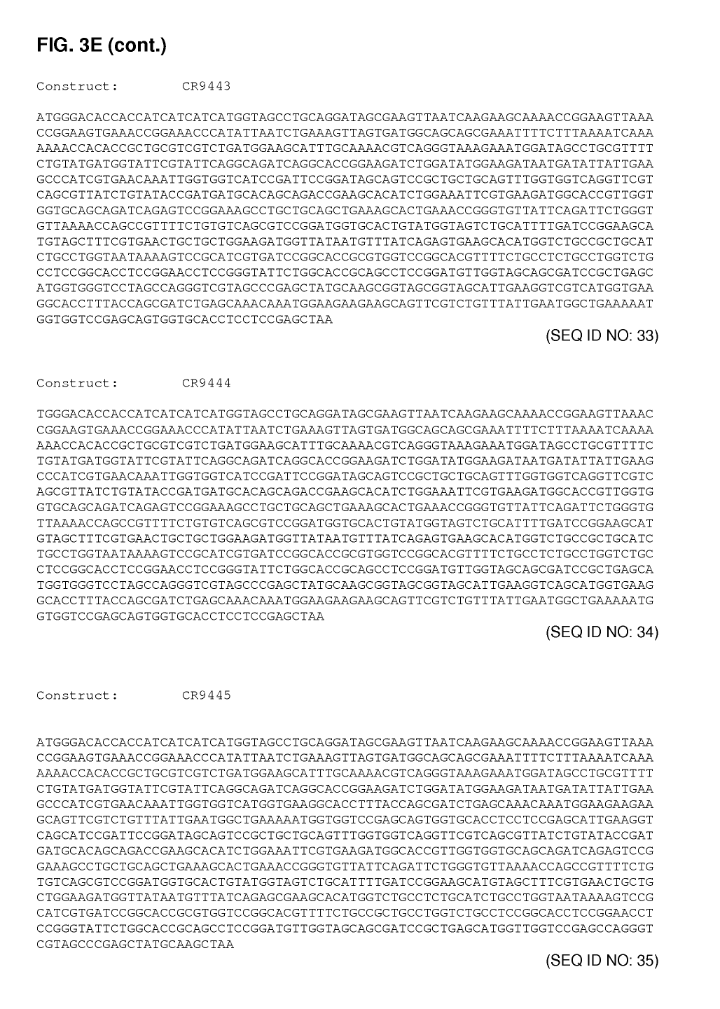

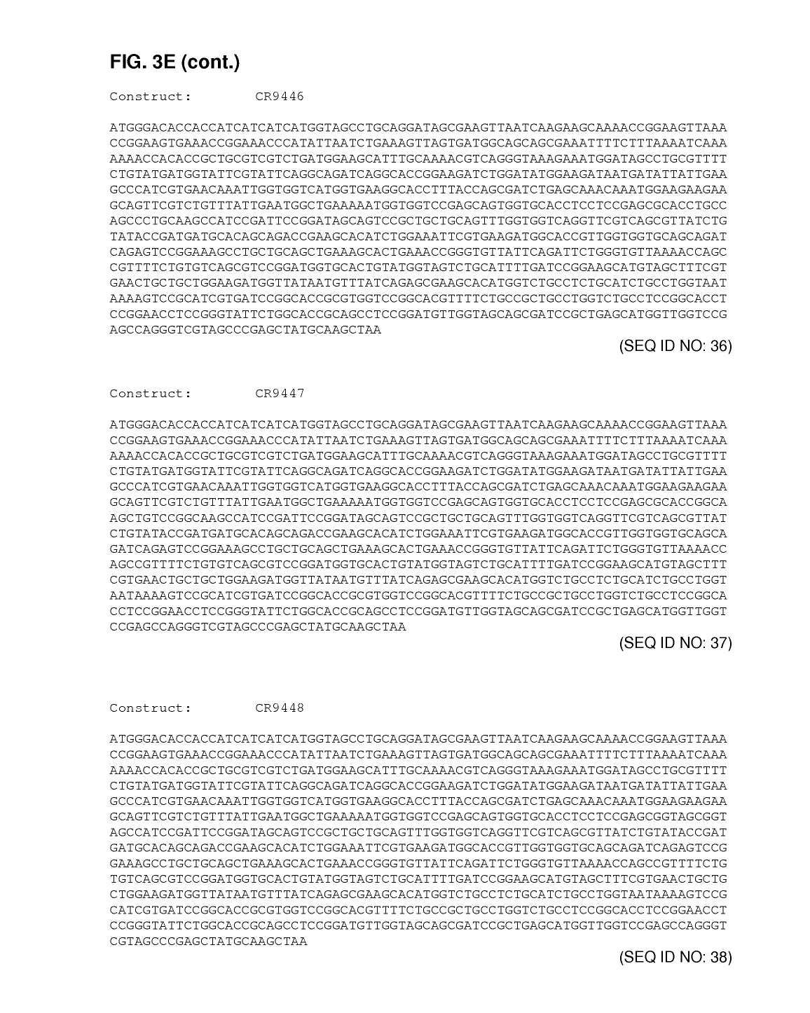

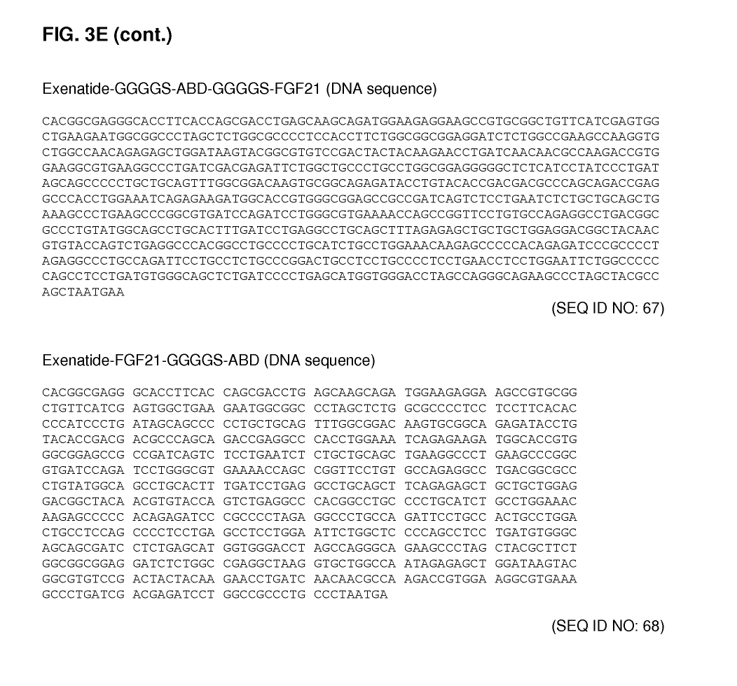

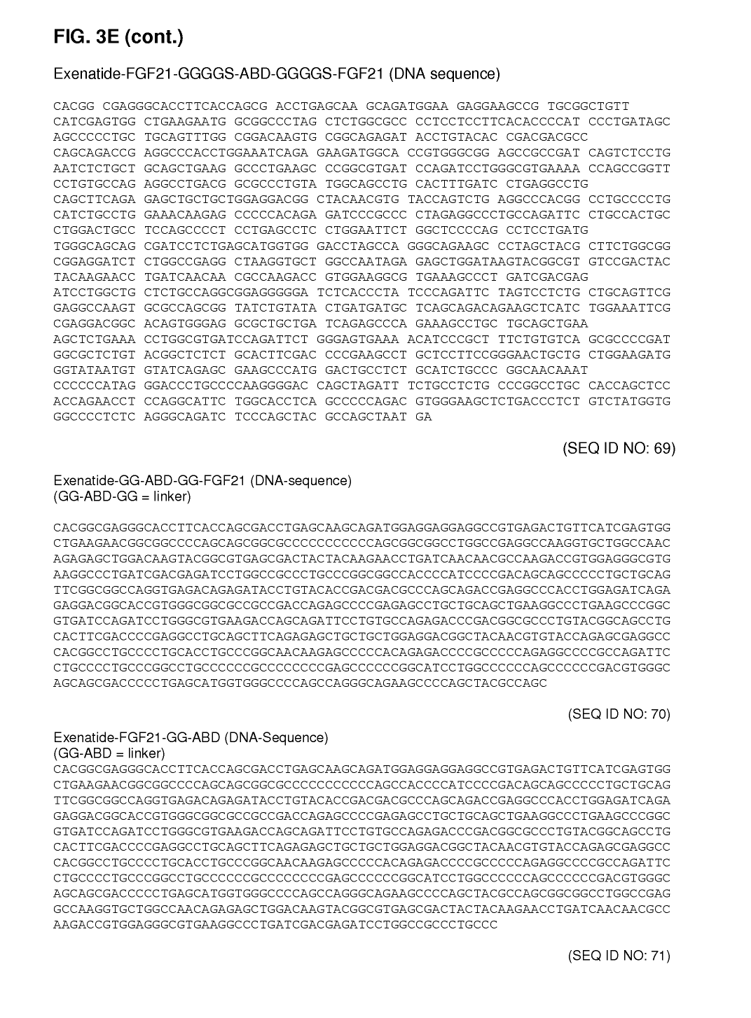

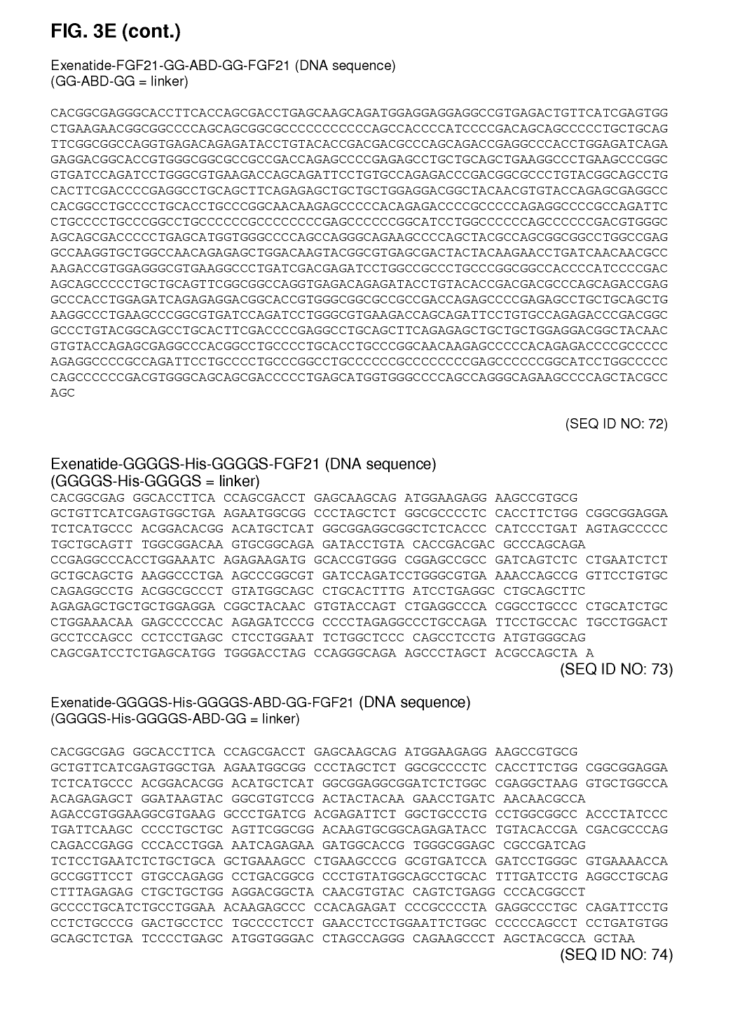

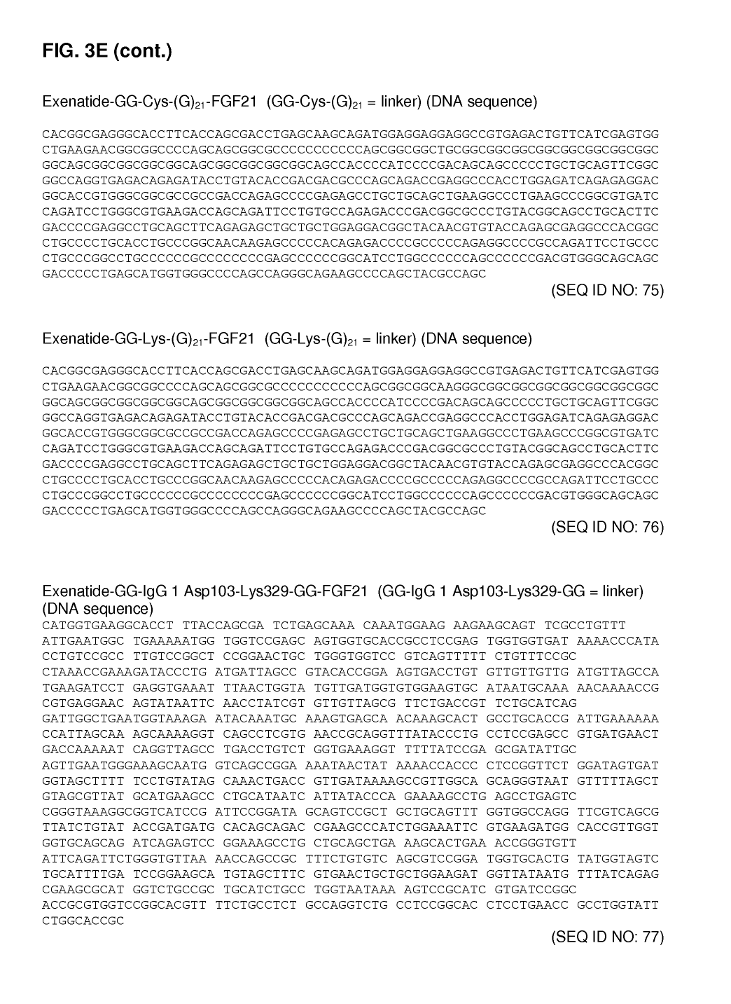

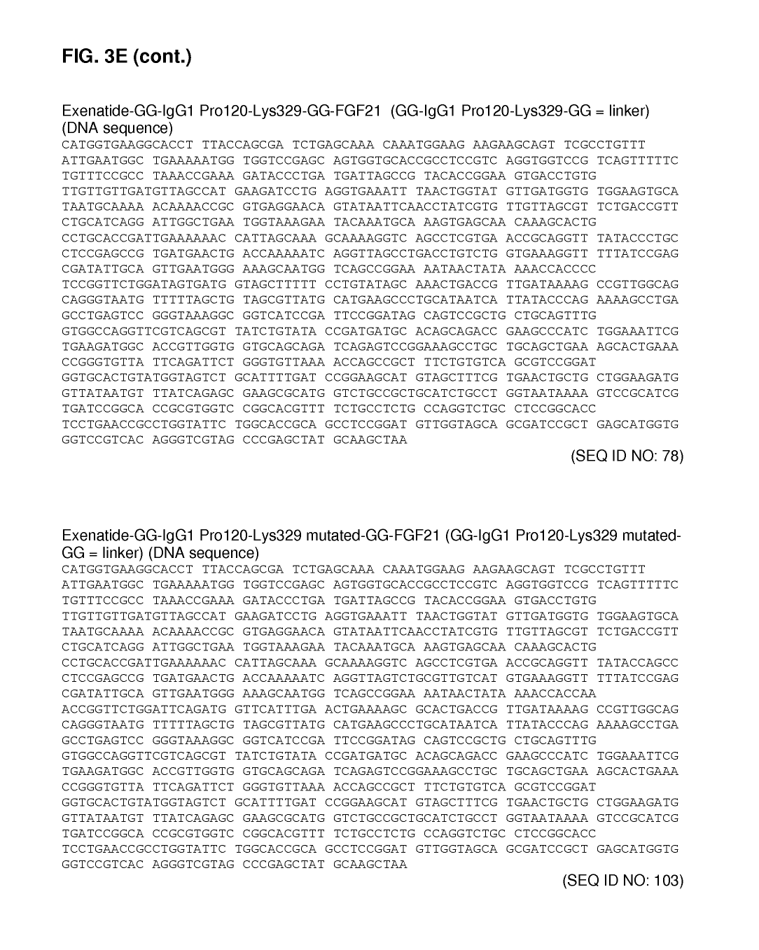

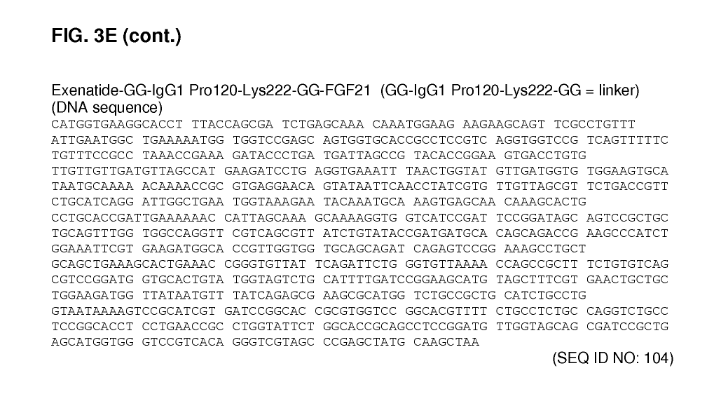

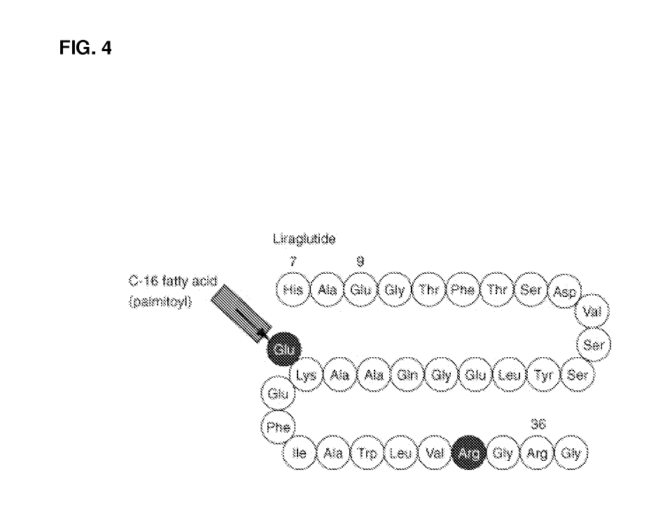

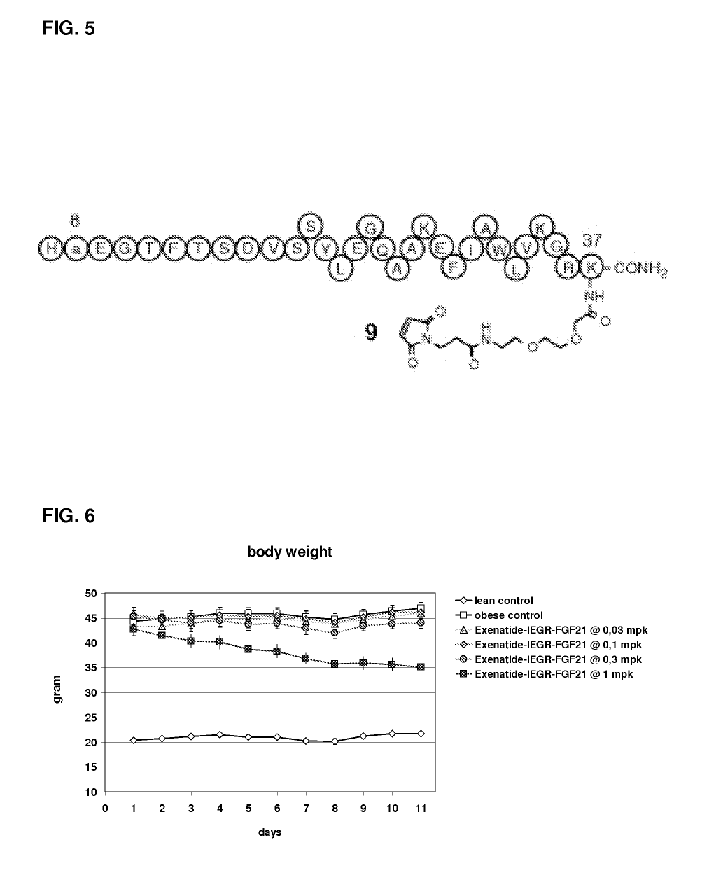

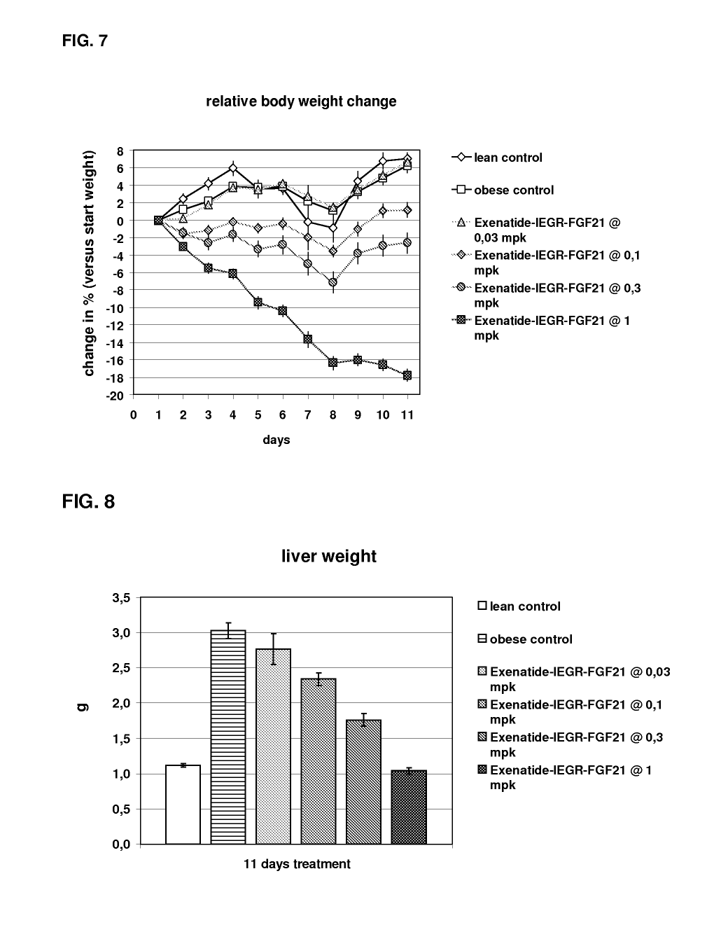

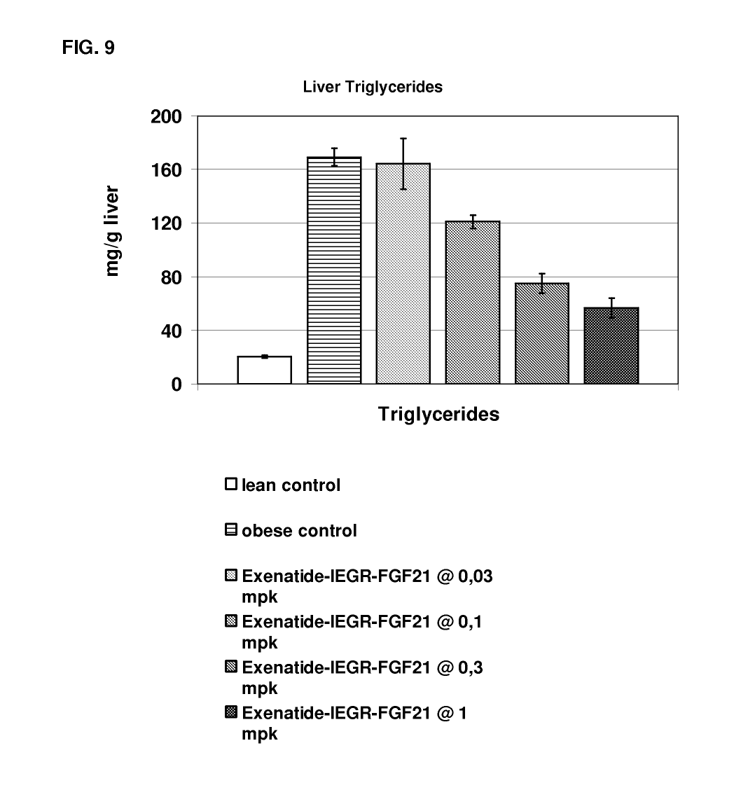

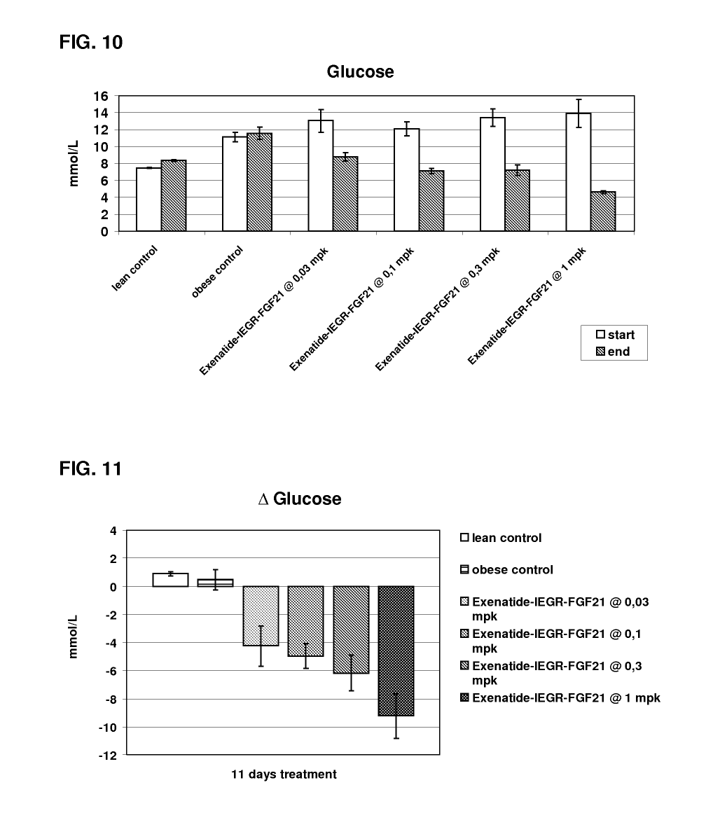

The cAMP content of cells was determined using a kit from Cisbio Corp. (cat. no. 62AM4PEC) based on HTRF (Homogenous Time Resolved Fluorescence). For preparation, cells were split into T175 culture flasks and grown overnight to near confluence in medium (DMEM/10% FBS). Medium was then removed and cells washed with PBS lacking calcium and magnesium, followed by proteinase treatment with accutase (Sigma-Aldrich cat. no. A6964). Detached cells were washed and resuspended in assay buffer (1× HBSS; 20 mM HEPES, 0.1% BSA, 2 mM IBMX) and cellular density determined. They were then diluted to 4×10⁵ cells/mL and 25 μL-aliquots dispensed into the wells of 96-well plates. For measurement, 25 μL of test compound in assay buffer was added to the wells, followed by incubation for 30 minutes at room temperature. After addition of HTRF reagents diluted in lysis buffer (kit components), the plates were incubated for 1 h, followed by measurement of the fluorescence ratio at 665/620 nm. In vitro potency of agonists was quantified by determining the concentrations that caused 50% activation of maximal response (EC₅₀). Results are summarized in table 1 and dose-response curves are shown in FIG. 1A.

3. In Vitro Cellular Assay for Human FGF-21 Receptor Efficacy and Activation of Downstream Signalling (In-Cell Western)

The cellular efficacy of FGF-21 or FGF-21 fusion proteins was measured using a specific and highly sensitive In-Cell Western (ICW) assay. The ICW assay is an immunocytochemical assay usually performed in microplate format.

CHO Flp-In cells (Invitrogen, Darmstadt, Germany) stable expressing the human FGFR1c together with human beta-Klotho (KLB) were used for FGF-21 receptor autophosphorylation assay using In-Cell Western [1]. In order to determine the receptor autophosphorylation level, 2×10⁴ cells/well were seeded into 96-well plates and grown for 48 h. Cells were serum starved with serum-free medium Ham's F-12 Nutrient Mix with GlutaMAX (Gibco, Darmstadt, Germany) for 3-4 h. The cells were subsequently treated with increasing concentrations of either human FGF-21, the indicated FGF-21 fusion protein, or other peptides for 5 min at 37° C. After incubation the medium was discarded and the cells were fixed in 3.7% freshly prepared para-formaldehyde for 20 min. Cells were permeabilized with 0.1% Triton-X-100 in PBS for 20 min. Blocking was performed with Odyssey blocking buffer (LICOR, Bad Homburg, Germany) for 2 h at room temperature. Anti-pFGFR Tyr653/654 (New England Biolabs, Frankfurt, Germany) was incubated overnight at 4° C. After incubation of the primary antibody, cells were washed with PBS+0.1% Tween20. The secondary anti-Mouse 800CW antibody (LICOR, Bad Homburg, Germany) was incubated for 1 h at room temperature. Subsequently cells were washed again with PBS+0.1% Tween20 and infrared dye signals were quantified with an Odyssey imager (LICOR, Bad Homburg, Germany). Results were normalized by quantification of DNA with TO-PRO3 dye (Invitrogen, Karlsruhe, Germany). Data were obtained as arbitrary units (AU) and EC₅₀ values were obtained from dose-response curves and are summarized in table 1. FIG. 1B shows the results from an ICW with CHO cells overexpressing human FGFR1c plus KLB.

To assess the activation of a downstream effector of FGFR signalling by FGF-21-GLP-1RA fusion proteins the phosphorylation of MAP kinases ERK1/2 were analysed. The same ICW protocol as described above was used, simply the primary antibody was replaced by anti-phospho-p44/42 MAPK (Erk1/2) (Thr202/Tyr204) (New England Biolabs, Frankfurt, Germany). FIG. 1C show the results from ICW with CHO cells overexpressing human FGFR1c plus KLB and detection of ERK1/2 phosphorylation. EC₅₀ values are summarized in table 1.

TABLE 1
In vitro EC₅₀ values of fusion proteins on human GLP-1R, human
FGFR1c plus KLB or the downstream effector MAP kinase ERK1/2.

| | hGLP-1R | pFGFR | pERK |
|---|---|---|---|
| | cAMP | ICW | ICW |
| | EC₅₀ | EC₅₀ | EC₅₀ |
| Compound | (pmol/L) | (nmol/L) | (nmol/L) |
| GLP-1(7-36) | 0.8 | n.d. | n.d. |
| Exenatide | 0.7 | n.d. | n.d. |
| Lixisenatide | 2.3 | n.d. | n.d. |
| FGF21 wild type | n.d. | 4.3 | 0.135 |
| Exenatide-FGF21 | 4.1 | 1.3 | 0.51 |
| Exenatide-IEGR-FGF21 | 4.0 | 1.9 | 0.40 |
| Exenatide-IEGQ-FGF21 | 6.1 | 35.4 | 0.79 |
| Exenatide-GSGS-FGF21 | 7.2 | 19.1 | 0.53 |
| Exenatide-GGGRR-FGF21 | 7.7 | 7.4 | 0.98 |
| Exenatide-APSPAS-FGF21 | 3.0 | 4.1 | 0.27 |
| Exenatide-APSCPAS-FGF21 | 13.2 | 193.3 | 10.9 |
| Exenatide-FGF21-GG-ABD | 7.96 | 79.8 | 89.9 |
| Exenatide-FGF21-GG-ABD- | 21.6 | 37.3 | 4.34 |
| GG-FGF21 | | | |
| Exenatide-GG-ABD-GG-FGF21 | 15.9 | n.d. | n.d. |
| Exenatide-GGGGS-His-GGGGS- | 2.54 | n.d. | 4.97 |
| ABD-GG-FGF21 | | | |
| Lixisenatide-FGF21 | 3.7 | 3.7 | 0.24 |
| Lixisenatide-IEGR-FGF21 | 3.8 | 3.1 | 1.00 |
| Lixisenatide-GGR-FGF21 | 3.6 | 2.6 | n.d. |
| FGF21-GSGSIEGR-Exenatide | 2,700 | 62.3 | 1.73 |
| FGF21-GSGSIEGQ-Exenatide | >10,000 | 33.0 | 1.67 |

4. Treatment of Ob/Ob Mice

Female ob/ob mice (B6.V-LEP OB/J, age of 10 weeks) were obtained from Charles Rivers Laboratories (Sulzfeld, Germany). Mice were randomly assigned to treatment or vehicle groups, and the randomization was stratified by body weight and fed blood glucose levels. The animals were housed in groups of 6 at 23° C. and on a 12 h light-dark cycle. All experimental procedures were conducted according to German Animal Protection Law. Mice were fed ad libitum with standard rodent chow during the drug treatment periods. Body weight and food intake was recorded every other day throughout the study.

Ob/ob mice were treated with vehicle (PBS), 0.15 mg·kg⁻¹·day⁻¹ exenatide (SEQ ID NO: 4), 0.75 mg·kg⁻¹·day⁻¹ recombinant human FGF-21 (SEQ ID NO: 2) or a combined dose of FGF-21 and exenatide (0.75+0.15 mg·kg⁻¹·day⁻¹), 0.9 mg·kg⁻¹·day⁻¹ Exenatide-IEGR-FGF-21 (SEQ ID NO: 3), or 0.9 mg·kg⁻¹·day⁻¹ Exenatide-FGF-21 (SEQ ID NO: 4) subcutaneously once daily. One day before the first treatment and at study day 10 blood glucose was measured by tail tip bleeding under fed conditions. As shown in FIG. 2A the blood glucose levels of the treated mice became normoglycaemic. On study day 8 a glucose tolerance test (OGTT) was performed. Fasted mice were orally challenged with 2 g·kg⁻¹ glucose. Blood glucose was measured at indicated time points by tail tip bleeding without anaesthesia. The results of the OGTT are shown in FIG. 2B. The calculated area under each curve (AUC) are shown in FIG. 2C. Compared to the administration of only FGF-21 or only exenatide glucose tolerance was markedly stronger improved by combination treatment and also normalized using two functional molecules in terms of a fusion protein.

5. Treatment of Ob/Ob Mice by Chronic Infusion

Female ob/ob mice (B6.V-LEP OB/J, age of 9 weeks) were obtained from Charles Rivers Laboratories (Sulzfeld, Germany). Mice were randomly assigned to treatment or vehicle groups, and the randomization was stratified by body weight and fed blood glucose levels. The animals were housed in groups of 8 at 23° C. and on a 12 h light-dark cycle. All experimental procedures were conducted according to German Animal Protection Law. Mice were fed ad libitum with standard rodent chow during the drug treatment periods. Body weight and food intake was recorded every other day throughout the study.

Ob/ob mice were treated with vehicle (PBS), 0.03, 0.1, 0.3, and 1.0 mg·kg⁻¹·day⁻¹ recombinant Exenatide-IEGR-FGF-21 (SEQ ID NO: 15) via chronic infusion by Alzet pumps (type 1004) over 11 days.

Treatment of ob/ob mice with the fusion protein Exenatide-IEGR-FGF-21 showed a dose dependent decrease of body weight with highest reduction of 17.8% at 1 mg/kg (FIGS. 6 and 7, table 2).

TABLE 2
Relative body weight change (%) of ob/ob mice after 11 days of treatment
Relative body weight change (%)

| | | |
|---|---|---|
| 0.03 | mg/kg | +6.6% |
| 0.1 | mg/kg | +1.1% |
| 0.3 | mg/kg | −2.6% |
| 1 | mg/kg | −17.8% |

At the end of the study liver weight and liver triglycerides were analysed. Total liver weight and liver triglycerides were dose-dependently decreased by treatment of ob/ob mice with the fusion protein (FIGS. 8 and 9).

Two days before pump implantation and after 11 days of treatment blood glucose was measured by tail tip bleeding under fed conditions. As shown in FIGS. 10 and 11 blood glucose levels of the chronic infused mice were decreased dose-dependently with highest effect at the dosage of 1.0 mg·kg⁻¹·day⁻¹ recombinant fusion protein. Even the lowest dose of 0.03 mg·kg⁻¹·day⁻¹ recombinant fusion protein resulted in normalization of blood glucose levels comparable to those of healthy lean control animals.

1. A fusion protein comprising the polypeptide with structure A-B-C or C-B-A or B-A-C or B-C-A or A-C-B or C-A-B or A-B-C-B-C or A-C-B or A-B-C-B or A-C-B-C, wherein A is a GLP-1R (glucagon-like peptide-1 receptor) agonist and C is an FGF-21 (fibroblast growth factor 21) compound and B is a linker comprising about 0 to 1000 amino acids.

2. The fusion protein according to claim 1, wherein the linker comprises a functional moiety conferring one or more additional functions beyond that of linking A and C.

3. (canceled)

4. The fusion protein according to claim 2, wherein the FGF-21 compound is selected from the group of native FGF-21, FGF-21 mimetic and SEQ ID NO: 3.

5. The fusion protein according to claim 4, wherein the FGF-21 mimetic is selected from a protein having at least about 80% amino acid sequence identity to the amino acid sequence shown in SEQ ID NO: 3 and having FGF-21 activity, a FGF-21 fusion protein and/or a FGF-21 conjugate.

6. (canceled)

7. (canceled)

8. The fusion protein according to claim 5, wherein the FGF-21 mimetic is selected from a FGF-21 mutein, a FGF-21-Fc fusion protein, a FGF-21-HSA fusion protein and/or a PEGylated FGF-21.

9. The fusion protein according to claim 8, wherein the GLP-1R agonist is selected from a bioactive GLP-1, a GLP-1 analogue, a GLP-1 substitute, GLP-1(7-37), GLP-1(7-36)amide, exendin-4, liraglutide, CJC-1131, albugon, albiglutide, exenatide, exenatide-LAR, oxyntomodulin, lixisenatide, geniproside, or a short peptide with GLP-1R agonistic activity.

10. (canceled)

11. The fusion protein according to claim 9, wherein the linker comprises one or more of the following functional moieties a) to h):
a) a moiety conferring increased stability and/or half-life to the fusion such as an XTENylation or PASylation sequence or Elastin-like polypeptides (ELPs);
b) an entry site for covalent modification of the fusion protein such as a cysteine or lysine residue;
c) a moiety with intra- or extracellular targeting function such as a protein-binding scaffold;
d) a protease cleavage site such as a Factor Xa cleavage site or a cleavage site for another extracellular protease;
e) a Fc portion of an immunoglobulin, e.g. the Fc portion of IgG4;
f) HSA;
g) an amino acid sequence comprising one or more histidine; and
h) an albumin binding domain (ABD).

12. (canceled)

13. (canceled)

14. The fusion protein according to claim 11, wherein the linker comprises one or more of the following protease cleavage sites:
a) a factor Xa cleavage site and preferably comprising or consisting of the sequence IEGR (SEQ ID NO:11); and
b) a protease cleavage site and preferably comprising or consisting of at least one arginine and more preferably comprising or consisting of the sequence GGGRR (SEQ ID NO: 14).

15. The fusion protein according to claim 14, wherein the linker comprises or consists of an entry site for covalent modification and preferably comprising or consisting of the sequence according to SEQ ID NO:13.

16. The fusion protein according to claim 15, wherein the linker comprises or consists of a protein stabilisation sequence and preferably comprises a PASylation sequence selected from the group of: SEQ ID NO: 12, SEQ ID NO: 95, SEQ ID NO: 96, SEQ ID NO: 97, SEQ ID NO: 98, SEQ ID NO: 99, SEQ ID NO: 100, and SEQ ID NO: 101.

17. (canceled)

18. (canceled)

19. The fusion protein according to claim 16, comprising one or more moieties D being covalently attached to the entry site(s) for covalent modification of the linker, wherein the covalently attached moiety or moieties D are selected from the list consisting of:
a) a targeting unit such as an antibody or protein-binding scaffold;
b) a protein-stabilizing unit such as a hydroxyethyl starch derivative (HES) or a polyethylenglycol or derivative thereof (PEG or PEG derivative) and
c) a fatty acid.

20. The fusion protein according to claim 19, comprising a tag for protein-purification such as a His-tag and wherein the tag is preferably N- or C-terminally attached to the fusion protein.

21. The fusion protein according to claim 20 comprising a protease cleavage site between the protein-purification tag and the remaining parts of the fusion protein, wherein the protease cleavage site is preferably a Sumo protease cleavage site.

22. The fusion protein according to claim 21, wherein A is an FGF-21 mutein and C is exenatide, exendin-4 or lixisenatide.

23. The fusion protein according to claim 22, wherein B has a sequence selected from the group of: SEQ ID NO: 11, SEQ ID NO: 12, SEQ ID NO: 13, SEQ ID NO: 14, SEQ ID NO: 95, SEQ ID NO: 96, SEQ ID NO: 97, SEQ ID NO: 98, SEQ ID NO: 99, SEQ ID NO: 100, and SEQ ID NO: 101.

24. The fusion protein according to claim 23, wherein A is an FGF-21 mutein comprising or consisting of SEQ ID NO: 102.

25. The fusion protein according to claim 24, wherein C is exenatide.

26. (canceled)

27. A pharmaceutical composition comprising the fusion protein of claim 1 together with a pharmaceutically acceptable excipient.

28. A pharmaceutical composition comprising the fusion protein of claim 1 together with a pharmaceutically acceptable excipient for use as a medicament.

29. Article of manufacture comprising
a) a pharmaceutical composition according to claim 27 and
b) a container or packaging material.

30. A method of treating a disease or disorder of a patient, in which the increase of FGF-21 receptor autophosphorylation or in which the increase of FGF-21 efficacy is beneficial for the curing, prevention or amelioration of the disease or disorder, wherein the method comprises administration to the patient of a fusion protein of claim 1.

31. A method of treating a cardiovascular disease and/or diabetes mellitus and/or at least one metabolic syndrome which increases the risk of developing a cardiovascular disease and/or diabetes mellitus, preferably Type 2-diabetes in a patient comprising the administration to the patient of a fusion protein of claim 1.

32. A method of lowering plasma glucose levels, of lowering the lipid content in the liver, of treating hyperlipidemia, of treating hyperglycemia, of increasing the glucose tolerance, of decreasing insulin tolerance, of increasing the body temperature, and/or of reducing weight of a patient comprising the administration of a fusion protein of claim 1.

33. A nucleic acid encoding the fusion protein according to claim 1, optionally comprising one of the following nucleic acid sequences:
a) a nucleic acid sequence according to one of the sequences with ID NOs: 27 to 38;
b) a nucleic acid coding for a protein sequence according to SEQ ID NOs: 15 to 26 and 39 to 44; and
c) a nucleic acid hybridizing under stringent conditions with a nucleic acid according to a) or b).

34. A vector comprising the nucleic acid of claim 33 suitable for expression of the encoded protein in a eukaryotic or prokaryotic host.

35. A cell stably or transiently carrying the vector of claim 1 and capable of expressing the fusion protein under appropriate culture conditions.

36. A method of preparing the fusion protein of claim 1 comprising
a) cultivating a culture of cells under appropriate culture conditions for the fusion protein to be expressed in the cell, or
b) harvesting or purifying the fusion protein from a culture comprising cells that have been cultivated under appropriate conditions for the fusion protein to be expressed, or
c) cultivating the cells according to step a) and purifying the fusion protein according to step b); and optionally
d) cleaving of a His-tag using a protease of fusion protein.
